(19) United States
(12) Patent Application Publication
(10) Pub. No.: US 2003/0032078 A1
(43) Pub. Date: Feb. 13, 2003

(54) Methods and compositions for the treatment of macular and retinal degenerations

(75) Inventor: Gabriel H. Travis, Los Angeles, CA (US)

Correspondence Address: Gina N. Shishima, Fulbright & Jaworski L.L.P., Suite 2400, 600 Congress Avenue, Austin, TX, 78701, US

(73) Assignee: Board of Regents, The University of Texas System

(21) Appl. No.: 09/885,303

(22) Filed: Jun. 19, 2001

Related U.S. Application Data
(60) Provisional application No. 60/263,837, filed on Jan. 23, 2001.

(51) Int. Cl. C12Q 1/32; C12N 9/04

(52) U.S. Cl. 435/26; 435/190

(57) ABSTRACT

The present invention is a method for screening and identifying therapeutic agents for the treatment of macular or retinal degeneration. The candidate substances preferably reduces the activity of 11-cis-retinol dehydrogenase. In vitro and in vivo studies administering the inhibitor molecules to abcr knockout mice and analyzing for the inhibition of lipofuscin (A2E) accumulation are contemplated.

The present application claims the benefit of U.S. Provisional Application Serial No. 60/263,837 filed on Jan. 23, 2001. The entire text of the above-referenced disclosure is herein incorporated by reference. The government may own rights in the present invention pursuant to grant number R01 EY11713 from The National Institutes of Health.

### BACKGROUND OF THE INVENTION

1. Field of the Invention The present invention relates generally to the field of optical disease, such as macular and retinal degenerations. More particularly, it concerns modulators of dehydrogenases, such as 11-cis-retinol dehydrogenase, which are involved in the visual pathway and can be used for the prevention or treatment of macular or retinal degeneration.

2. Description of Related Art

The macular degenerations are a group of inherited blinding diseases that cause destruction of the central retina. Age-related macular degeneration (AMD), for example, is the leading cause of blindness in the elderly and affects ten million people in the United States. Little is understood about the cause of macular degeneration, and there exists no effective treatment. The macula contains a central structure called the fovea, populated exclusively with cone photoreceptors. Death of these foveal cones results in devastating loss of central vision. Although cones are far more important than rods for human vision, most of what is known about photoreceptor function comes from the study of rod-dominant retinas. For example, the majority of genes identified for inherited retinal degenerations encode proteins expressed specifically in rods. The current invention focuses on cones and diseases of the macula. Fifteen distinct loci for inherited diseases involving abnormal cone function and macular degenerations have been mapped but not yet cloned.

Age-related macular degeneration (AMD) is related to Stargardt's disease (STGD), but it is a more prevalent disorder causing visual loss in the elderly. The estimated frequency of AMD is 2% at 70 and 6% at 80 years (Hawkins et al., 1999). Similar to STGD, AMD is associated with central visual loss, delayed dark adaptation, lipofuscin accumulation in the RPE, and degeneration of photoreceptors (Dorey et al., 1989; Steinmetz et al., 1993; Kliffen et al., 1997; Midena et al., 1997; Delori et al., 2000). By ophthalmological exam, whitish spots called drusen are seen early in the disease. Although the etiology of AMD is complex, a strong genetic component has been demonstrated (Heiba et al., 1994; Meyers et al., 1994; Klein et al., 1994; Klaver et al., 1998). Recently, mutations in the ABCR gene were associated with AMD in a subset of cases (Allikmets et al., 1997; Simonelli et al., 2000; Allikmets et al., 2000), although the frequency of this association has been debated (Stone et al., 1998; De La Paz et al., 1999; Souied et al., 2000). Thus, homozygous mutations in the ABCR gene cause a severe blinding disease of childhood while heterozygous mutations cause gradually developing blindness in the elderly.

Stargardt's disease (STGD) is a recessive form of macular degeneration with an onset during childhood and an estimated prevalence of 1:10,000 (Blacharski et al., 1988). STGD is characterized by progressive loss of central vision leading to blindness (Stargardt et al., 1909). Early in the disease course, patients show delayed dark adaptation but otherwise normal function of rod photoreceptors (Fishman et al., 1991). Histologically, STGD is associated with deposition of lipofuscin in cells of the retinal pigment epithelium (RPE) (Bimbach et al., 1994; De Laey et al., 1995). Degeneration of the RPE occurs subsequently, with photoreceptor death appearing late in the disease. STGD is caused by mutations in the newly identified ABCR gene (Allikmets et al., 1997; Stone et al., 1998; Lewis et al., 1999).

There is a continued need for the development of treatment and preventative options for macular and retinal degeneration. Current treatment regimens for macular degeneration vary from dietary supplementation with zinc or lutein to different forms of surgery. The successfulness of these treatments is debatable, rendering an ongoing pursuit for effective alternatives for the prevention and treatment of macular and retinal degeneration. Early detection of macular degeneration is also becoming increasingly important. Photodynamic therapy, a surgical treatment for some cases of macular degeneration, is only beneficial before extensive vision loss has occurred (Bressler, et al., 2000).

### SUMMARY OF THE INVENTION

The present invention is a method for screening and identifying therapeutic agents to treat or preventblindness in humans due to macular or retinal degeneration, including Stargardt's macular degeneration. The strategy is based on the previous observations: (i) forms of macular and retinal degenerations, including those caused by mutations in the ABCR gene, result from the accumulation of toxic lipofuscin in cells of the retinal pigment epithelium; (ii) A2E, the major fluorophore of lipofuscin, is formed in these diseases due to excess production of the visual-cycle retinoid, all-trans-retinaldehyde, a precursor of A2E; and (iii) A2E formation can be strongly suppressed by raising mice in darkness, which prevents the formation of all-trans-retinaldehyde. The strategy for screening and identifying therapeutic agents may involve one or more of the the following steps: (i) Screen candidate molecules for inhibition of dehydrogenase (i.e. 11-cis-retinol dehydrogenase) activity in an in vitro assay; (ii) administer effective inhibitor molecules from step (i) to abcr31 /− knockout mice and analyze for effect, such as the inhibition of lipofuscin (A2E) accumulation in vivo in the retinal pigment epithelium; and (iii) begin clinical trials on active molecules (from (ii)) in patients with Stargardt's and other macular degenerations, after confirming an acceptable profile of side effects.

An embodiment of the invention comprises contacting a short chain dehydrogenase with a candidate substance; and determining whether the candidate substance reduces the activity of the dehydrogenase. Another embodiment comprises contacting a dehydrogenase capable of oxidizing 11 cis-retinol to 11-cis-retinaldehyde with a candidate substance; and determining whether the candidate substance reduces the activity of the dehydrogenase.

This determination is preferably done by measuring the rate of dehydrogenated product formation or by evaluating the amount of transfer of a tritiated label from nicotinamide adenine dinucleotide (NAD+) to the hydrogenated NADH. The dehydrogenase is preferably a short chain dehydrogenase such as a 11-cis-retinol dehydrogenase and is preferably enzymatically active. The dehydrogenases of the current invention are able to recognize 11-cis-retinol and oxidize 11-cis-retinol to 11-cis-retinaldehyde. It is an aspect of the invention that the 11-cis-retinol dehydrogenase comprises at least 30 contiguous amino acids having an amino acid sequence of SEQ ID NO: 4 or is encoded from a region of at least 30 bases of the nucleic acid of SEQ ID NO: 3. It is preferable that the candidate substance is contacted with the dehydrogenase in vitro or in vivo. The candidate substance may be a small molecule such as 13-cis-retinoic acid, peptide, polypeptide such as an antibody, or nucleic acid molecule. In some embodiments of the invention, the candidate substance comprises a ribozyme or antisense molecule which may comprise a portion of the coding sequence of the dehydrogenase.

An aspect of the invention includes the optional steps comprising contacting a first animal lacking a functional abcr gene with the candidate substance; and determining the amount of a component of lipofuscin such as A2-E in a retinal pigment epithelium cell. Another optional step comprises comparing the amount of lipofuscin from the first animal with the amount of lipofuscin in a retinal pigment epithelium cell from a second animal lacking a functional abcr gene in the absence of the candidate substance. The animal may be a mouse or a mouse with a knockout mutation in an abcr gene. It is an aspect of the invention that the dehydrogenase is produced recombinantly.

Another embodiment of the invention comprises a method of screening for a therapeutic agent for the treatment of macular or retinal degeneration comprising contacting a first cell which is preferably a recombinant cell expressing 11-cis-retinol dehydrogenase with a candidate substance and determining whether the activity or amount of 11-cis-retinol dehydrogenase is reduced in the first cell compared to a second cell which is preferably a recombinant cell expressing 11-cis-retinol dehydrogenase but not contacted with the candidate substance. The amount of 11-cis-retinol dehydrogenase can determined by a variety of methods including measuring 11-cis-retinol dehydrogenase protein levels or measuring 11-cis-retinol dehydrogenase transcript levels, The activity of 11-cis-retinol dehydrogenase can be described as enzymatic activity. A further aspect of this method comprises transfecting the first and second cell, or a parent thereof, with recombinant 11-cis-retinol dehydrogenase prior to contacting the first cell with a candidate substance.

Yet another embodiment of the invention comprises a method of screening for a therapeutic agent for the treatment of macular or retinal degeneration comprising the following steps, wherein steps (a)-(d), (a)-(f), or (a)-(g) are performed, where the steps comprise:

(a) contacting a first rod cell with a candidate substance;

(b) determining the amount of lipofuscin or a component of lipofuscin in the first rod cell;

(c) determining the amount of lipofuscin or a component of lipofuscin in a second rod cell not exposed to the candidate substance;

(d) comparing the amount of lipofuscin or a component of lipofuscin between the first rod cell and the second rod cell;

(e) contacting a first animal lacking a functional abcr gene with the candidate substance;

(f) determining the amount of lipofuscin or a component of lipofuscin in a retinal pigment epithelium cell of the first animal; and

(g) comparing the amount of lipofuscin or a component of lipofuscin from the first animal with the amount of lipofuscin or a component of lipofuscin in a retinal pigment epithelium cell from a second animal lacking a functional abcr gene in the absence of the candidate substance.

Yet another embodiment of the current invention comprises a method of preparing a therapeutic agent for the treatment of macular or retinal degeneration in a subject comprising contacting 11-cis-retinol dehydrogenase with a candidate substance; determining whether the candidate substance reduces the activity of the dehydrogenase; and formulating the candidate substance in a pharmaceutically acceptable formulation, wherein the candidate substance reduces the activity of the dehydrogenase. Preferably, the 11-cis-retinol dehydrogenase comprises at least 30 contiguous amino acids having an amino acid sequence of SEQ ID NO: 4 or is encoded from a region of at least 30 bases of the nucleic acid of SEQ ID NO: 3. It is preferred that the candidate substance is formulated for opthalmic applications. The method also may comprise contacting a first animal lacking a functional abcr gene with the candidate substance; and determining the amount of a component of lipofuscin such as A2-E in a retinal pigment epithelium cell.

In further embodiments of the present invention, a subject with macular or retinal degeneration may be treated wherein the treatment comprises administering to the subject a therapeutically effective amount of an inhibitor of 11-cis-retinal dehydrogenase, wherein 11-cis-retinal dehydrogenase activity is reduced. The inhibitor may be a small molecule such as 13-cis-retinoic acid, peptide, polypeptide such as an antibody, or nucleic acid molecule. In some embodiments of the invention, the antibody comprises a ribozyme or antisense molecule which may comprise a portion of the coding sequence of the dehydrogenase. It is a further aspect of the invention that the subject has age-related macular degeneration, Stargardt's disease, cone-rod dystrophy, retinitis pigmentosa, or fundus flavimaculatus. The inhibitor may be directly administered to an eye afflicted with macular or retinal degeneration or it may be perfused into an eye afflicted with macular or retinal degeneration. The inhibitor may be administered to a subject once, twice, 3 times, 4 times, 5 times, 6 times, 7 times, 8 times, 9 times, 10 times, or more than 10 times. Administration may be repeated every 1 month, 3 months, 6 months, year, or after any other length of time wherein repeating the therapy is necessary. In some embodiments, surgery such as laser photocoagulation therapy or photodynamic therapy may be performed on the subject or an anti-angiogenic factor may be administered to the subject. An “effective amount” refers to an amount that achieves a desired goal, such as a reduction in 11-cis-retinal dehydrogenase activity. In some embodiments, an amount of a substance is administered to achieve a therapeutic effect (“therapeutically effective amount”), such as to reduce or eliminate the symptoms of macular or retinal degeneration, which are well documented.

An embodiment of the invention comprises a method of treating a subject with macular or retinal degeneration comprising administering to the subject a therapeutically effective amount of an 11-cis-retinal dehydrogenase.

It is contemplated that the methods of the current invention may be used for the prevention as well as the treatment of macular or retinal degeneration. Any method described herein may be modified for use in preventing or reducing the risk of acquiring macular or retinal degeneration.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A—Visual Cycle on Rod Dominant Retinas. Absorption of a photon (hv) by rhodopsin in a rod outer-segment induces 11-cis and all-trans isomerization of the retinal chromophore, forming active metarhodopsin II (MII*). After several seconds, MII* decays, releasing atRAL from the opsin apoprotein. Transport of atRAL from the disc interior is facilitated by the RmP flippase. Following photoisomerization and reduction by atRDH, the resulting atROL is released by the photoreceptor, diffuse the short distance across the extracellular space, and is taken up by the RPE cell where it is esterified by atRE by lectithin-retinol acyltransferase (LRAT). IMH captures the energy of ester hydrolysis to isomerize atROL into 11cROL. Finally, 11cROL is oxidized to 11cRAL by 11cRDH5 in RPE cells. 11cRAL diffuses across the extracellular space, is taken up by the outer segment, and recombines with opsin to regenerate rhodopsin.

FIG. 1B—Alternative visual cycle in cone photoreceptors. Absorption of a photon induces 11-cis to all-trans isomerization of the retinal chromophore, resulting in activated cone opsin (MII*). Decay of MII* releases atRAL, which is reduced to atROL by atRDH. atROL released into the extracellular space by cones (and rods) is taken up by Muller cells, which contain the novel atRI activity. This enzyme isomerizes atROL to 11cROL, possibly using fatty acyl CoA as an energy source. The 11cROL is subsequently esterified by a novel 11cRE-synthase. Hydrolysis of the stored 11cRE presumably involves retinyl ester hydrolase (REH) activity. 11cROL is released into the extracellular space, where it is taken up specifically by cone cells, which contain the novel 11cRDH activity. Rods lack 11cRDH activity and hence cannot utilize 11cROL. The resulting 11cRAL combines with opsin apoprotein to regenerate the visual pigment.

FIG. 2—HPLC analysis showing A2PE-H₂ in rod outer-segment (ROS) and RPE. (FIG. 2A) Chromatogram of phospholipid extracts from 12-week-old abcr+/+ and abcr−/− ROS. Detection wavelength is 500 mn. Inset shows absorption spectrum of A2PE-H₂ peak fraction, indicated by an arrow. (FIG. 2B) Histogram showing levels of A2PE-H₂ in ROS from abcr−/− mice at the indicated ages in area units per eye. Error bars show standard deviations. (FIG. 2C) Chromatogram of phospholipid extracts from 12-week-old abcr+/+ and abcr−/− RPE. Detection wavelength is 500 nm. Inset shows absorption spectrum of the A2PE-H₂ peak fraction, indicated by an arrow. (FIG. 2D) Histogram showing the relative levels of A2PE-H₂ in RPE from abcr−/− mice at the indicated ages in area units per eye±standard deviations. Note that A2PE-H₂ is exclusively present in ROS and RPE from abcr−/− mice.

FIG. 3—HPLC analysis showing the A2PE intermediate in the formation of A2-E. (FIG. 3A) Chromatogram of A2PE-H₂ purified from 11-month abcr31 /− outer segments. Detection wavelength is 500 nm. Inset shows spectrum of the A2PE-H₂ peak, labeled with an arrow. (FIG. 3B) Chromatogram of A2PE-H₂ fraction from FIG. 3A after 5 min incubation in HCl. Detection wavelength is 430 nm. The A2PE-H₂ peak is labeled with an arrow. Inset shows spectrum of the A2PE peak, labeled with the purple arrow. (FIG. 3C) Chromatogram of A2PE-H₂ fraction from FIG. 3A after overnight incubation in HCl. Detection wavelength is 430 nm. The phosphatidic acid peak is labeled with an arrow. Inset shows spectrum for the A2-E peak, labeled with an arrow. Note the disappearance of A2PE-H₂ and A2PE, and the appearance of phosphatidic acid and A2-E. (FIG. 3D) Mass spectrum of A2-E fraction from FIG. 3C. Note the major molecular-ion species with a m/z ratio of 592.3. The additional labeled peaks were also present in a sample containing only solvent. (FIG. 3E) Chromatogram of phospholipid extract from six-month-old abcr−/− RPE. Detection wavelength is 430 nm. Inset shows spectra for the A2PE peak, labeled with the purple arrow, and A2PE-H₂ peak, labeled with an arrow.

FIG. 4—HPLC analysis of phospholipid extracts from human retina and RPE showing phosphatidylethanolamine, A2PE-H₂, and A2-E accumulation. (FIG. 4A) and (FIG. 4D) show analysis of retina and RPE, respectively, from a 71-year-old female with no retinal pathology as a representative control. (FIG. 4B) and (FIG. 4E) show analysis of retina and RPE, respectively, from a 62-year-old female with FFM. (FIG. 4C) and (FIG. 4F) show analysis of retina and RPE, respectively, from a 73-year-old male with STGD1. Chromatograms are shown at detection wavelengths of 500 and 205-nm to display retinoid-conjugate and phospholipid absorption, respectively. For FIG. 4A-F, the 205-nm scale on the left is in absorption units (AU) and the 500-nm scale on the right is in milliabsorption units (mAU). Peaks corresponding to phosphatidylethanolamine (PE) in the retina samples are labeled with the arrows. Insets show absorption spectra for the labeled A2PE-H₂ and A2-E peaks.

FIG. 5—A2-E in RPE from mice raised under different light conditions. Data expressed in pmoles A2-E per eye from mice of the indicated ages. (FIG. 5A) Wild-type (abcr+/+) mice raised under cyclic light (open circles) or in total darkness (filled circles). (FIG. 5B) abcr−/− mutant mice raised under cyclic light (open circles) or in total darkness (filled circles). (FIG. 5C) abcr−/− mutant mice raised for 12-weeks under cyclic light and transferred to total darkness until reaching the indicated ages. Error bars show standard deviations. An asterisk (*) indicates a significant difference between data points by Student's t-test (p <0.05).

FIG. 6—Hypothetical biosynthetic pathway for A2-E in mice and humans lacking the RmP transporter. This model is based on the proposed scheme for A2-E biogenesis by Parish et al. (1998). In outer segments following a photobleach, phosphatidylethanolamine and all-trans-RAL are transiently in equilibrium with APE (reaction 1). After a [1,5] sigmatropic rearrangement, the resulting secondary amine may condense with another molecule of all-trans-RAL. Subsequent [3,3] sigmatropic rearrangement of the bis-retinoid product results in the formation of A2PE-H₂ (reaction 2), an irreversible step at neutral pH. Oxidation of A2PE-H₂ to A2PE (reaction 3) occurs within RPE phagolysosomes, and is accompanied by a shift in the visible λₘₐₓ from 500 to 430 nm. This blue-shift is expected, since the two tetraene side-chains are oriented meta on the pyridinium ring, hence resonance delocalization occurs at a higher energy in the oxidized form due to loss of aromaticity. Finally, A2-E is formed within RPE phagolysosomes upon acid-hydrolysis of the phosphate ester and release of phosphatidic acid (reaction 4).

FIG. 7—A2E and A2PE-H₂ in abcr−/− ocular tissues following treatment with isotretinoin. A2E and A2PE-H₂ were extracted from the indicated tissues and analyzed by HPLC as previously described (Mata et al., 2000; Weng et al., 1999). (FIG. 7A) A2E levels in abcr−/− RPE from four-month-old mice treated for one month with isotretinoin at 40 mg/kg/day (4-mo 13cRA), four-month-old mice treated with DMSO carrier alone (4-mo DMSO), and three-month-old untreated mice (3-mo untreated). (FIG. 7B) A2PE-H₂ levels in abcr−/− RPE from the same animals in panel (A) plus four-month-old untreated mice (4-mo untreated). (FIG. 7C) A2PE-H₂ levels in abcr−/− retinas from the same animals in panel (B). All data are shown as milliabsorption units (mAU) at the indicated wavelengths±SEM (n=3). Note approximately two-fold suppression of A2E accumulation in RPE, approximately three-fold suppression of A2PE-H₂ accumulation in RPE, and the near absence of A2PE-H₂ in retina following treatment with isotretinoin.

### DESCRIPTION OF ILLUSTRATIVE EMBODIMENTS

The present invention concerns the visual cycle (cycle for regenerating rhodopsin), particularly in rod cells, and provides methods and compositions for modulating components of that pathway. It takes advantage of the observations that dehydrogenases, such as the short chain dehydrogenase 11-cis-retinol dehydrogenase, are implicated in this cycle and that inhibitors of these enzymes are candidate agents for the treatment of macular and retinal degenerations.

I. The Visual Cycle

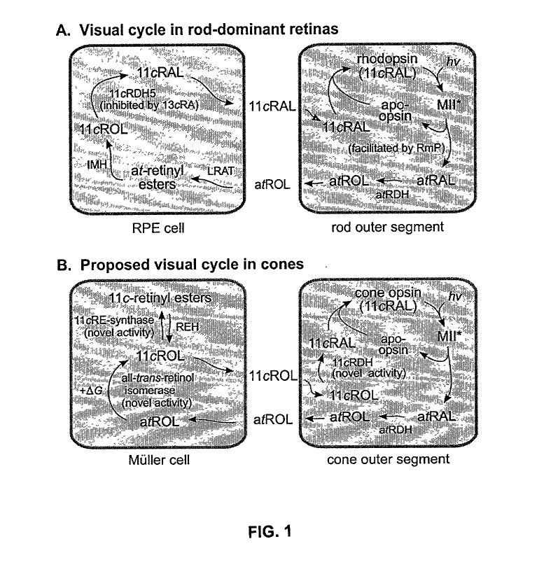

The vertebrate retina contains two types of photoreceptor cells. Rods are specialized for vision under low light conditions. Cones are less sensitive, provide vision at high temporal and spatial resolutions, and afford color perception. Under daylight conditions, the rod response is saturated and vision is mediated entirely by cones. Both cell types contain a structure called the outer segment comprising a stack of membranous discs. The reactions of visual transduction take place on the surfaces of these discs. The first step in vision is absorption of a photon by an opsin-pigment molecule, which involves 11-cis to all-trans isomerization of the retinaldehyde chromophore. Before light sensitivity can be regained, the resulting all-trans-retinaldehyde (atRAL) must dissociate from the opsin apoprotein and isomerize to 11-cis-retinaldehyde (11 cRAL). This recycling process, called the visual cycle, has been worked out primarily in rod-dominant retinas (Rando et al., 1992; Saari et al., 1994; Crouch et al., 1996). The visual cycle is diagramed in FIG. 1A.

A. Visual Pigment Regeneration

Several lines of evidence suggest that visual pigments may regenerate by a different mechanism in cones than in rods. Cones recover sensitivity following a photobleach much faster than do rods. In frog retinas separated from the RPE, cone opsins but not rhodopsin (rod pigment) regenerated spontaneously (Goldstein et al., 1967; Goldstein et al., 1973; Hood et al., 1973a; Hood et al., 1973b). After light exposure, isolated salamander cones but not rods recovered sensitivity with addition of 11-cis-retinol (11cROL) (Jones et al., 1989). Cultured Müller glial cells were shown to isomerize all-trans-retinol (atROL) to 11cROL, which they secreted into the medium (Das et al., 1992). Müller cells, in addition to RPE cells, contain cellular retinal-binding protein, which binds 11-cis-retinoids (Bunt-Milam et al., 1983). The latter two observations suggest cooperatively between cone photoreceptors and Müller cells in the recycling of visual chromophore. The cone-dominant chicken retina contains high levels of 11-cis-retinyl esters (11cRE), in contrast to virtually none in the rod-dominant bovine and rodent retinas (Rodriguez et al., 1989; Nicotra et al., 1994). The presence of 11cRE in cone-dominant retinas presupposes the enzymatic machinery for its processing. The final step in the visual cycle is oxidation of 11cROL by 11-cis-retinol dehydrogenase type-5 (11cRDH5). If the visual cycle in FIG. 1A subserved pigment regeneration for rods and cones, absence of 11cRDH5 would result in complete blindness. Instead, patients with recessive fundus albipunctatus, caused by mutations in the RDH5 gene, exhibit stationary night blindness with relatively normal cone function (Yamamoto et al., 1999; Gonzalez-Fernandez et al., 1999).

A similar phenotype of delayed rod dark-adaptation and relatively normal cone function is seen in 11cRDH knockout mice (Driessen et al., 2000). The sparing of cone vision with loss of 11cRDH5 argues for a cone form of 11cRDH. Finally, mice homozygous for a null mutation in the gene for Rpe65 have a defective rod visual-cycle, with no detectable rhodopsin in the retina and no cis-retinoids in the RPE (Redmond et al., 1998). Rpe65 may represent isomerohydrolase (IMH) in the rod visual cycle (Redmond et al., 1998) (FIG. 1A). However, cones in rpe65−/− mice contain normal visual pigments and appear to function normally. Collectively, these data suggest distinct pathways for the regeneration of visual pigments in rod and cone photoreceptors. The working model for an alternative visual cycle in cone photoreceptors is depicted in FIG. 1B. The purification and characterization of three new proteins that define catalytic steps in this pathway will be an important step in the understanding of the vision cycle in cones. The genes encoding these proteins will be strong candidates for inherited diseases that affect cone photoreceptors. These disease include the inherited macular degenerations and cone dystrophies. Currently, 15 genes for this group of diseases have been mapped but not yet cloned (http://www.sph.uth.tmc.edu/RetNet/).

B. Macular or Retinal Degeneration

As discussed above, macular degeneration (also referred to as retinal degeneration) is a disease of the eye that involves deterioration of the macula, the central portion of the retina. Approximately 85% to 90% of the cases of macular degeneration are the “dry” (atrophic or non-neovascular) type.

In “dry” macular degeneration, the deterioration of the retina is associated with the formation of small yellow deposits, known as drusen, under the macula. This phenomena leads to a thinning and drying out of the macula. The location and amount of thinning in the retinal caused by the drusen directly correlates to the amount of central vision loss. Degeneration of the pigmented layer of the retina and photoreceptors overlying drusen become atrophic and cause a slow of central vision. This often occurs over a decade or more.

Most people who lose vision from age related macular degeneration have “wet” macular degeneration (Bressler et al., 2000) In “wet” (neovascular) macular degeneration, abnormal blood vessels from the choroidal layer of the eye, known as subretinal neovascularization grow under the retina and macula. These blood vessels tend to proliferate with fibrous tissue, and bleed and leak fluid under the macula, causing the macula to bulge or move and distort the central vision. Acute vision loss occurs as transudate or hemorrhage accumulates in and beneath the retina. Permanent vision loss occurs as the outer retina becomes atrophic or replaced by fibrous tissues (Bressler et al., 1988).

C. Stargardt's Disease

Stargardt's disease (STGD) is a recessive form of macular degeneration with an onset during childhood (Allikmets et al., 1997; Lewis et al., 1999; Stone et al., 1998). STGD is characterized clinically by progressive loss of central vision and progressive atrophy of the retinal pigment epithelium (RPE) overlying the macula (Stargardt, 1909). Mutations in the human ABCR gene for RmP are responsible for STGD. Early in the disease course, patients show delayed dark adaptation but otherwise normal rod function (Fishman et al., 1991). Histologically, STGD is associated with deposition of lipofuscin pigment granules in RPE cells, presumably arising from impaired digestion after phagocytosis of shed distal outer-segments (Birnbach et al., 1994; De Laey and Verougstraete, 1995). Degeneration of the RPE occurs subsequently, with photoreceptor degeneration appearing late in the disease. This pathological picture has lead to the conclusion that STGD is primarily a defect of the RPE (Lee and Heckenlively, 1999). However, the pattern of early RPE degeneration and preservation of photoreceptors must be reconciled with the observation that RmP is present exclusively in outer segments and not expressed in RPE cells (Azarian and Travis, 1997).

Besides STGD, mutations in ABCR have been implicated in fundus flavimaculatus (Rozet et al., 1998), recessive retinitis pigmentosa (Cremers et al., 1998; Martinez-Mir et al., 1998), recessive cone-rod dystrophy (Cremers et al., 1998), and non-exudative age-related macular degeneration (AMD) (Allikmets et al., 1997; Lewis et al., 1999), although the prevalence of ABCR mutations in AMD is still uncertain (Stone et al., 1998). Similar to STGD, all four diseases are associated with delayed rod dark-adaptation (Alexander and Fishman, 1984; Fishman et al., 1991; Fishman et al., 1994; Steinmetz et al., 1993). Lipofuscin deposition in RPE cells is also seen prominently in AMD (Kliffen et al., 1997) and some cases of retinitis pigmentosa (Bergsma et al., 1977; Kolb and Gouras, 1974).

D. Detection of Macular or Retinal Degeneration

Identification of abnormal blood vessels in the eye can be done with an angiogram. This identification can help determine which patients are candidates for the use of a candidate substance or other treatment method to hinder or prevent further vision loss. Angiograms can also be useful for follow-up of treatment as well as for future evaluation of any new vessel growth.

A fluorescein angiogram (fluorescein angiography, fluorescein angioscopy) is a technique for the visualization of choroidal and retinal circulation at the back of the eye. Fluorescein dye is injected intravenously followed by multiframe photography (angiography) or ophthalmoscopic evaluation (angioscopy). Fluorescein angiograms are used in the evaluation of a wide range of retinal and choroidal diseases through the analysis of leakage or possible damage to the blood vessels that feed the retina. It has also been used to evaluate abnormalities of the optic nerve and iris by Berkow et al. (1984).

Similarly, angiograms using indocyanine green can be used for the visualization circulation at the back of the eye. Wherein fluorescein is more efficient for studying retinal circulation, indocyanine is better for observing the deeper choroidal blood vessel layer. The use of indocyanine angiography is helpful when neovascularization may not be observed with fluorescein dye alone.

II. Dehydrogenases

Dehydrogenases are enzyme that catalyzes the removal and transfer of hydrogen from a substrate in an oxidation-reduction reaction. A sub-group of dehydrogenases of particular interest in the current invention, short chain dehydrogenases, are enzymes with similar coenzyme-binding domains that perform oxidation-reduction reactions on short-chain alcohols and aldehydes. The short chain dehydrogenase superfamily can be described as belonging to the short chain alcohol dehydrogenase superfamily (SCAD) or the short chain dehydrogenase/reductase superfamily; these terms are used interchangeably throughout this disclosure. Short chain dehydrogenases include, but are not limited to a 11-cis-retinol dehydrogenase such as GenBank Accession number Q92781, identified as SEQ ID NO:2 and the nucleic acid encoding the enzyme such as GenBank Accession number U43559, identified as SEQ ID NO: 1; a novel 11-cis-retinol dehydrogenase isolated from bovine retinal pigment epithelium is identified as SEQ ID NO: 4, with the nucleic acid sequence encoding the enzyme identified as SEQ ID NO: 3; a retinol dehydrogenase type I, II, or III, such as GenBank Accession numbers U18762, U33500, U33501, identified as SEQ ID NOS: 6, 8, and 10 respectively and the nucleic acid encoding the type I, II and III enzymes identified as SEQ ID NOS: 5, 7, and 9 respectively; a cis retinol androgen dehydrogenase such as GenBank Accession number AF030513, identified as SEQ ID NO: 12 and the nucleic acid encoding the enzyme identified as SEQ ID NO: 11; an all-trans-retinol dehydrogenase such as a homo sapien or bos taurus photoreceptor outer segment all-trans-retinol dehydrogenase given as GenBank Accession numbers AF229845, and AF229846, identified as SEQ ID NOS: 14 and 16 respectively and the nucleic acid encoding the enzymes identified as SEQ ID NOS: 13 and 15 respectively; a β-hydroxy butyrate dehydrogenase such as GenBank Accession number Q02338, identified as SEQ ID NO: 18 and a nucleic acid encoding a β-hydroxy butyrate dehydrogenase such as GenBank Accession number BE503838 identified as SEQ ID NO: 17; a short chain dehydrogenase retSDR1 (Haeseleer et al., 1998) such as GenBank Accession number AF061741, identified as SEQ ID NO: 20 and the nucleic acid encoding the enzyme identified as SEQ ID NO: 19; a 15-hydroyxprostaglandin dehydrogenase such as GenBank Accession number NM_{—}000860, identified as SEQ ID NO: 22 and the nucleic acid encoding the enzyme identified as SEQ ID NO: 21; 17β-hydroxysteroid dehydrogenases (Su et al., 1999), and the nucleic acid encoding the enzyme such as GenBank Accession number L40802 identified as SEQ ID NO: 23; and a cRDH that catalyzes the oxidation of 9-cis- but not all-trans-retinol characterized by Gamble et al. (1999). These proteins are further described by Persson et al. (1991), herein incorporated by reference.

Dehydrogenases are either cytosolic or membrane-bound enzymes that utilize a large number of substrates, including steroids and prostaglandins. The dehydrogenase enzymes will oxidize or reduce their substrates, respectively, depending on whether the cofactor is reduced or oxidized (i.e., SCADs are oxidoreductases). Thus, it would not be surprising to find that other retinol dehydrogenases belong to the short chain dehydrogenase superfamily. For example, the enzyme that reduces all-trans retinal to all-trans retinol in the photoreceptors after bleaching of the visual pigments may also be a short chain dehydrogenase (Bliss et al., 1948). It is also possible that dehydrogenases that are not members of the short chain dehydrogenase family are able to recognize 11-cis-retinol and oxidize it to 11-cis-retinaldehyde. These dehydrogenases would most probably be associated with the ocular area, and could be used in the screening method of the current invention.

A. 11-cis-retinol Dehydrogenase

11-cis-retinol dehydrogenase (11cRD) is structurally related to several previously sequenced proteins belonging to the short chain dehydrogenase superfamily (Persson, et al., 1991) which can oxidize 11 cis-retinol to 11-cis-retinaldehyde. Simon et al. (1995) first reported that the membrane-bound 11-cis-retinol dehydrogenase belonging to the SCAD superfamily. 11cRD is involved in the final step in the visual cycle where 11-cis-retinol dehydrogenase type-5 (11cRDH5) oxidized 11cROL. 11cRD is closely related to a mitochondrial matrix dehydrogenase, the D-hydroxybutyrate dehydrogenase (Churchill et al., 1992); it also is similar to other proteins including the 3-oxoacyl[acyl carrier protein]reductase from Escherichia coli (Rawlings et al., 1992) and the human estradiol 17-dehydrogenase (Luu-The et al., 1989; Peltoketo et al., 1988). The first highly conserved region in 11cRD, involves the residues 63-69, and having the conserved motif G-X-X-X-G-X-G is thought to be the cofactor binding site for NAD, NADP, or the reduced forms. The sequence motif Y-X-X-X-K, thought to be part of the active site, is the most highly conserved motif in SCADs (Persson, et al., 1991; Simon et al., 1995). It is a conceived that any composition or methods described herein using 11cRDH5 or 11cRD may be used with any other member of the short chain dehydrogenase superfamily.

A novel 11-cis-retinol dehydrogenase (SEQ ID NO: 4) was cloned from bovine retinal pigment epithelium and has been shown to recognize and convert 11 cis-retinol to 11-cis-retinaldehyde. This enzyme can be used in a screening assay for the treatment of macular or retinal degeneration.

B. Assays for the Dehydrogenase Activity

A variety of assays can be used to obtain the activity of the dehydrogenase, including the assays known in the art for proteins and nucleic acids. The activity can be determined by obtaining information on reactants and products, such as assaying to see if 11-cis-retinol has been oxidized to 11-cis-retinaldehyde or if NAD⁺ has been reduced to NADH. Assays can include chromatographic techniques such as normal or reverse phase HPLC, liquid chromatography, capillary chromatography, affinity chromatography or supercritical flow chromatography (Freifelder, 1982); mass spectrometry techniques such as fast-atom bombardment (FAB) mass spectrometry; time of flight (TOF) mass spectrometry; gas chromatography coupled to mass spectrometry; optical techniques such as infrared, fluorescence or Raman spectroscopy. Techniques that allow for the rapid or simultaneous analysis of dehydrogenase activity are also contemplated, including microfluidics or lab-on-a-chip techniques which involve automated fluid movement for separation and detection on a platform using microcolumns, capillaries or reaction chambers are contemplated.

Activity can be assayed by analyzing the product, NADH, electrochemically. A redox coupling agent may also be added to the NADH to form an electroactive coupling agent which can be detected at a lower voltage than the product alone (U.S. Pat. No. 5,240,571). Other analysis techniques include photometric detection of NADH. Various pretreatment steps may be required before photometric detection, including removing the red blood cells for colorimetric detection or removing excessive amounts of protein, lipid, bilirubin or hemoglobin that can interfere with certain photometric detection systems. Radiochemical assay is a preferred method of determining activity, and involves, for example, the transfer of a tritiated label from NAD⁺ to NADH. Further assays for determining the enzymatic activity of 11cRD are described by, for example, by Saari et al. (1993; 2000) which are herein incorporated by reference.

III. ABCR Knockout Mice

An important aspect of the animal models of the present invention is a null mutation in the abcr gene for RmP. One method of inhibiting the expression of the abcr gene in an animal is to disrupt the gene in germline cells and produce offspring from these cells. This method is generally known as knockout technology.

In a general sense, preparation of a knockout mammal requires first introducing a nucleic acid construct that will be used to suppress expression of a particular gene into an undifferentiated cell type termed an embryonic stem (ES) cell. This cell is then injected into a mammalian embryo, where it hopefully will be integrated into the developing embryo. The embryo is then implanted into a foster mother for the duration of gestation.

U.S. Pat. No. 5,616,491, incorporated herein by reference in its entirety, generally describes the techniques involved in the preparation of knockout mice, and in particular describes mice having a suppressed level of expression of the gene encoding CD28 on T cells, and mice wherein the expression of the gene encoding CD45 is suppressed on B cells. Pfeffer et al. (1993) describe mice in which the gene encoding the tumor necrosis factor receptor p55 has been suppressed. The mice showed a decreased response to tumor necrosis factor signaling. Fung-Leung et al. (1991a; 1991b) describe knockout mice lacking expression of the gene encoding CD8. These mice were found to have a decreased level of cytotoxic T cell response to various antigens and to certain viral pathogens such as lymphocytic choriomeningitis virus.

The term “knockout” refers to a partial or complete suppression of the expression of at least a portion of a protein encoded by an endogenous DNA sequence in a cell. The term “knockout construct” refers to a nucleic acid sequence that is designed to decrease or suppress expression of a protein encoded by endogenous DNA sequences in a cell. The nucleic acid sequence used as the knockout construct is typically comprised of: (1) DNA from some portion of the gene (exon sequence, intron sequence, and/or promoter sequence) to be suppressed; and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA).

The knockout construct nucleic acid sequence may comprise (1) a full or partial sequence of one or more exons and/or introns of the gene to be suppressed, (2) a full or partial promoter sequence of the gene to be suppressed, or (3) combinations thereof. Typically, the knockout construct is inserted into an embryonic stem cell (ES cell) and is integrated into the ES cell genomic DNA, usually by the process of homologous recombination. This ES cell is then injected into, and integrates with, the developing embryo.

The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, many progeny of the cell will no longer express the gene at least in some cells, or will express it at a decreased level, as the DNA is now disrupted by the antibiotic resistance gene.

Usually, the DNA to be used in the knockout construct will be one or more exon and/or intron regions, and/or a promoter region from the genomic sequence provided herein, but may also be cDNA sequence. Generally, the DNA will be at least about 500 bp to 1 kilobase (kb) in length, and in certain aspects up to 3-4 kb in length, thereby providing sufficient complementary sequence for hybridization when the knockout construct is introduced into the genomic DNA of the ES cell.

ABCR encodes rim protein (RmP), an ATP-binding cassette (ABC) transporter in the outer-segment discs of rod and cone photoreceptors (Papermaster et al., 1978; Molday et al., 2000; Azarian et al., 1997). The transported substrate for RmP is unknown. Mice generated with a knockout mutation in the abcr gene (Weng et al., 1999) are useful for the study of RmP function as well as for an in vivo screening of the effectiveness for candidate substances. These animals manifested the complex ocular phenotype: (i) slow photoreceptor degeneration, (ii) delayed recovery of rod sensitivity following light exposure, (iii) elevated atRAL and reduced atROL in photoreceptor outer-segments following a photobleach, (iv) constitutively elevated phosphatidylethanolamine (PE) in outer-segments, and (v) accumulation of lipofuscin in RPE cells (Weng et al., 1999). Based on these data and the results of reconstitution experiments (Sun et al., 1999; Ahn et al., 2000), it has been suggested that RmP may function as a flippase for N-retinylidene-phosphatidylethanolamine (APE), the normally occurring Schiff-base conjugate of PE and atRAL. A potential role for RmP may be to accelerate recovery of rod sensitivity following light exposure by removing conjugated atRAL from the interior of outer-segment discs (Weng et al., 1999).

Rim protein (RmP) is an ABC transporter in rod outer-segment discs. The human gene for RmP (ABCR) is affected in several recessive retinal degenerations. RmP is a glycoprotein of 210 kDa in the rims of outer-segment discs (Papermaster et al., 1978). Outer segments comprise a stack of these flattened membranous structures, which are the sites of photon-capture and the reactions of visual transduction. In rods, discs are topologically separate from one another and the plasma membrane. Recently, RmP was purified from bovine retinas, and shown to be expressed exclusively in rod outer-segment discs (Azarian and Travis, 1997; Illing et al., 1997). Sequence analysis revealed it to be a new member of the ATP binding-cassette (ABC) transporter family. Most ABC transporters effect ATP-dependent translocation of specific substrates across cellular membranes. The translocated substrate for RmP is unknown.

The generation and phenotypic characterization of mice with a null mutation in the abcr gene for RmP is an aspect of the current invention. These mice can be used for in vivo screening of candidate substances in there effect on the activity of 11cRD. The mice show a delayed rod dark-adaptation; a transient accumulation of all-trans-RAL and transient depletion of all-trans-ROL and all-trans-RE following a photobleach; a 1.6-fold increased PE in outer segments; a presence of protonated and absence of non-protonated N-retinylidene-PE in outer segments; and a dramatically increased A2-E in RPE cells.

A. Generation of abcr−/− Mice That Lack RmP

A targeting construct was transfected into 129-strain embryonic stem (ES) cells, and a targeting frequency of 2% was observed in clones that survived G418 and gancyclovir selection. Blastocyst injection of six clones resulted in the birth of 17 high-percentage male chimeras. Southern blot analysis of tail-cut DNA from their F1 offspring revealed germ-line transfer of the disrupted locus in three lines, representing three independent targeting events. The Bam HI fragments detected in lines abcr-2 and abcr-3 mice were larger than the predicted 18 kB, suggesting tandem integration of the construct. Heterozygous sibs from all three lines were inbred to yield abcr−/− homozygotes.

Generation of abcr−/− mice is described in detail by Weng et al. (1999), which is herein incorporated by reference. A replacement-type targeting construct for abcr was assembled by screening a mouse-strain 129Sv/J lambda genomic library (Stratagene) with a probe from the 5′-region of a mouse RmP cDNA (Azarian and Travis, 1997). A clone of ˜18 kb containing the first four exons and promoter region of abcr was isolated, restriction mapped, and partially sequenced. A 3.2-kb Eco RI/Bam HI fragment from upstream of abcr, and a 4.2-kb Xba I/Sac I fragment from intron I were placed in pBluescript, flanking a 1.7-kb Sal I/Xho I fragment of pPGKneobpA containing the neomycin phosphotransferase gene (Neo^{r}) under control of the mouse PGK-promoter. Successful targeting with this construct would result in deletion of a 4.0-kb fragment of abcr containing the promoter and first protein-coding exon (Azarian et al., 1998). Finally, a 2.2 kb Sal I/Xho I fragment pMC1tkbpA, containing the Herpes Simplex virus thymidine kinase gene (HSV-TK), was cloned into the upstream Sal I-site of the targeting vector for negative selection. The construct was electroporated into J1 ES cells derived from 129Sv mice. ES cell clones resistant to both G418 and gancyclovir were expanded and analyzed by PCR. After sequencing the diagnostic 3.6- and 5.1-kb amplification products, cells from six clones that had undergone homologous recombination were injected into C57BL/6 blastocysts, resulting in the birth of 17 high-percentage chimeras by coat color. Six male chimeras were bred with C57BL/6 females to check for germline transmission. Three germline-transmitting animals, representing three independent targeting events, were bred with 129SVEV females. Tail-cut DNA from the offspring was analyzed by Southern blot analysis. Heterozygotes from each line were inbred to yield abcr−/− homozygous mutants.

The expression of the disrupted gene by nuclease protection analysis of RNA from wild-type and abcr−/− retinas was tested. In contrast to samples from control retinas, no transcription products were detected in retina from mice of the three abcr−/− lines. To test for protein expression, immunoblot analysis was performed on retinal homogenates from wild-type, abcr+/−, and abcr−/− mice from all three lines. No RmP immunoreactivity was detected in samples from abcr−/−, and partial immunoreactivity was detected in samples from abcr+/− mutants. The same retinal homogenates were analyzed with antisera against the unrelated outer-segment proteins: rds/peripherin, rom1, and rhodopsin. No difference in the levels of these proteins was observed between wild-type and abcr−/− mice. Similarly, there are no difference in the patterns of total protein from wild-type and abcr−/− outer-segments except for absence of the 210-kDa RmP band in the mutant. These results that the abcr gene was knocked-out and that loss of RmP does not affect the overall protein-composition of outer segments.

B. Delayed Dark Adaptation

All vertebrates sustain a period of reduced visual sensitivity following light exposure, due to the accumulation of an undefined activating photoproduct (reviewed in Fain et al., 1996; Leibrock et al., 1998). Prolongation of this reduced-sensitivity state in abcr−/− mutants is similar to the clinical pattern in patients with the ABCR-mediated retinal diseases including STGD (Alexander and Fishman, 1984; Fishman et al., 1991; Fishman et al., 1994; Steinmetz et al., 1993). During the first 40 min in darkness following a photobleach, rod cells in abcr−/− mice behave as if exposed to low-level background illumination. The otherwise normal ERG parameters in dark-adapted abcr−/− mice suggest that RmP plays no direct role in visual transduction. Opsin apoprotein has been shown to interact spontaneously with all-trans-RAL to form a non-covalent complex that activates the visual-transduction pathway with approximately 10⁶-fold greater efficiency than opsin alone, and nearly 10% the efficiency of photoactivated rhodopsin (metarhodopsin II) (Jager et al., 1996; Melia et al., 1997; Surya et al., 1995). A likely explanation for the delayed dark adaptation in abcr−/− mice is transient accumulation of this “noisy” opsin/all-trans-RAL photoproduct. As rhodopsin regenerates, all-trans-RAL is displaced by the formation of a covalent complex between 11-cis-RAL and opsin, with return of rod cells to normal dark-sensitivity. The time-courses following a photobleach for the delay in dark adaptation, disappearance of all-trans-RAL, and regeneration of rhodopsin are consistent with this explanation. The delayed dark adaptation in humans with ABCR-mediated retinal disease also may result from transient accumulation in outer segments of the opsin/all-trans-RAL product.

C. Abnormal Phospholipid Composition

Another aspect of the abcr−/− phenotype is a 1.6-fold increase in outer-segment PE. This represents a large difference in PE mole-percentage (54 mol % in abcr−/− versus 34 mol % in wild-type outer segments) according to published values (Fliesler and Anderson, 1983). Phospholipids, especially PE, are rapidly turned-over in outer segments by the action of transfer proteins, phospholipases, and acyltransferases. Based on these observations, the turnover of PE may be reduced in outer segments from abcr−/− mice. Phospholipids are normally distributed asymmetrically across the disc bilayer, with 70-80% of PE present in the outer (cytoplasmic) leaflet (Miljanich et al., 1981; Wu and Hubbell, 1993). In abcr−/− mutants, the distribution of PE may be more symmetrical, with the excess located preferentially in the inner (intradiscal) leaflet. This is understandable, since the enzymes that effect phospholipid-turnover are present in the cytoplasmic compartment, and hence can only act on PE oriented with its headgroup on the extradiscal surface.

D. Assaying Dehydrogenase Activities or Amounts

The effect of a therapeutic agent in knockout mice can be evaluated by determining the amount of lipofuscin or a component of lipofuscin such as A2-E. A2-E and iso-A2-E can be extracted from RPE tissue using known procedures, such as those described by Reinboth et al. (1997). The A2-E can then be analyzed by, for example, HPLC with a preferable detection wavelength of 430 nm. Authentic A2-E standards can be synthesized (Parish et al., 1998), and used to identify and quantify A2-E in tissue-sample extracts. Iso-A2-E is generated by exposure of A2-E to room light, and purified by HPLC. Confirmation of synthetic and endogenous A2-E can be achieved by fast-atom bombardment mass-spectrometry, and UV-vis spectroscopy.

The concentration of N-retinylidene-PE is a likely precursor of A2-E (Parish et al., 1998) can also be analyzed. N-retinylidene-PE isoforms are extracted from outer segments and analyzed by HPLC with detection wavelengths of 205 nm for phospholipids, 360 nm for unprotonated N-retinylidene-PE, and 450 nm for protonated N-retinylidene-PE were used. To confirm the presence of a Schiff base in the N-retinylidene-PE fractions, eluted peaks can be treated with concentrated HCl or 2% KOH in methanol, and the samples re-analyzed by HPLC.

Other assaying techniques for the effect of a therapeutic agent in knockout mice are contemplated, and include direct observation using a light microscope or observation using an electron microscope. The addition of a fluorescent dye such as fluorescein is contemplated. Other assaying techniques that are known in the art and discussed herein are also contemplated for the determination of dehydrogenase activity.

IV. Mechanism of ABCR-mediated Disease

An important pathological feature of both STGD and AMD is the accelerated accumulation of lipofuscin in RPE phagosomes (De Lacy and Verougstraete, 1995; Kliffen et al., 1997). The major fluorescent species of lipofuscin is N-retinylidene-N-retinylethanolamine (A2-E), a positively charged Schiff-base condensation-product of two retinaldehydes with ethanolamine (Eldred and Lasky, 1993). The closely related species, iso-A2-E, contains one 13-cis isomer of retinaldehyde. Iso-A2-E probably arises by photoisomerization of A2-E rather than direct condensation of ethanolamine with 13-cis-RAL, since this retinoid species is present in vivo at very low levels (Parish et al., 1998). The chromatographic and spectral properties of A2-E and iso-A2-E are well defined (Eldred and Katz, 1988; Parish et al., 1998), with a λₘₐₓ for each of approximately 430 nm. Since lipofuscin accumulation is a feature of several human ABCR-mediated diseases, A2-E and iso-A2-E concentrations were determined in retina, RPE, and purified outer segments from wild-type and abcr−/− mice by HPLC. RPE extracts from 16 to 20-week-old abcr−/− mice contained 21 and 10 pmoles per eye of A2-E and iso-A2-E, respectively. RPE from age-matched control mice had <1 pmole per eye of each isomer. No A2-E or iso-A2-E was detected in retinas or purified outer segments from wild-type or abcr−/− mice. A2-E by synthesizing it in vitro (Parish et al., 1998) has identical chromatographic and spectral properties compared with those of A2-E isolated from mouse RPE. Fast-atom bombardment mass-spectrometry was performed synthetic and endogenous A2-E. Both yielded a molecular-ion (m/z) peak of 592.4, which is in good agreement with the calculated molecular mass of 592.45 for C₄₂H₅₈ON.

Ultrastructural analysis of eyes from 44-week-old mice showed significant accumulation of dense bodies within RPE cells of abcr−/− mutants. These included large oval structures of high electron density, presumably representing melanosomes or melanosome-phagosome fusion particles (Feeney-Bums and Eldred, 1983). Numerous smaller structures of variable density, probably representing lipofuscin granules are also present. In many cells, the dense bodies were displaced into the apical processes. Thickening of Bruch's membrane, between the RPE and choroid is also observed in abcr−/− mice. The photoreceptors and rest-of-retina in abcr−/− mutants were indistinguishable from those in wild-type mice (not shown). In particular, both rod and cone outer-segments were of normal dimensions and showed good alignment of discs.

N-retinylidene-PE, a likely precursor of A2-E (Parish et al., 1998), was present at approximately equal levels in outer segments from dark-adapted wild-type and abcr−/− mice. This was expected, since the dark-adapted levels of all-trans-RAL were similar between wild-type and mutant retinas, and approximately 100% of all-trans-RAL was present as N-retinylidene-PE. However, N-retinylidene-PE was largely protonated in abcr−/− mutants and unprotonated in wild-type mice. Upon shedding of rod outer-segments, the accumulated N-retinylidene-PE is sequestered into RPE phagolysosomes where the low-pH environment favors the formation of A2-E salts (Eldred and Lasky, 1993). At low concentrations, A2-E inhibits normal proteolysis in lysosomes (Eldred, 1995; Holz et al., 1999). At higher concentrations, A2-E may act as a positively charged lysosomotropic detergent, dissolving cellular membranes and ultimately killing the RPE cell (Eldred and Lasky, 1993). Accumulations of electron-dense bodies in RPE cells and significant thickening of Bruch's membrane in abcr−/− mice were observed by electron microscopy (Weng et al., 1999). A similar ultrastructural pattern has been described in RPE from humans with early-stage STGD (Birnbach et al., 1994; Steinmetz et al., 1991). These ultrastructural changes are consistent with the observed accumulation of A2-E in RPE cells, and indicate significant lipofuscin deposition. There was no evidence of RPE cell-enlargement or degeneration, pathologic features of advanced STGD (Birnbach et al., 1994; Eagle et al., 1980). Photoreceptor degeneration also occurred slowly in abcr−/− mice. In the ABCR-mediated retinal dystrophies, inventors suggest, but are not confined by the hypothesis, that photoreceptor degeneration follows the death of RPE cells, and results from loss of the RPE support-role (reviewed in Steinberg, 1985). This explains the slower photoreceptor degeneration in abcr−/− mice compared with the other retinal mutants that act by photoreceptor cell-autonomous mechanisms.

Another hallmark of STGD and AMD is striking early involvement of the central retina, or macula. The perifoveal region of the human macula contains a ring-like area of very high rod-density (150,000 mm⁻² compared to ˜30,000 mm⁻² in peripheral retina) (Jonas et al., 1992). The fovea itself is populated exclusively with cone photoreceptors. Selective involvement of the macula in STGD may result from the high ratio of rod outer-segments to RPE cells in this region. Ring-like deficits in the central visual field, and ring-like areas of higher lipofuscin-deposition in RPE overlying the macula have been observed in humans (Lee and Heckenlively, 1999; Wing et al., 1978). Thus, foveal cones probably do not contribute A2-E precursor to the macular RPE in the ABCR mediated diseases. Subsequent degeneration of the foveal cones with loss of central vision may result from rod-mediated “poisoning” of the macular RPE. The peripheral retina is affected later in STGD due to the more favorable ratio of outer-segments to RPE-cells. Mouse retinas do not contain a macula, more closely resembling a human peripheral retina (Carter-Dawson and LaVail, 1979). In studies of two pedigrees, the ABCR null-phenotype in humans was retinitis pigmentosa (Cremers et al., 1998; Martinez-Mir et al., 1998), a blinding disease that predominantly affects the peripheral retina. Thus, early involvement of the less-vulnerable peripheral retina may only be seen with more severe alleles of ABCR.

V. Model for the Function of RmP

RmP plays an important role in macular and retinal degeneration. Mutations in the human ABCR gene for RmP are responsible for STGD. A model of RmP function suggests that RmP functions as an outwardly directed flippase for protonated N-retinylidene-PE. Such a role is consistent with the biology of ABC transporters, since other members of this family are also phospholipid flippases including MDR2 in mammals (Smit et al., 1993), Yor1p in yeast (Decottignies et al., 1998), MsbA in E. coli (Zhou et al., 1998); and CDR1 in C. albicans (Dogra et al., 1999). Recently, it was shown that several retinoids, including all-trans-RAL, increased the ATPase activity of reconstituted RmP (Sun et al., 1999). However, this increase in ATPase activity was only seen when RmP was reconstituted in the presence of PE, suggesting that all-trans-RAL reacted with PE to form N-retinylidene-PE, the putative substrate for RmP. The proposed N-retinylidene-PE flippase activity of RmP may serve two potential functions.

First, it may accelerate dark adaptation by speeding the transfer of all-trans-RAL from the disc interior to the cytoplasmic surface following a photobleach. Although it has not been established on which surface of the membrane 11-cis-RAL and all-trans-RAL interact with opsin, for the other seven-transmembrane receptors, ligands arrive and depart via the extracellular face. Since the disc interior is topologically equivalent to the extracellular space, it is likely that all-trans-RAL dissociates from opsin into the intradiscal space. The elevated levels of all-trans-RAL and reduced levels of all-trans-ROL and all-trans-retinyl esters following a photobleach in abcr−/− mutants suggest that RmP functions in a step following photoactivation of rhodopsin but preceding reduction of all-trans-RAL. Therefore, all-trans-retinol dehydrogenase must act downstream of RmP. Localization of RmP to the disc rim immediately subjacent to the plasma membrane may enhance the efficiency of retinoid recycling by shortening the diffusion path to the RPE. There is substantial accumulation of retinyl ester above the dark-adapted level in wild-type RPE cells, suggesting significant basal synthesis in response to light exposure. The observed difference in retinyl-ester levels between RPE from wild-type and mutant mice after light-exposure may reflect impaired ester synthesis due to reduced apical uptake of all-trans-ROL. On the other hand, this difference may reflect “sickness” of the RPE due to lipofuscin accumulation. Mutant mice lacking RmP regenerate rhodopsin normally and achieve full dark-adaptation, albeit more slowly than in wild-type mice. Thus, an alternative path must exist for the recycling of all-trans-RAL, probably involving free diffusion through disc membranes (Groenendijk et al., 1984; Jin et al., 1994). RmP may accelerate the removal of all-trans-RAL from the disc interior by the translocation of N-retinylidene-PE. In cone outer-segments, the intradiscal and extracellular spaces are contiguous, which may explain the absence of RmP from these cells (Allikmets et al., 1997; Illing et al., 1997; Sun and Nathans, 1997). Also, cones are not used for vision under low-light conditions and thus have no requirement for rapid dark-adaptation.

The second and perhaps more important function of RmP as a flippase may be to eliminate N-retinylidene-PE from the disc interior, thus protecting the RPE from lipofuscin accumulation. Following a significant photobleach, all-trans-RAL is present at very high concentrations within the disc, favoring formation of N-retinylidene-PE. In abcr−/− mutants lacking the RmP-flippase activity, protonated N-retinylidene-PE may be “entombed” within the disc interior. In normal animals, protonated N-retinylidene-PE is translocated to the exterior (cytoplasmic) surface of the disc. Reduction of all-trans-RAL and subsequent esterification of the resulting alcohol shifts the equilibrium to favor hydrolysis of the N-retinylidene-PE Schiff-base.

According to the model for RmP function described herein, photoreceptor degeneration and visual loss in the ABCR-mediated diseases is a three-step process. First, protonated N-retinylidene-PE accumulates in outer segments due to loss of the flippase-activity. Second, isomers of A2-E build-up in RPE lysosomes, resulting in progressively impaired digestion of phagocytosed outer segments and ultimate dissolution of cellular membranes. Finally, photoreceptors die due to loss of RPE-support functions. Since all-trans-RAL is only released following light activation of rhodopsin, a strong prediction of this model is that A2-E accumulation should be reduced or abolished in abcr−/− mice raised in total darkness. A2-E has been found to be virtually absent from the RPE of dark-reared abcr−/− mice. A corollary of this prediction is that patients with STGD may slow the progression of their disease by avoiding bright light. The etiology of AMD may be similar to that of STGD, with lipofuscin accumulation in RPE cells causing secondary photoreceptor degeneration. Accordingly, the abcr−/− knockout mouse is a good animal model for AMD, independent of its genetic cause.

VI. Nucleic Acid Compositions

Certain embodiments of the present invention involve the synthesis and/or mutation of at least one isolated DNA segments and recombinant vectors encoding one or more dehydrogenase such as those shown in SEQ ID NO: 2, 4, 6, 8, 10, 12, 14, 16, 18, 20, and 22, or one or more inhibitors of a dehydrogenase. Embodiments of the invention also involve the creation and use of recombinant host cells through the application of DNA technology, that express one or more dehydrogenase polypeptides. In certain aspects, a nucleic acid encoding a dehydrogenase or an inhibitor of a dehydrogenase comprises a wild-type or a mutant nucleic acid. The nucleic acid compositions can, for example, be used in an assay for 11cRD activity.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

A. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table non-limiting, purine and pyrimidine derivatives and analogs is also provided herein below.

TABLE 1
Purine and Pyrmidine Derivatives or Analogs

| Abbr. | Modified base description | Abbr. | Modified base description |
|---|---|---|---|
| ac4c | 4-acetylcytidine | Mam5s2u | 5-methoxyaminomethyl-2-thiouridine |
| Chm5u | 5-(carboxyhydroxylmethyl) uridine | Man q | Beta,D-mannosylqueosine |
| Cm | 2′-O-methylcytidine | Mcm5s2u | 5-methoxycarbonylmethyl-2-thiouridine |
| Cmnm5s2u | 5-carboxymethylamino-methyl-2-thiouridine | Mcm5u | 5-methoxycarbonylmethyluridine |
| Cmnm5u | 5-carboxymethylaminomethyluridine | Mo5u | 5-methoxyuridine |
| D | Dihydrouridine | Ms2i6a | 2-methylthio-N6-isopentenyladenosine |
| Fm | 2′-O-methylpseudouridine | Ms2t6a | N-((9-beta-D-ribofuranosyl-2-methylthiopurine-6- |
| | | | yl)carbamoyl)threonine |
| Gal q | Beta,D-galactosylqueosine | Mt6a | N-((9-beta-D-ribofuranosylpurine-6-yl)N-methyl- |
| | | | carbamoyl)threonine |
| Gm | 2′-O-methylguanosine | Mv | Uridine-5-oxyacetic acid methylester |
| I | Inosine | o5u | Uridine-5-oxyacetic acid (v) |
| I6a | N6-isopentenyladenosine | Osyw | Wybutoxosine |
| m1a | 1-methyladenosine | P | Pseudouridine |
| m1f | 1-methylpseudouridine | Q | Queosine |
| m1g | l-methylguanosine | s2c | 2-thiocytidine |
| m1I | 1-methylinosine | s2t | 5-methyl-2-thiouridine |
| m22g | 2,2-dimethylguanosine | s2u | 2-thiouridine |
| m2a | 2-methyladenosine | s4u | 4-thiouridine |
| m2g | 2-methylguanosine | T | 5-methyluridine |
| m3c | 3-methylcytidine | t6a | N-((9-beta-D-ribofuranosylpurine-6-yl)carbamoyl)threonine |
| m5c | 5-methylcytidine | Tm | 2′-O-methyl-5-methyluridine |
| m6a | N6-methyladenosine | Um | 2′-O-methyluridine |
| m7g | 7-methylguanosine | Yw | Wybutosine |
| Mam5u | 5-methylaminomethyluridine | X | 3-(3-amino-3-carboxypropyl)uridine, (acp3)u |

A nucleobase may be comprised in a nucleoside or nucleotide, using any chemical or natural synthesis method described herein or known to one of ordinary skill in the art.

B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

C. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

D. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ intemucleotide linkages; U.S. Pat. No. 5,714,606 which describes a modified intemucleotide linkage wherein a 3′-position oxygen of the intemucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moiety which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide intemucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moiety replacing phosphodiester backbone moiety used for improved nuclease resistance, cellular uptake and regulating RNA expression; U.S. Pat. No. 5,858,988 which describes hydrophobic carrier agent attached to the 2′-O position of oligonucleotides to enhanced their membrane permeability and stability; U.S. Pat. No. 5,214,136 which describes oligonucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

In a non-limiting example, one or more nucleic acid analogs may be prepared containing about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges).

E. Polyether and Peptide Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moiety that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR, to reduce false positives and discriminate between single base mutants, as described in U.S. Pat. No. 5891,625. Other modifications and uses of nucleic acid analogs are known in the art, and are encompassed by the nucleic acid encoding for dehydrogenases. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. U.S. application Ser. No. 117,363 describes several alkylamino moieties used to enhance cellular uptake of a PNA. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

F. Antisense and Ribozymes

Modulators of dehydrogenases include molecules that directly affect RNA transcripts encoding dehydrogenase polypeptides. Antisense and ribozyme molecules target a particular sequence to achieve a reduction or elimination of a particular polypeptide, such as dehydrogenases. Thus, it is contemplated that nucleic acid molecules that are identical or complementary to all or part of SEQ ID NO:1, 3, 5, 7, 9, 11, 13, 15, 17, 19, 21 and 23 are included as part of the invention.

a. Antisense Molecules

Antisense methodology takes advantage of the fact that nucleic acids tend to pair with “complementary” sequences. By complementary, it is meant that polynucleotides are those which are capable of base-pairing according to the standard Watson-Crick complementarily rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs may include regions complementary to intron/exon splice junctions. Thus, antisense constructs with complementarily to regions within 50-200 bases of an intron-exon splice junction may be used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

b. Ribozymes

The use of dehydrogenase-specific ribozymes is claimed in the present application. The following information is provided in order to compliment the earlier section and to assist those of skill in the art in this endeavor.

Ribozymes are RNA-protein complexes that cleave nucleic acids in a site-specific fashion. Ribozymes have specific catalytic domains that possess endonuclease activity (Kim and Cech, 1987; Gerlack et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarveret al., 1990; Sioudet al., 1992). Recently, it was reported that ribozymes elicited genetic changes in some cell lines to which they were applied; the altered genes included the oncogenes H-ras, c-fos and genes of HIV. Most of this work involved the modification of a target mRNA, based on a specific mutant codon that is cleaved by a specific ribozyme. In light of the information included herein and the knowledge of one of ordinary skill in the art, the preparation and use of additional ribozymes that are specifically targeted to a given gene will now be straightforward.

Several different ribozyme motifs have been described with RNA cleavage activity (reviewed in Symons, 1992). Examples that would be expected to function equivalently for the down regulation of dehydrogenases include sequences from the Group I self splicing introns including tobacco ringspot virus (Prody et al., 1986), avocado sunblotch viroid (Palukaitis et al., 1979; Symons, 1981), and Lucerne transient streak virus (Forster and Symons, 1987). Sequences from these and related viruses are referred to as hammerhead ribozymes based on a predicted folded secondary structure.

Other suitable ribozymes include sequences from RNase P with RNA cleavage activity (Yuan et al., 1992; Yuan and Altman, 1994), hairpin ribozyme structures (Berzal-Herranz et al., 1992; Chowrira et al., 1993) and hepatitis ≢ virus based ribozymes (Perrotta and Been, 1992). The general design and optimization of ribozyme directed RNA cleavage activity has been discussed in detail (Haseloff and Gerlach, 1988; Symons, 1992; Chowrira, et al., 1994; and Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (A,C or U; Perriman, et al., 1992; Thompson, et al., 1995). The frequency of this dinucleotide occurring in any given RNA is statistically 3 out of 16. Therefore, for a given target messenger RNA of 1000 bases, 187 dinucleotide cleavage sites are statistically possible. The message for IGFBP-2 targeted here are greater than 1400 bases long, with greater than 260 possible cleavage sites.

Designing and testing ribozymes for efficient cleavage of a target RNA is a process well known to those skilled in the art. Examples of scientific methods for designing and testing ribozymes are described by Chowrira et al. (1994) and Lieber and Strauss (1995), each incorporated by reference. The identification of operative and preferred sequences for use in dehydrogenase-targeted ribozymes is simply a matter of preparing and testing a given sequence, and is a routinely practiced “screening” method known to those of skill in the art.

G. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

H. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

I. Nucleic Acid Segments

In certain embodiments, the nucleic acid is a nucleic acid segment. As used herein, the term “nucleic acid segment,” are smaller fragments of a nucleic acid, such as for non-limiting example, those that encode only part of the dehydrogenase peptide or polypeptide sequence. Thus, a “nucleic acid segment” may comprise any part of a gene sequence, of from about 2 nucleotides to the full length of the dehydrogenase peptide or polypeptide encoding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

In a non-limiting example, nucleic acid segments may contain up to 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, or 5000 nucleotides. Contiguous nucleic acids segments of SEQ ID NO: 1, 3, 5, 7, 9, 11, 13, 15, 17, 19, 21, 23, 24 and 25 may be used in the present invention. Nucleic acid segments may also contain up to 10,000, 20,000, 30,000, 50,000, 100,000, 250,000, 500,000, 750,000, to 1,000,000 nucleotides in length, as well as constructs of greater size, up to and including chromosomal sizes are contemplated for use in the present invention.

J. Nucleic Acid Complements

The present invention also encompasses a nucleic acid that is complementary to the nucleic acid encoding for a dehydrogenase. In particular embodiments the invention encompasses a nucleic acid or a nucleic acid segment complementary to the sequence set forth in SEQ ID NO: 1, 3, 5, 7, 9, 11, 13, 15, 17, 19, 21, 23, 24 and 25. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

K. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

As used herein “wild-type” refers to the naturally occurring sequence of a nucleic acid at a genetic locus in the genome of an organism, or a sequence transcribed or translated from such a nucleic acid. Thus, the term “wild-type” also may refer to an amino acid sequence encoded by a nucleic acid. As a genetic locus may have more than one sequence or alleles in a population of individuals, the term “wild-type” encompasses all such naturally occurring allele(s). As used herein the term “polymorphic” means that variation exists (i.e., two or more alleles exist) at a genetic locus in the individuals of a population. As used herein “mutant” refers to a change in the sequence of a nucleic acid or its encoded protein, polypeptide or peptide that is the result of the hand of man.

The present invention also concerns the isolation or creation of a recombinant construct or a recombinant host cell through the application of recombinant nucleic acid technology known to those of skill in the art or as described herein. A recombinant construct or host cell may express a dehydrogenase protein, peptide or peptide, or at least one biologically functional equivalent thereof. The recombinant host cell may be a prokaryotic cell. In a more preferred embodiment, the recombinant host cell is a eukaryotic cell. As used herein, the term “engineered” or “recombinant” cell is intended to refer to a cell into which a recombinant gene, such as a gene encoding a dehydrogenase, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene (i.e., they will not contain introns), a copy of a genomic gene, or will include genes positioned adjacent to a promoter not naturally associated with the particular introduced gene.

Herein certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a nucleic acid engineered or altered by the hand of man, and generally comprises one or more nucleic acid sequences organized by the hand of man.

In a non-limiting example, one or more nucleic acid constructs may be prepared containing about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc,; about 50,001, about 50,002, etc; about 750,001, about 750,002, etc.; about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

The term “functionally equivalent codon” is used herein to refer to codons that encode the same amino acid, such as the six codons for arginine and serine, and also refers to codons that encode biologically equivalent amino acids. For optimization of expression of genes in human cells, the codons are shown in Table 2 in preference of use from left to right. Thus, the most preferred codon for alanine is thus “GCC”, and the least is “GCG” (see Table 2, below). Codon usage for various organisms and organelles can be found at the website http://www.kazusa.or.jp/codon/, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

TABLE 2
Preferred Human DNA Codons

| Amino Acids | | | Codons |
|---|---|---|---|
| Alanine | Ala | A | GCC GCT GCA GCG |
| Cysteine | Cys | C | TGC TGT |
| Aspartic acid | Asp | D | GAC GAT |
| Glutamic acid | Glu | B | GAG GAA |
| Phenylalanine | Phe | F | TTC TTT |
| Glycine | Gly | G | GGC GGG GGA GGT |
| Histidine | His | H | CAC CAT |
| Isoleucine | Ile | I | ATC ATT ATA |
| Lysine | Lys | K | AAG AAA |
| Leucine | Leu | L | CTG CTC TTG CTT CTA TTA |
| Methionine Met | M | ATG | |
| Asparagine | Asn | N | AAC AAT |
| Proline | Pro | P | CCC CCT CCA CCG |
| Glutamine | Gln | Q | CAG CAA |
| Arginine | Arg | R | CGC AGG CGG AGA CGA CGT |
| Serine | Ser | S | AGC TCC TCT AGT TCA TCG |
| Threonine | Thr | T | ACC ACA ACT ACG |
| Valine | Val | V | GTG GTC GTT GTA |
| Tryptophan | Trp | W | TGG |
| Tyrosine | Tyr | Y | TAC TAT |

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

The nucleic acids of the present invention encompass biologically functional equivalent dehydrogenase proteins, polypeptides, or peptides or lipofuscin proteins, polypeptides or polypeptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine dehydrogenase protein, polypeptide or peptide activity at the molecular level.

Fusion proteins, polypeptides or peptides may be prepared, e.g., where the coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that may be purified by affinity chromatography or the enzyme labeling of coding regions, respectively.

Encompassed by the invention are nucleic acid sequences encoding relatively small peptides or fusion peptides, such as, for example, peptides of from about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, to about 100 amino acids in length, or more preferably, of from about 15 to about 30 amino acids in length.

As used herein an “organism” may be a prokaryote, eukaryote, virus and the like. As used herein the term “sequence” encompasses both the terms “nucleic acid” and “proteancecous” or “proteanaceous composition.” As used herein, the term “proteinaceous composition” encompasses the terms “protein”, “polypeptide” and “peptide.” As used herein “artificial sequence” refers to a sequence of a nucleic acid not derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e., an “enzymatically produced” sequence) or biological production in vivo (i.e., a “biologically produced” sequence).

VII. Nucleic Acid Detection

In addition to their use in directing the expression of SEQ ID NO: 2, SEQ ID NO: 4 or other dehydrogenases, inhibitors of dehydrogenase, proteins, polypeptides and/or peptides, the nucleic acid sequences disclosed herein have a variety of other uses. For example, they have utility as probes or primers for embodiments involving nucleic acid hybridization. They also can be used for determining the activity of the dehydrogenase of the invention. For example, the transcript levels of 11cRD can be measured to determine the effectiveness of a therapeutic agent in reducing the activity of 11cRD.

A. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCRTM, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

B. Amplification of Nucleic Acids

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to SEQ ID NO: 1 or SEQ ID NO: 3 are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-((α-thio)-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

C. Detection of Nucleic Acids

Following any amplification, it may be desirable to separate the amplification product from the template and/or the excess primer. The detection of a transcript of a dehydrogenase can be used to determine if a candidate substance inhibits dehydrogenase activity. The detection of a nucleic acids can also be used to determine expression within a cell.

In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

D. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

E. Kits

All the essential materials and/or reagents required for detecting SEQ ID NO. 2, SEQ ID NO. 4 in a sample may be assembled together in a kit. This generally will comprise a probe or primers designed to hybridize specifically to individual nucleic acids of interest in the practice of the present invention, including SEQ ID NO. 1, SEQ ID NO. 3. Also included may be enzymes suitable for amplifying nucleic acids, including various polymerases (reverse transcriptase, Taq, etc.), deoxynucleotides and buffers to provide the necessary reaction mixture for amplification. Such kits may also include enzymes and other reagents suitable for detection of specific nucleic acids or amplification products. Such kits generally will comprise, in suitable means, distinct containers for each individual reagent or enzyme as well as for each probe or primer pair.

VIII. Nucleic Acid Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operable linked coding sequence in a particular host cell. In addition to control sequences that govern transcription and translation, vectors and expression vectors may contain nucleic acid sequences that serve other functions as well and are described infra.

A. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 3 lists non-limiting examples of elements/promoters that may be employed, in the context of the present invention, to regulate the expression of a RNA. Table 4 provides non-limiting examples of inducible elements, which are regions of a nucleic acid sequence that can be activated in response to a specific stimulus.

TABLE 3
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton et |
| | al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; Goodbourn et |
| | al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek et |
| | al., 1987; Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., |
| | 1983; de Villiers et al., 1984; Hen et al., 1986; Satake et |
| | al., 1988; Campbell and/or Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Choi et al., 1988; Reisman et al., |
| | 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., |
| | 1987; Stephens et al., 1987 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits et |
| | al., 1988; Feng et al., 1988; Takebe et al., 1988; Rosen et |
| | al., 1988; Berkhout et al., 1989; Laspia et al., 1989; Sharp |
| | et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 4
Inducible Elements

| Element | Inducer | References |
|---|---|---|
| MT II | Phorbol Ester (TFA) | Palmiter et al., 1982; Haslinger et |
| | Heavy metals | al., 1985; Searle et al., 1985; |
| | | Stuart et al., 1985; Imagawa et |
| | | al., 1987, Karin et al., 1987; |
| | | Angel et al., 1987b; McNeall et |
| | | al., 1989 |
| MMTV (mouse mammary | Glucocorticoids | Huang et al., 1981; Lee et al., |
| tumor virus) | | 1981; Majors et al., 1983; |
| | | Chandler et al., 1983; Lee et al., |
| | | 1984; Ponta et al., 1985; Sakai et |
| | | al., 1988 |
| β-Interferon | Poly(rI)x | Tavernier et al., 1983 |
| | Poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP7O | E1A, SV4O Large T | Taylor et al., 1989, 1990a, 1990b |
| | Antigen | |
| Proliferin | Phorbol Ester-TPA | Mordacq et al., 1989 |
| Tumor Necrosis Factor α | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Nonlimiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

B. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picomavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

C. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

D. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

E. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

F. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

G. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

H. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

I. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, a rod cell.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

J. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Vector components of the present invention may be a viral vector that encode one or more candidate substance or other components such as, for example, an immunomodulator or adjuvant for the candidate substance. Non-limiting examples of virus vectors that may be used to deliver a nucleic acid of the present invention are described below.

1 . Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the candidate substances of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McL aughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as an antigen delivery vectors in vaccines of the candidate substances due to their ability to integrate their genes into the host genome, transferring a large amount of foreign genetic material, infecting a broad spectrum of species and cell types and of being packaged in special cell-lines (Miller, 1992).

In order to construct a vaccine retroviral vector, a nucleic acid (e.g., one encoding an dehydrogenase or therapeutic agent antigen of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Couparet al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

K. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

L. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

In certain embodiments, the host cell or tissue may be comprised in at least one organism. In certain embodiments, the organism may be, but is not limited to, a prokayote (e.g., a eubacteria, an archaea) or an eukaryote, as would be understood by one of ordinary skill in the art (see, for example, webpage http://phylogeny.arizona.-edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

It is an aspect of the present invention that the nucleic acid compositions described herein may be used in conjunction with a host cell. For example, a host cell may be transfected using SEQ ID NO: 3.

M. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'S COMPLETE CONTROL™ Inducible Mammalian Expression System, which involves a synthetic ecdysone-inducible receptor, or its pET Expression System, an E. Coli expression system. Another example of an inducible expression system is available from INVITROGEN®, which carries the T-RES™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g. 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.-nlm.nih.gov/). The coding regions for these known genes may be amplified and/or expressed using the techniques disclosed herein or by any technique that would be know to those of ordinary skill in the art. Additionally, peptide sequences may be synthesized by methods known to those of ordinary skill in the art, such as peptide synthesis using automated peptide synthesis machines, such as those available from Applied Biosystems (Foster City, Calif.).

IX. Proteinaceous Compositions

In certain embodiments, the present invention concerns novel compositions comprising at least one proteinaceous molecule. The proteinaceous molecule may be used as a candidate substance to be screened as an inhibitor of a dehydrogenase. The proteinaceous molecule may also be used, for example, in a pharmaceutical composition for the delivery of a therapeutic agent or as part of a screening assay in the determination of dehydrogenase activity. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, but is not limited to, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino molecule residues, and any range derivable therein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid, including but not limited to those shown on Table 5 below.

TABLE 5
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, | AHyl | allo-Hydroxylysine |
| | β-Amino-propionic acid | | |
| Abu | 2-Aminobutyric acid | 3Hyp | 3-Hydroxyproline |
| 4Abu | 4-Aminobutyric acid, piperidinic | 4Hyp | 4-Hydroxyproline |
| | acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Organisms include, but are not limited to, Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nhn.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may comprise at least one antibody. It is contemplated that antibodies to specific tissues may bind the tissue(s) and foster tighter adhesion of the glue to the tissues after welding. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

Proteins and peptides suitable for use in this invention may be autologous proteins or peptides, although the invention is clearly not limited to the use of such autologous proteins. As used herein, the term “autologous protein, polypeptide or peptide” refers to a protein, polypeptide or peptide which is derived or obtained from an organism. Organisms that may be used include, but are not limited to, a bovine, a reptilian, an amphibian, a piscine, a rodent, an avian, a canine, a feline, a fungal, a plant, or a prokaryotic organism, with a selected animal or human subject being preferred. The “autologous protein, polypeptide or peptide” may then be used as a component of a composition intended for application to the selected animal or human subject. In certain aspects, the autologous proteins or peptides are prepared, for example from whole plasma of the selected donor. The plasma is placed in tubes and placed in a freezer at about −80° C. for at least about 12 hours and then centrifuged at about 12,000 times g for about 15 minutes to obtain the precipitate. The precipitate, such as fibrinogen may be stored for up to about one year (Oz, 1990).

X. Protein Purification

To prepare a composition comprising the candidate substance or dehydrogenase, it may be desirable to purify the components or variants thereof. According to one embodiment of the present invention, purification of a peptide comprising the candidate substance or dehydrogenase can be utilized ultimately to operatively link this domain with a selective agent. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide, such as a dehydrogenase. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition, such as the dehydrogenase, that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

A. Synthetic Peptides

The present invention also describes a dehydrogenase, including an fusion protein, for use in various embodiments of the present invention. The peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Peptides with at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 55, 60, 65, 70, 75, 80, 85, 90, 95 or up to about 100 amino acid residues are contemplated by the present invention.

The compositions of the invention may include a peptide comprising a dehydrogenase that has been modified to enhance its activity or to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity.

Compositions for use in the present invention may also comprise peptides that include all L-amino acids, all D-amino acids, or a mixture thereof. The use of D-amino acids may confer additional resistance to proteases naturally found within the human body and are less immunogenic and can therefore be expected to have longer biological half lives.

XI. Screening for Modulators of the Protein Function

The present invention further comprises methods for identifying modulators of a dehydrogenase such as 11cRD. Modulation of a dehydrogenase involves altering or changing it; transcription, translation expression (transcription+translation), post-translational modification, processing, turnover rate, location or translocation, secretion, activity and/or function of a dehydrogenase may be altered. The preferred modulation is a modulation of the dehydrogenase activity. These assays may comprise random screening of large libraries of candidate substances; alternatively, the assays may be used to focus on particular classes of compounds selected with an eye towards structural attributes that are believed to make them more likely to modulate the function of the dehydrogenase.

By function, it is meant that one may assay for a measurable effect on a candidate substance activity or dehydrogenase inhibition by the candidate substance. To identify a dehydrogenase modulator, one generally will determine the activity or level of inhibition of dehydrogenase in the presence and absence of the candidate substance, wherein a modulator is defined as any substance that alters these characteristics. For example, a method generally comprises: (a) providing a candidate modulator; (b) admixing the candidate modulator with an isolated compound or cell, or a suitable experimental animal; (c) measuring one or more characteristics of the compound, cell or animal in step (b); and (d) comparing the characteristic measured in step (c) with the characteristic of the compound, cell or animal in the absence of said candidate modulator, wherein a difference between the measured characteristics indicates that said candidate modulator is, indeed, a modulator of the compound, cell or animal.

Assays may be conducted in cell free systems, in isolated cells, or in organisms including transgenic animals. It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may potentially modify dehydrogenase function. The candidate substance may inhibit or enhance dehydrogenase activity or alter sensitivity to dehydrogenase inhibition. The candidate substance may be a protein or fragment thereof, a small molecule, or even a nucleic acid molecule. An example of pharmacological compounds will be compounds that are structurally related to dehydrogenase, or a substrate of a dehydrogenase. Using lead compounds to help develop improved compounds is know as “rational drug design” and includes not only comparisons with known inhibitors and activators, but predictions relating to the structure of target molecules. An “inhibitor” is a molecule which represses or prevents another molecule from engaging in a reaction. An “activator” is a molecule that increases the activity of an enzyme or a protein that increases the production of a gene product in DNA transcription.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are well known to those of skill in the art. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on dehydrogenase. Regardless of the type of inhibitor or activator identified by the present screening methods, the effect of the inhibition or activator by such a compound results in alteration in dehydrogenase enzymatic activity or susceptibility to dehydrogenase inhibition as compared to that observed in the absence of the added candidate substance.

B. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule such as a dehydrogenase or 11-cis-retinal dehydrogenase in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

C. In cyto Assays

The present invention also contemplates the screening of compounds for their ability to modulate the activity or other properties of a dehydrogenase such as 11cRD in cells. Various cell lines can be utilized for such screening assays, including cells specifically engineered for this purpose.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

D. In vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In the current invention, the preferred animal model for screening for modulation in the function of dehydrogenases such as 11-cis-retinol dehydrogenase is knockout mouse model containing a null mutation in the abcr gene for RmP.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

The present invention provides methods of screening for a candidate substance that changes the activity of a dehydrogenase such as 11-cis-retinol. In these embodiments, the present invention is directed to a method for determining the ability of a candidate substance to changes the activity of a dehydrogenase, generally including the steps of: administering a candidate substance to the animal; and determining the ability of the candidate substance to reduce one or more characteristics of a dehydrogenase.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

XII. Immunological Reagents

In certain aspects of the invention, one or more antibodies against a dehydrogenase may be produced. These antibodies may be used in various diagnostic or therapeutic applications, described herein below. An antibody can be used as a candidate substance in the screening assay to determine if the antibody an inhibitor of a dehydrogenase such as 11cRD. An antibody to a dehydrogenase may also be used to measure the concentration of the dehydrogenase.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

A wide range of animal species can be used for the production of antisera. Typically the animal used for production of antisera is a rabbit, a mouse, a rat, a hamster, a guinea pig or a goat. The choice of animal may be decided upon the ease of manipulation, costs or the desired amount of sera, as would be known to one of skill in the art.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, chemokines, cofactors, toxins, plasmodia op synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, NJ), cytokines such as γ-interferon, IL-2, or IL-12 or genes encoding proteins involved in immune helper functions, such as B-7.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

It is also contemplated that a molecular cloning approach may be used to generate monoclonals. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10 times as many antibodies can be produced and screened in a single round, and that new specificities are generated by H and L chain combination which further increases the chance of finding appropriate antibodies. In another example, LEEs or CEEs can be used to produce antigens in vitro with a cell free system. These can be used as targets for scanning single chain antibody libraries. This would enable many different antibodies to be identified very quickly without the use of animals.

Alternatively, monoclonal antibody fragments encompassed by the present invention can be synthesized using an automated peptide synthesizer, or by expression of full-length gene or of gene fragments in E. coli.

A. Antibody Conjugates

The present invention further provides antibodies to dehydrogenase transcribed messages and translated proteins, polypeptides and peptides, generally of the monoclonal type, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kanget al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

B. Immunodetection Methods

In still further embodiments, the present invention concerns immunodetection methods for binding, purifying, removing, quantifying and/or otherwise generally detecting biological components such as dehydrogenase-expressed message(s), protein(s), polypeptide(s) or peptide(s). Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of containing dehydrogenase expressed message and/or protein, polypeptide and/or peptide, and contacting the sample with a first anti-dehydrogenase message and/or anti-dehydrogenase translated product antibody in accordance with the present invention, as the case may be, under conditions effective to allow the formation of immunocomplexes.

These methods include methods for purifying an dehydrogenase message, protein, polypeptide and/or peptide from organelle, cell, tissue or organism's samples. In these instances, the antibody removes the antigenic dehydrogenase message, protein, polypeptide and/or peptide component from a sample. The antibody will preferably be linked to a solid support, such as in the form of a column matrix, and the sample suspected of containing the dehydrogenase message, protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen, and contact the sample with an antibody against the dehydrogenase produced antigen, and then detect and quantify the amount of immune complexes formed under the specific conditions.

In terms of antigen detection, the biological sample analyzed may be any sample that is suspected of containing an antigen, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum, although tissue samples or extracts are preferred.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any dehydrogenase antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The dehydrogenase antigen antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the diagnosis and prognosis of conditions such as various diseases wherein a specific dehydrogenase is expressed, such as an viral dehydrogenase of a viral infected cell, tissue or organism; a cancer specific gene product, etc. Here, a biological and/or clinical sample suspected of containing a specific disease associated dehydrogenase expression product is used. However, these embodiments also have applications to non-clinical samples, such as in the titering of antigen or antibody samples, for example in the selection of hybridomas.

In the clinical diagnosis and/or monitoring of patients with various forms a disease, such as, for example, macular or retinal degeneration, the detection of a macular specific gene product, and/or an alteration in the levels of a macular or retinal degeneration specific gene product, in comparison to the levels in a corresponding biological sample from a normal subject is indicative of a patient with macular or retinal degeneration. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive. Of course, the antibodies of the present invention in any immunodetection or therapy known to one of ordinary skill in the art.

1. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, the anti-dehydrogenase message and/or anti-dehydrogenase translated product antibodies of the invention are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another anti-dehydrogenase message and/or anti-dehydrogenase translated product antibody that is linked to a detectable label. This type of ELISA is a simple “sandwich ELISA”. Detection may also be achieved by the addition of a second anti-dehydrogenase message and/or anti-dehydrogenase translated product antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with the anti-dehydrogenase message and/or anti-dehydrogenase translated product antibodies of the invention. After binding and/or washing to remove non-specifically bound immune complexes, the bound anti-dehydrogenase message and/or anti-dehydrogenase translated product antibodies are detected. Where the initial anti-dehydrogenase message and/or anti-dehydrogenase translated product antibodies are linked to a detectable label, the immune complexes may be detected directly. Again, the immune complexes may be detected using a second antibody that has binding affinity for the first anti-dehydrogenase message and/or anti-dehydrogenase translated product antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

2. Inmunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

XIII. Pharmaceutical Preparations

Pharmaceutical compositions of the present invention comprise an effective amount of one or more candidate substance, therapeutic agents, a dehydrogenase or additional agent dissolved or dispersed in a pharmaceutically acceptable carrier. It is an aspect of the invention that candidate substance, which is shown to modulate a dehydrogenase such as 11-cis retinol dehydrogenase or effect the amount of lipofuscin or a component of lipofuscin in a cell, be prepared for pharmaceutical administration. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one dehydrogenase, candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The dehydrogenase or candidate substance may comprise different types of carriers depending on whether it is to be administered in solid, liquid or aerosol form, and whether it need to be sterile for such routes of administration as injection. The present invention can be administered intraocularly, intravenously, intradermally, intraarterially, intraperitoneally, intracranially, topically, intramuscularly, intraperitoneally, subcutaneously, intravesicularlly, mucosally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

certain embodiments, pharmaceutical compositions may comprise, for example, at least about 0. 1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The dehydrogenase or candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, the dehydrogenase or candidate substance is prepared for administration by eye drops. The pupil may be dilated prior to administration of the candidate substance.

In certain embodiments the dehydrogenase or candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments, the composition may comprise an ophthalmic solution, an ophthalmic suspension, an ophthalmic ointment, an ocular insert or an intraocular solution. Other modes of administration to the eye include packs, intracameral injections which are made directly into the anterior chamber, iontophoresis wherein an eyecup bearing an electrode keeps the therapeutic agent in contact with the cornea, subconjunctival injections for the introduction of therapeutic agents that do not penetrate into the anterior segment or penetrate too slowly and retrobulbar injections for delivery of therapeutic agents predominantly into the posterior section of the globe.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

XIV. Combinational Therapies

Therapies for macular degeneration, known to one of skill in the art, may be used in combination with the therapeutic agent obtained from screening using dehydrogenases of the present invention. Thus, in order to increase the effectiveness of the therapy using a therapeutic agent, or expression construct coding therefor, it may be desirable to combine these compositions with other agents effective in the treatment of macular degenerations such as but not limited to those described below. For example, one can use one can use the therapeutic agent-based therapy in conjunction with surgery and/or supplements, and/or radiation, an/or other therapeutic methods.

The other therapy may precede or follow the therapeutic agent-based therapy by intervals ranging from minutes to days to weeks. In embodiments where the other macular or retinal degeneration therapy and the therapeutic agent-based therapy are administered together, one would generally ensure that a significant period of time did not expire between the time of each delivery. In such instances, it is contemplated that one would administer to a patient both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the other macular or retinal degeneration therapy and the therapeutic agent-based therapy will be required to prevent blindness or an decrease in vision. Various combinations may be employed, where the other macular or retinal degeneration therapy is “A” and the therapeutic agent-based therapy treatment is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations also are contemplated. The exact dosages and regimens can be suitable altered by those of ordinary skill in the art.

Treatments developed that reduce the risk of vision loss in selected patients with “wet” macular degeneration include photocoagulation and photodynamic therapy. These therapies may be used in conjunction with a therapeutic agent which has been through screening using a dehydrogenase.

Laser photocoagulation involves laser surgery in the early stages of the disease. Thermal energy is delivered under topical anaesthesia to the retina to burn the area containing choroidal neovascularisation. Currently, this immediate surgery is often believed to be necessary if vision is to be saved. However, laser surgery may lead to scarring of the macula and additional vision loss. (www.macular.org/wet.html) Several clinical trials (Macular Photocoagulation Study Groups, 1991(a), 1991(b), 1993) have shown that photocoagulation reduces the risk of severe vision loss for about 15% of patients. (Bressler et al., 1987; Moisseiev et al., 1995) The photocoagulation treatment is usually applicable to choroidal neovascular lesions that do not extend under the center of the retina since photocoagulation will usually destroy any viable photoreceptors overlying the abnormal vessels (Bressler et al., 2000).

Photodynamic therapy, allows for the treatment of patients with neovascular macular degeneration having vessels extending under the center of the retina. Photodynamic therapy uses the drug verteporfin, and has recently been shown to reduce the risk of moderate and severe vision loss (Treatment of Age-Related Macular Degeneration with Photodynamic Therapy (TAP) Study Group, 1999, 2000) In photodynamic therapy, a photoactivator, verteporfin, is injected into a patients vein where it then travels to the eye and becomes concentrated within the neovascular lesion. Then a laser is applied over the entire neovascular lesion to activate the drug. The photoactivated verteporfin selectively destroys lesions by creating reactive intermediates of oxygen such as superoxide and hydroxide radicals without damaging viable retinal tissue overlying the neovascularisation (Hasan et al., 2000). Retreatment as often as every three months are needed to prevent significant growth. The laser used in photodynamic therapy is not a “heat producing” laser as used in photocoagulation. Generally, this therapy works for blood vessels that are not covered by blood, fat, or fluid in growths wherein the neovascularization is less than about 50% (www.macular-degeneration.org/porphyrin/porphyrin.html). Clinical trials have shown that photo-dynamic therapy with verteporfin could reduce the risk of moderate and severe vision loss from 61% to 33% at one year and from 69% to 41% at two years in patients with neovascularisation extending under the center of the retina and predominantly classic appearances on fluorescein angiography (Hasan et al., 2000).

Radiation therapy for “wet” macular degeneration is used to destroy blood vessels and prevent neovascularization. Radiation therapy is useful after surgery to prevent or reduce scarring by killing or effecting the cells which make up newly formed blood vessels, inflammatory cells which promote scarring and cells which help create fibrous tissues (Finger et al., 1998). Bergink et al. (1998) have demonstrated radiation can delay vision loss after the onset of “wet” macular degeneration. In a clinical trial comparing moderate doses of external beam irradiation to observation (no treatment), Bergink found that 22% of the radiation treated patients had 6 lines of vision loss as compared 44% of the untreated patients at 1 year follow up. Preferred forms of radiation for use in treatment of macular degeneration include: proton beam, strontium-90, palladium-103, radiosurgery, and EBRT (external beam radiation therapy). Potential therapeutic effects and side-effects of the different forms of radiation will vary due to the way radiation is distributed within the macula and ocular structures.

A number of new drugs have promise for the prevention or delay of photoreceptor cell death and retinal degeneration. These drugs include PKC 412 (which blocks chemicals in the body that foster new blood vessel growth, or angiogenesis), Glial Derived Neurotrophic Factor (a survival factor which has slowed degeneration in a rodent model), and diatazem (a calcium-channel blocker which addresses a rare retinal gene defect called beta PDE). (http://www.homesweetweb.com/MDPeople/Files/research.html). The use of supplemental lutein, a caratonoid, has been shown to cause short term improvements in vision retinitis pigmentosa (RP) and related retinal degenerations (Dagnelie et al., 2000). Vitamin A, vitamin B complexes, β-carotene and other supplements are also beneficial in treating patients with macular degeneration. Other techniques for the treatment of macular or retinal degeneration include gene replacement therapy, microchip implantation, photoreceptor cell transplantation, macular translocation surgery, and drusen lasering.

XV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Tissue Preparation

Mouse Tissues. New-born wild-type and abcr−/− mice, both on an inbred 129-strain background, were raised under normal 12-hr cyclic illumination (25-30 lux during light phase), or under total darkness in a ventilated cabinet for up to 18 weeks. Groups of six mice were analyzed per light condition. In a separate study, abcr−/− mice (hybrid strain 129×B6) were raised for 12-weeks under 12-hr cyclic illumination, transferred to the dark, and groups of three mice were analyzed at 12, 16, 20, and 28-weeks. Mice were anesthetized with intraperitoneal ketamine (200 mg/kg) plus xylazine (10 mg/kg) and sacrificed by cervical dislocation. In experiments involving a photobleach, the pupils of anesthetized mice were dilated with 1.0% atropine sulfate and the animals were exposed to 400 lux illumination in a Ganzfeld dome for five minutes, resulting in ˜45% bleaching of rhodopsin (Weng et al., 1999). Retinas were dissected from the posterior poles of each eye. The remaining RPE/eyecups were washed in PBS (pH 7.2). All dissections and tissue manipulations were performed on ice in dim red light.

Human Tissues. Sections of retina and overlying RPE were provided as postmortem specimens through the Histopathology Program of the Foundation Fighting Blindness (FFB). The FFM specimens were from a 62-year-old female with a clinical history of FFM (FFB #601), confirmed by published histopathological analysis (Birnbach et al., 1994). The postmortem interval (time of death to fixation) was 4.5 hours. The STGD1 specimens were from a 73-year-old male with a clinical history of STGD1 (FFB #219). The postmortem interval was ˜12 hours. Seven normal human retina and RPE specimens from patients with no retinal pathology were provided by Ann Milam, University of Pennsylvania. Ages ranged from 71 to 85 yrs (mean=77). Postmortem intervals ranged from 3 to 16 hrs (mean=6.9). All tissues were fixed in 4% paraformaldehyde plus 0.5% glutaraldehyde and stored in 2% paraformaldehyde. For each specimen, 0.5×0.5 cm sections from the perimacular region of the retina and overlying RPE were analyzed after rinsing in Ringer's solution (pH 7.4) to remove fixative.

Tissue Preparation and Extraction. Mouse and human tissues were homogenized in 1 ml of PBS. For analysis of mouse outer segments, dissected retinas were shaken on a vortex mixer in 8% Optiprep/Ringer's buffer and fractionated by centrifugation through an Optiprep step-gradient according to the procedure of Tsang et al. (1998). Collected outer segments were suspended in 1 ml PBS and homogenized. 1 ml of chloroform/methanol (2:1, v/v) was added to each homogenate. Phospholipids and chloroform-soluble fluorophores were extracted twice from the samples following addition of 4 ml of chloroform and 3 ml water. The pooled organic phases were dried under a stream of argon. Sample residues were resuspended in 200- 400 μl of hexane, and analyzed by HPLC.

### EXAMPLE 2

### Analysis of Phospholipids, Lipofuscin, and Retinoids

Sample extracts were analyzed by normal phase HPLC using an HP 1100 liquid chromatograph with a photodiode array detector. Chromatography conditions were as previously described (Birnbach et al., 1994) using a silica column (Microsorb 5 μm Si, 250×4.6 mm) and the mobile phase: hexane/2-propanol/ethanol/25 mM potassium phosphate/acetic acid (485:376:100:40:0.275, v/v; pH 7.0). For A2PE-H₂ purification, the water content was reduced to 2.5% for better resolution of fatty acyl esters. Spectra for the eluted peaks were corrected by subtracting the nearest integrated baseline to remove solvent absorption. A2-E, phosphatidylethanolamine, and all-trans-RAL were quantitated by comparing the sample peak-area to a calibration curve constructed with authentic standards (18:0-22:6 for the phosphatidylethanolamine standard) (Birnbach et al., 1994). The yield of all-trans-RAL was lower than previously observed for total retina (Birnbach et al., 1994) due to losses incurred during the preparation of outer segments. In Table 6, APE and protonated-APE were quantitated using published molar extinction coefficients (Anderson et al., 1996).

Fatty Acid Analysis. Purified phospholipids were treated with 2% KOH and incubated at 100° C. for 2 min (DeMar et al., 1996). The mixtures were diluted in 1 ml water, acidified with concentrated HCL (10 μl/ml sample), and the free fatty acids (FFA) were partitioned into hexane (4 ml). The hexane extract was evaporated to dryness under argon gas, and the sample residue dissolved in 100 μl acetonitrile. FFAs were resolved by reverse phase HPLC (Zorbax Eclipse XDB-C8, 5 μm, 4.6×150 mm) in acetonitrile/water/acetic acid (90:10:0.5, v/v) at 1 ml/min with a detection wavelength of 210 nm. Recovery was >90% as determined by equimolar recovery of 22:6 and 18:0 FFAs following saponification of authentic 22:6-18:0 phosphatidylethanolamine.

Mass Spectrometry. Fast-atom bombardment (FAB) mass spectrometry was performed in a glycerol matrix using a Micromass Quattro II triple-quadrapole mass spectrometer on samples purified by HPLC. Samples were dissolved in methanol/chloroform (2:1)+1% acetic acid. Samples were continuously introduced into the source at a rate of 5 μl/min with an infusion pump. Masses were established in positive-ion mode. The FAB data were confirmed by electrospray mass spectrometry (ESI) using the same instrument equipped with the manufacturer's standard electrospray source. FAB and ESI spectra were acquired every 5 seconds over an m/z range of 50-1050.

### EXAMPLE 3

### Conversion of A2PE-H₂ to A2-E in vitro

A2PE-H₂ was extracted from outer segments of abcr−/− mice and purified by HPLC as described above (2.5% water content, flow=0.75 ml/min). The purified sample in phospholipid mobile-phase was evaporated to dryness under a stream of argon and dissolved in 100 μl of phospholipid mobile-phase in equilibrium with room air. 1 μl of cold 10 N HCl was added (final concentration 100 mN). Aliquots were incubated for 5 min at 25° C. or for longer periods at −20° C., and analyzed by normal phase HPLC. To test the role of an oxidizing environment on conversion of A2PE-H₂ to A2PE and A2E, purified A2PE-H₂ from 11-month abcr−/− retinas was suspended in phospholipid mobile-phase. One aliquot was treated with 100 mN HCl and another with 100 mN HCl plus 2 mM dithiothreitol. After incubation as above, samples were analyzed by normal phase HPLC.

### EXAMPLE 4

### Increased APE in abcr−/− Outer Segments After Light Exposure

HPLC analysis was done on outer-segment extracts from wild-type and abcr−/− mice after a 45% photobleach. Although the levels of all-trans-RAL were similar between wild-type and abcr−/− outer segments, a significantly greater fraction of the all-trans-RAL in abcr−/− was present as N-retinylidene-phosphatidylethanolamine (APE) (Anderson et al., 1996; Poincelo et al., 1998) (Table 7). The ratio of protonated to unprotonated APE was also different in wild-type and abcr−/− outer segments (Table 7). Similar ratios of protonated to unprotonated APE were observed when tissue samples were homogenized directly in chloroform/methanol, suggesting that the extraction buffer had no effect on the protonation state of APE.

TABLE 6
Phosphatidylethanolamine, All-trans-RAL, and APE in Outer Segments

| Geno- | | All-trans-RAL, | APE, | [H+] APE, | % all-trans- |
|---|---|---|---|---|---|
| type | EP, pmol/eye | pmol/eye | pmol/eye | pmol/eye | RAL as APE |
| Wild Type | 17,200 ± 1,930 | 72.2 ± 1.9 | 13.8 ± 1.4 | 3.48 ± 1.31 | 24% |
| abcr-/- | 33,300 ± 4,680 | 74.1 ± 2.9 | 40.8 ± 3.6 | 4.43 ± 0.59 | 61% |

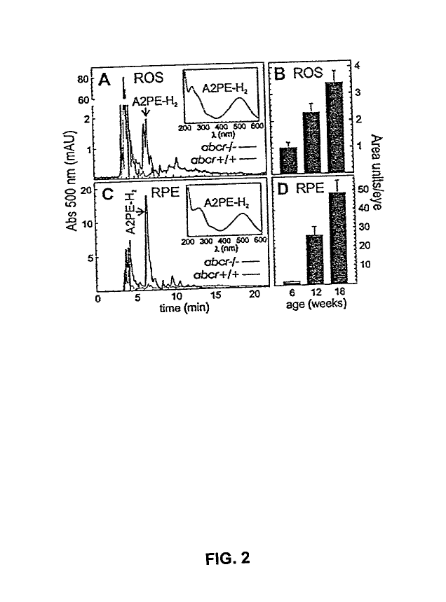

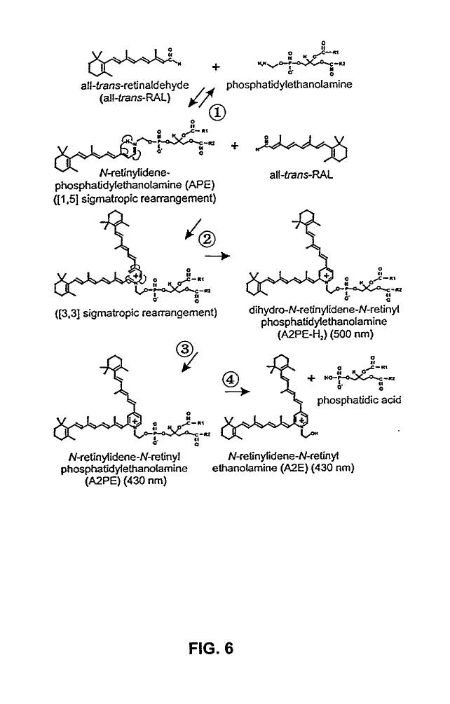

Immediately following light-exposure, APE in abcr−/− were raised significantly compared to wild-type outer-segments (Table 7). This difference is probably due to increased phosphatidylethanolamine in the mutants, since the levels of all-trans-RAL were similar in mice of both genotypes. Most of the excess APE in abcr−/− outer segments was unprotonated, possibly indicating higher intradiscal pH immediately following a photobleach. This form, in contrast to protonated APE, is capable of sigmatropic rearrangement and subsequent condensation with a second molecule of all-trans-RAL to yield A2PE-H₂ (FIG. 6). A2PE-H₂ was abundantly present in abcr−/− outer-segments and RPE, and accumulated dramatically with age (FIG. 2). Unexpectedly, A2PE-H₂ was undetectable in wild-type outer segments despite the presence, albeit at a lower level, of APE. This suggests that clearance of APE in wild-type outer segments is significantly faster than the rate of A2PE-H₂ formation.

### EXAMPLE 5

### A2PE-H₂ in Outer Segments and RPE of abcr−/− Mice

The presence of elevated APE in abcr−/− outer segments suggests that a second condensation with all-trans-RAL may occur to yield a bis-retinoid conjugate of phosphatidylethanolamine. Molecules of this type were identified by analyzing phospholipid extracts from 12-week-old wild-type and abcr−/− outer-segments and RPE. An abundant molecular species with absorption maxima (λₘₐₓ) at 205 and 500 nm was identified in abcr−/− outer-segments and RPE, but was undetectable in wild-type tissues (FIG. 2). This species was named A2PE-H₂. A2PE-H₂ was undetectable in samples of abcr−/− rest-of-retina (depleted of outer segments), suggesting that in retina, A2PE-H₂ is located exclusively in outer segments. Moreover, A2PE-H₂ accumulated dramatically with advancing age (FIGS. 1B, 1D). The presence of multiple peaks in the A2PE-H₂ fractions is due to heterogeneity of the associated fatty acyl esters, as discussed below.

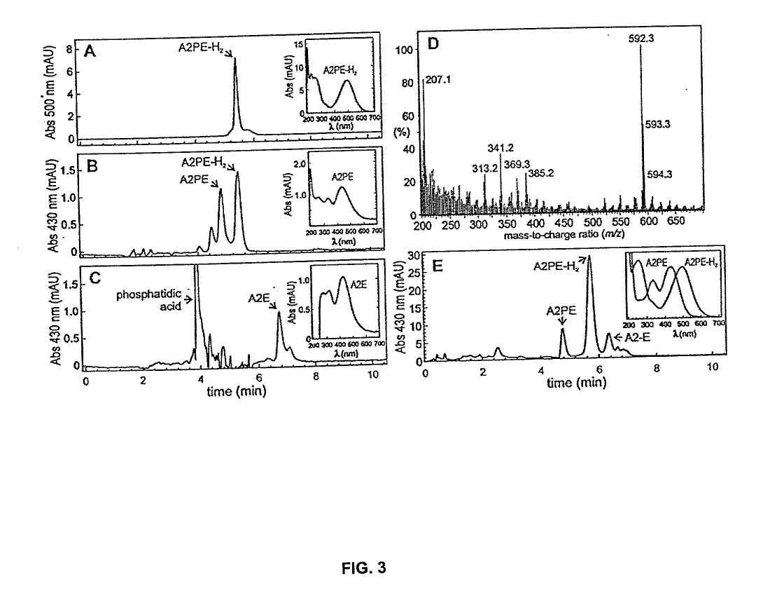

Brief incubation of A2PE-H₂ in HCl resulted in the appearance of a second chromatographic peak with different spectral properties, named A2PE (FIG. 3). Overnight incubation in HCl resulted in the complete conversion of A2PE-H₂ to A2E and phosphatidic acid. Incubation in similar medium with the addition of a mild reducing agent completely suppressed formation of A2PE and A2E, but had no effect on the release of phosphatidic acid. Thus, oxidation of the pyridinium ring and hydrolysis of the phosphate ester represent independent steps in the formation of A2E. A2PE and A2E both had a λₘₐₓ of 430 nm. This was expected, since these molecules have identical resonating structures (FIG. 6). Loss of the phospholipid moiety in A2E is evident spectrally by loss of the 205-nm absorption peak (FIGS. 2B & 2C insets). A2PE-H₂, which lacks the third double-bond of the pyridinium ring, has a visible λₘₐₓ of 500 nm. Identification of the A2E and phosphatidic-acid products following incubation in HCl was confirmed by mass spectrometry and HPLC, respectively. The major form of A2PE-H₂ had the identical fatty acid composition as the phosphatidic-acid product of its hydrolysis, and the predominant form of phosphatidylethanolamine in outer segments (Stinson et al., 1991). These data suggest that A2PE-H₂ is the source of phosphatidic acid after incubation in HCl, and that phosphatidylethanolamine from outer segments is the starting material for A2PE-H₂ formation. The stability of A2PE-H₂ in a non-oxidizing environment suggests that reaction 2 in FIG. 6 is effectively irreversible. A2PE-H₂ was present in both retina and RPE of abcr−/− mice. A2PE and A2E, however, were exclusively present in RPE. These observations suggest that the final two reactions in FIG. 6 occur only within the oxidizing and acidic environment of RPE phagolysosomes.

### EXAMPLE 6

### Conversion of A2PE-H₂ to A2E in vitro Upon Acidification

If A2PE-H₂ represents an A2E precursor, it should be converted to A2E under in vitro conditions that simulate the acidic and oxidizing environment of RPE phagolysosomes. Samples of A2PE-H₂ purified from abcr−/− outer-segments were incubated in phospholipid mobile-phase containing 100 mN HCl prior to HPLC. After five-minutes incubation, a second molecular species appeared with different chromatographic properties and a visible-light λₘₐₓ of 430 nm, similar to A2E (FIG. 3B). Incubation of A2PE-H₂ overnight in the same solution resulted in the disappearance of both A2PE-H₂ and A2PE, and the appearance of A2E plus phosphatidic acid (FIG. 3C). Quantitation of A2E and phosphatidic acid at the appropriate λₘₐₓ values yielded a molar ratio of ˜1:1 for these products. The identification of the A2E formed following acid treatment of A2PE-H₂ was confirmed by mass spectrometry. This resulted in a major molecular-ion (m/z) peak of 592.3 (FIG. 3D), in good agreement with the calculated molecular mass of 592.45 for A2E (C₄₂H₅₈ON). Identification of the phosphatidic acid was confirmed by thin layer chromatography and HPLC (not shown). With modification of the mobile phase, A2PE-H₂ could be separated into three chromatographic peaks. Fatty acid analysis on these separated peak fractions showed distinct fatty acid compositions, indicating different fatty acyl esters of A2PE-H₂. The major peak fraction of A2PE-H₂ from outer segments contained equimolar quantities of stearic (18:0) and docosahexanoic (22:6) acid, identical to the phosphatidic-acid product following acid incubation.

To test the dependence on an oxidizing environment upon the conversion of A2PE-H₂ to A2PE and A2E, 2 mM dithiothreitol was added to purified A2PE-H₂ in the phospholipid mobile-phase. The presence of dithiothreitol completely suppressed formation of both A2PE and A2E. However, similar amounts of phosphatidic acid were formed plus or minus dithiothreitol, indicating that acid-hydrolysis of the phosphate ester is not dependent on an oxidizing environment. In phospholipid mobile-phase without added HCl, A2PE-H₂ was slowly converted to A2PE over approximately one month at −20° C. Thus, oxidation of A2PE-H₂ is strongly accelerated by acid. Finally, analysis of a phospholipid extract of RPE from a six-month abcr−/− mouse was done without prior acid treatment. Both A2PE-H₂ and A2PE were present (FIG. 3E), indicating that A2PE is a naturally occurring intermediate in the formation of A2E, and not an artifact of acid treatment in vitro.

### EXAMPLE 7

### A2E and A2PE-H₂ in RPE from Humans with FFM and STGD1

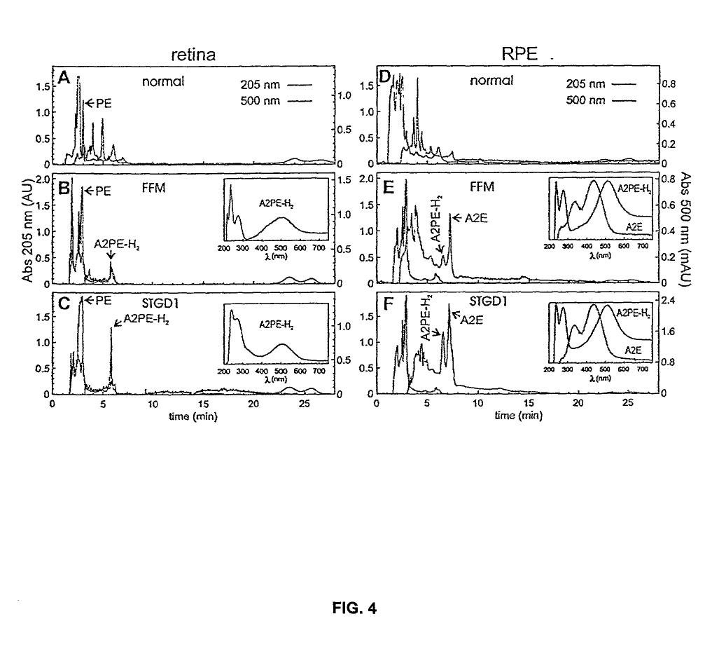

To test the validity of the abcr−/− mouse as a model for ABCR-mediated recessive macular degeneration, samples of postmortem retina and overlying RPE from the perimacular region of patients were analyzed with FFM and STGD1, and seven human controls. Representative chromatograms are shown in FIG. 4. The levels of phosphatidylethanolamine were 22 and 30 μmoles per 0.5×0.5 cm section of retina from the FFM and STGD1 patients, respectively, and 10.3±1.79 (standard deviation) μmoles per section of retina from the human controls. The levels of A2E were 33 and 61 pmoles per 0.5×0.5 cm section of RPE from the FFM and STGD1 patients, respectively, and 5.30±2.96 pmoles per section of RPE from the human controls. A2PE-H₂ was present in both retina and RPE from the patient samples, but undetectable in the controls (FIG. 4).

The biochemical abnormalities in outer segments and RPE from abcr−/− mice were similar to those observed in postmortem tissues from two patients with STGD1 and FFM. In particular, phosphatidylethanolamine was 2-3 fold more abundant in retina and A2E 6-12 fold more abundant in RPE from patients versus controls. A2PE-H₂ was abundantly present in retina and RPE from both patients, but was undetectable in control tissues (FIG. 4). The specific molecular defects in the patients presented here have not yet been defined. However, since FFM and STGD1 are both recessive diseases caused by mutations in ABCR (Allikmets et al., 1997; Rozet et al., 1998; Stone et al., 1998), these patients presumably have partial or complete loss of RmP function. The similarity of phenotypes between abcr−/− mice and humans with ABCR-mediated diseases was surprising, given that FFM and STGD1 are predominantly macular degenerations, and that mouse retina does not contain a macula. In humans, greater vulnerability of the macula to the ABCR-mediated disease process may be secondary to the higher ratio of outer segments to RPE cells in this area (Jonas et al., 1999).

### EXAMPLE 8

### A2E in the RPE of Mice Reared under Cyclic Light vs. Total Darkness

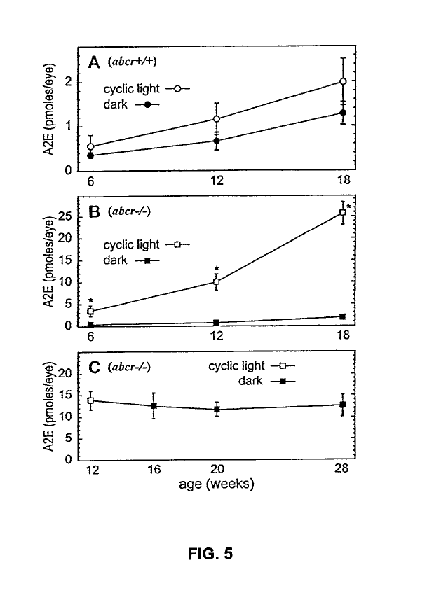

Previous observations showed significant accumulation of A2E in the RPE of abcr−/− mice (Birnbach et al., 1994). To test the dependence of this process on light-exposure history, levels of A2E in RPE from mice raised under cyclic light were compared with levels of A2E in mice raised in total darkness. At 18 weeks under cyclic light, the levels of A2E in abcr−/− mutants were dramatically higher than those of wild-type mice (25.7±2.60 vs. 1.98±0.53 pmoles/eye) (FIGS. 4A & 4B). In contrast, the levels of A2E were similar between 18-week-old wild-type and abcr−/− mice raised in total darkness (1.28±2.60 and 1.89±0.62 pmoles/eye, respectively). In a separate study, a group of abcr−/− mice were raised for 12 weeks under cyclic light and transferred to total darkness. Following transfer, no significant change in A2E levels were observed for up to 16 weeks in darkness (FIG. 5C). A2E was only present in RPE, and was undetectable in retina or isolated outer segments under all conditions. Together, these data indicate that A2E accumulation in abcr−/− mice is strongly light-dependent, and that once formed, A2E is not removed by the RPE.

One prediction of the pathway proposed in FIG. 6 is that A2E accumulation should be dependent on the availability of all-trans-RAL, and hence on light exposure. To test this hypothesis, any increase above basal levels of all-trans-RAL was blocked by raising abcr−/− mice under total darkness. The level of A2E in these animals was approximately equal to that of wild-type mice (FIG. 5A & 5B). Thus, the increased formation of A2E in abcr−/− mice is completely light dependent, which supports the proposed pathway. Interestingly, when mice raised under cyclic light for 12 weeks were transferred to the dark, no reduction in the level of A2E was observed, even after 16 weeks in total darkness (FIG. 5C). These data indicate that A2E is not cleared by the RPE in abcr−/− mutants, in contrast to the transient accumulation of lipofuscin reported in rats after intravitreal leupeptin injection (Katz et al., 1999). The observed inhibition of A2E accumulation in abcr−/− mice raised under total darkness suggests that limiting light-exposure may reduce the rate of disease progression in humans with recessive ABCR-mediated diseases. Although the mechanism of A2PE-H₂ formation undoubtedly varies with genetic etiology, the final pathway of A2E accumulation may be similar in other forms of lipofuscin-mediated macular or retinal degeneration. Pharmacologic inhibition of one or more steps in the A2E synthetic-pathway may thus be an effective treatment strategy for other macular degenerations associated with lipofuscin accumulation.

### EXAMPLE 9

### Lipofuscin Accumulation in ABCR-mediated Retinal Degeneration

Since lipofuscin accumulation in RPE cells is a common feature of STGD, AMD, and abcr−/− mice, the biochemical origin of this fluorescent pigment was sought. A major component of ocular lipofuscin is the bis-retinoid, N-retinylidene-N-retinylethanolamine (A2E) (Sakai et al., 1996; Reinboth et al., 1997). Results suggest that APE, which is elevated in abcr−/− outer-segments, condenses with a second molecule of atRAL (also elevated in abcr−/− outer-segments) to yield dihydro-N-retinylidene-N-retinyl phosphatidylethanolamine (A2PE-H₂) (Mata et al., 2000). It has long been appreciated that vertebrate photoreceptors undergo diurnal shedding of distal outer segments (Young et al., 1969). These shed membrane-packages are engulfed and digested by cells of the overlying RPE. abcr−/− outer-segments contain dramatically higher levels of A2PE-H₂ compared to wild-type controls. Brief incubation in vitro of A2PE-H₂ in an acidic and oxidizing medium resulted in the appearance of N-retinylidene-N-retinyl phosphatidylethanolamine (A2PE) (Mata et al., 2000). Overnight incubation caused complete conversion of A2PE-H₂ to A2E and phosphatidic acid. These results suggest that A2PE-H₂ is formed in photoreceptors and converted to A2E in RPE cells following phagocytosis of shed outer-segments. An additional function of RmP thus may be to prevent A2E deposition in RPE cells by eliminating A2PE-H₂ from photoreceptor outer-segments. Consistent with this model, formation of A2PE-H₂, A2PE, and A2E was strongly suppressed when abcr−/− mice were raised under total darkness (Mata et al., 2000).

The postmortem retinas and RPE from patients with STGD were analyzed for the presence of A2PE-H₂, A2PE, and A2E. Similar to abcr−/− mice, these compounds were much higher in tissues from the patients compared to age-matched human controls. PE was also elevated in STGD retinas compared to controls. Thus, virtually the complete phenotypic spectrum of STGD is reproduced in abcr−/− mice. These results establish the abcr−/− mouse as an excellent model system for STGD.

The ocular phenotype in abcr+/− heterozygotes were also examined to test for a possible correlation between AMD and ABCR mutations. A2E and its precursors, A2PE-H₂ and A2PE, were approximately four-fold more abundant in eight-month-old abcr+/− compared to wild-type retina and RPE. The levels of these substances in abcr+/− mice were about 40% the levels in abcr−/− mice. Similar to abcr−/− homozygotes, accumulation of A2PE-H₂ and A2E in abcr+/− retina and RPE was strongly dependent on light exposure. Heterozygous abcr-mutants also exhibited delayed recovery of photosensitivity and ultrastructural changes in RPE cells. These findings are consistent with the hypothesis that heterozygous mutations in ABCR predispose to the development of AMD in humans. The abcr+/− mouse appears to be a useful animal model for AMD.

A2E has been shown to inhibit lysosomal proteolysis in RPE cells (Sundelin et al., 1998; Holz et al., 1999). At high concentrations, A2E acts as a cationic detergent, dissolving cellular membranes (Eldred et al., 1993; Sparrow et al., 1999; Schutt et al., 2000). Photoreceptor degeneration in abcr−/− mice and humans with ABCR-mediated macular degeneration probably occurs in the sequence: (i) lipofuscin accumulates in cells of the RPE; (ii) RPE cells become sick and ultimately die due to A2E-mediated lysosomal toxicity; (iii) photoreceptors degenerate secondary to loss of the RPE support-role (Steinberg et al., 1985), resulting in blindness. The relative vulnerability of the macula in humans can be explained by the higher ratio of outer segments to RPE cells in this region of the retina (Jonas et al., 1992). In STGD patients, the highest concentration of lipofuscin is seen in RPE cells overlying the perifoveal region of the macula, which contains the greatest density of photoreceptors.

These results suggest a possible treatment strategy for STGD and AMD. If the level of atRAL can be reduced by therapeutic intervention, the levels of APE and A2PE-H₂ in photoreceptors should also decrease. A reduction in these A2E precursors should inhibit formation of lipofuscin in RPE cells. Since lipofuscin is toxic to RPE cells (Sundelin et al., 1998; Holz et al., 1999; Eldred et al., 1993; Sparrow et al., 1999; Schutt et al., 2000), and since an intact RPE is critical for photoreceptor viability (Steinberg et al., 1985), reducing lipofuscin accumulation should protect photoreceptors from degenerating and hence delay or prevent blindness in STGD and AMD. The first part of this effect has already been demonstrated by raising abcr−/− and abcr+/− mice under total darkness, which inhibited formation of atRAL and greatly reduced the levels of APE, A2PE-H₂, and A2E (Mata et al., 1969). Accordingly, Mata et al. (2000) suggested that limiting light exposure may slow the disease process in patients with STGD.

13-cis-retinoic acid (isotretinoin) is a potent inhibitor of 11cRDH in RPE cells (Law et al., 1989; Gamble et al., 1999). This drug is used clinically to treat acne because of unrelated effects on sebaceous glands. Night blindness is a common side effect of isotretinoin due to impaired regeneration of rhodopsin (FIG. 1A). A secondary effect of rhodopsin depletion is decreased atRAL in light-adapted retinas. A2PE-H₂ is formed in abcr−/− outer segments because of the elevated atRAL and PE (Mata et al., 2000). Reducing atRAL by treating abcr−/− mice with isotretinoin should inhibit formation of A2PE-H₂ in outer segments, and hence A2E in RPE cells. Isotretinoin may thus be an effective therapy for slowing the onset of blindness in abcr−/− mice and humans with STGD and AMD. Isotretinoin is a potential treatment for ABCR-mediated retinal degenerations.

### EXAMPLE 10

### Novel 11cRDH in Cone-dominant Retinas

The capacity of isolated cones to regenerate visual pigment, combined with the phenotype of congenital night-blindness in fundus albipunctatus, suggests that another form of 11cRDH, distinct from 11cRDH5 in RPE cells, may be present in cones. An 11cRDH catalytic-activity has been identified in membranes from cone-dominant ground-squirrel and chicken retinas. To clone the mRNA for this enzyme, the sequences of the short-chain dehydrogenases homologous were aligned to 11cRDH and used the conserved regions to design five sets of degenerate oligonucleotide-primers. PCR was done with each primer set on template cDNA from ground-squirrel and chicken retina. Preliminary database analysis of the cloned amplification products revealed two classes of mRNAs. The more prevalent class appeared to represent ground-squirrel and chicken orthologs of 11cRDH5. The predicted proteins were approximately 90% and 80% identical, respectively, to the human sequence. The second class of mRNAs encoded proteins that were only about 50% identical to 11cRDH5 from humans. In the one instance where comparisons of corresponding PCR products from the second class were possible, the ground-squirrel and chicken proteins were approximately 80% identical to each another. These data suggest the cloning of at least one new homolog of 11cRDH that is present in cone-dominant ground-squirrel and chicken retinas.

### EXAMPLE 11

### Novel All-trans-retinol Isomerase (atRI) and 11cRE-synthase in Cone-dominant Retinas

The retinyl-ester content of fresh bovine ocular tissues were analyzed. Bovine RPE contained approximately 95% all-trans-retinyl esters (atRE) and 5% 1cRE. No retinyl esters were detectable in bovine retinas. When [³H]atROL was added to microsomal membranes prepared from bovine RPE, the formation of [3H]atRE, and subsequent formation of [³H]11cROL were observed. Addition of all-trans-retinyl α-bromoacetate (tRBA), a potent inhibitor of LRAT (Trehan et al., 1990), completely suppressed the formation of both atRE and 11cROL. Addition of [³H]11cROL to membranes from bovine RPE resulted in the formation of [³H]11cRE. This reaction was also completely inhibited by tRBA. The isomeric inhibitor, 11-cis-retinyl α-bromoacetate (cRBA) was also synthesized. This agent suppressed the formation of [³H]11cRE from [³H]11cROL added to bovine-RPE membranes, but had no effect on the synthesis of [³H]atRE or [³H]11cROL from added [³H]atROL. No ester-synthase activity was detected in bovine retinas. The results of these studies on rod-dominant bovine eyes are in full accord with the published results of other investigators (Rando et al., 1992; Saari et al., 1994; Crouch et al., 1996; Trehan et al., 1990).

There are striking differences in retinyl-ester content and retinoid turnover upon analysis of ocular tissues from cone-dominant ground squirrel and chicken. Chicken retinas contained 60% 11cRE and 40% atRE, while ground-squirrel retinas contained 80% 11cRE and 20% atRE. The ratio of cones to rods is 60:40 in chicken (Meyer et al., 1973), 96:4 in ground squirrel (West et al., 1975), 3:97 in mouse (Carter-Dawson et al., 1979), and 8:92 in bovine retina (Krebs et al., 1982). Thus, across multiple species, the level of 11cRE in retina is loosely correlated with the ratio of cone to rod photoreceptors. Addition of [3H]atROL to membranes from chicken and ground squirrel retinas resulted in the formation of [³H]11cRE, [³H]atRE, and [³H]11cROL. Interestingly, addition of the tRBA inhibitor to these reaction mixtures completely suppressed formation of [3H]atRE, but had no effect on the formation of [³H]11cROL or [³H]11cRE. Addition of cRBA inhibitor also had no effect on synthesis of [³H]11cRE, in contrast to what was observed with bovine-RPE membranes. During time-course studies, [³H]11cROL always preceded the appearance of [3H]11cRE. These data indicate that ground-squirrel and chicken retinas contain an activity that isomerizes atROL to 11cROL by a different mechanism from IMH in RPE. In particular, this new all-trans-retinol isomerase (atRI) is insensitive to tRBA. This insensitivity rules out the trivial explanation of RPE contamination in retinal membrane preparations. Data also reveals the presence of a new 11cRE-synthase activity. Unlike LRAT, this new ester synthase is not inhibited by tRBA or cRBA, and has high specificity for the 11cROL substrate.

These results imply the existence of an alternative retinoid cycle for cone-pigment regeneration. The working model for this cone cycle is depicted in FIG. 1B. This pathway offers an explanation for the much faster regeneration kinetics observed in cone photoreceptors. Retinoid is stored as 11cRE after already undergoing the slow trans-to-cis isomerization step. Thus, 11cROL can be provided to cones for rapid pigment regeneration following simple ester hydrolysis in the Müller cell.

Prophetic Examples 12-16 can be used to test predictions of this hypothesis and provide information about the three new catalytic activities in cone-dominant retinas. The human macula contains the central fovea, which is exclusively populated with cone photoreceptors. The genes for these proteins may also be candidates for inherited diseases affecting cone photoreceptors including macular degeneration, as discussed.

### PROPHETIC EXAMPLE 12

### Biochemical Studies

Isomerization atROL to 11 cROL is accompanied by a free energy change of +4.1 kcal/mol (Rando et al., 1983). With the IMH-catalyzed reaction, this energy comes from the coupled hydrolysis of atROL fatty-acyl esters(ΔG=−5 kcal/mol) synthesized by LRAT. Inhibitor studies suggest that atRI acts by a different mechanism. The first issue to be addressed in the characterization of atRI is to define this source of energy. ATP hydrolysis is one possibility, perhaps involving a retinyl-phosphate intermediate. Alternatively, isomerization may proceed through a short-lived atRE intermediate, possibly generated by an activated fatty acid. A significant increase in the isomerization with addition of palmityl CoA to the reaction mixture corroborates this possibility. Thus, 11cRE-synthase may play a role in the isomerization of atROL by atRI, although the mechanism of this interaction must be unlike that of LRAT and IMH.

Data suggests that the 11cRDH detected in chicken and ground-squirrel retinas has a different K_{M} from that of 11cRDH5 in RPE. This difference can be verified with more detailed kinetic studies on crude membranes and partially purified 11cRDH from chicken retinas. Depending on the outcome of a PCR screen for a cone-form of 11cRDH, it may be necessary to purify this protein before cloning its mRNA. To begin purification of atRI, 11cRE-synthase, and possibly 11cRDH, one must first define a detergent/phospholipid mixture for solubilizing retinal membranes that preserve the catalytic activity of each enzyme. Recovery of 100% of atRI catalytic activity after solubilizing membranes from chicken retinas in either CHAPS or n-dodecyl maltoside detergents is possible. These detergent extracts can be fractionated by gel filtration, ion-exchange, and dye-affinity chromatography on an FPLC system. Assaying fractions simultaneously for all three enzymes may be possible. Purification of these proteins will be simplified by the abundance of available starting material (fresh chicken retinas). Analysis of peptide fragments from purified atRI, 11cRE-synthase, and possibly 11cRDH, by tandem mass-spectrometry and Edman degradation is also an aspect of determining K_{M}.

### PROPHETIC EXAMPLE 13

### Cloning of the Cone-11cRDH, atRI, and 11cRE-synthase mRNAs

cDNA fragments from ground-squirrel and chicken retina that appear to encode at least one homolog of 11cRDH at 50% identity to human 11cRDH5 have been identified from a PCR screen. This promising result suggests that cone-form of 11cRDH has been cloned. For atRI and 11cRE-synthase, the protein sequence information obtained in Section A to design degenerate oligonucleotides for amplification by PCR from chicken retinal cDNA. This is similar to the method used for cloning RmP (Azarian et al., 1997). For all three proteins, the full-length cDNAs can be isolated from mouse and human retinal libraries. Cells that express each mRNA by Northern blotting and in situ hybridization can be identified. Also, antibodies against 11cRDH, atRI, and 11cRE-synthase can be generated by expressing fragments of each protein in E. coli and/or by synthesizing peptides. These reagents can then be used for immunocytochemical localization of each protein in retina by light and electron microscopy, and in later functional studies.

### PROPHETIC EXAMPLE 14

### Evaluate the Genes for 11cRDH, atRI, and 11cRE-synthase as Candidates for Inherited Macular Degenerations

The chromosomal localization for each gene in mice and humans can be determined by radiation-hybrid analysis. If any locus maps to a site implicated in human macular or retinal disease, the gene can be cloned and its fine structure determined. This can be done in a manner similar to that of Travis et al., (1991), Ma et al., (1995) and Azarian et al. (1998) with RDS and ABCR. Mutations in affected humans can then be tested by clinical geneticists. If found, analysis of the effects of human mutations on the catalytic activity of 11cRDH, atRI, or 11cRE-synthase in a cell-expression system, described herein, can be done.

### PROPHETIC EXAMPLE 15

### Cell-expression Studies

Full-length clones of 11cRDH, atRI, and 11cRE-synthase can be expressed in HEK or baculovirus-infected Sf9 cells. The kinetic and substrate-specificity profiles for each expressed enzyme can be compared to those observed with crude retinal membranes and with the purified proteins. For example, membranes from cells expressing 11cRDH will be tested for the capacity to oxidize different retinoid substrates in the presence of appropriate cofactors. Samples will be analyzed by normal and reverse-phase HPLC using photodiode array and online radiometric-detection. Effects of interesting human mutations on expressed 11cRDH, atRI, or 11cRE-synthase will be examined, pending the results of experiments in Prophetic Example 6.

### PROPHETIC EXAMPLE 16

### Transgenic and Knockout Studies

A test to verify the cone visual cycle (FIG. 4) can be accomplished by generating transgenic mice that express cone 11cRDH in rod cells, using a rhodopsin promoter. The transgene will be placed on an rpe65−/− null genetic-background. Rods from these mice should use 11cROL secreted by Müller cells to regenerate visual pigment, restoring rod photosensitivity. Mice with a knockout mutation in the gene for 11cRDH can also be generated. These animals should manifest a reduction or complete loss of cone function by electroretinography. Double-homozygous (rpe65−/−; 11cRDH−/−) mice should lack both rod and cone visual pigments.

Mice with a knockout mutation in the gene for atRI can also be generated. According to the model, this enzyme is present in Müller cells, which have been shown to secrete 11cROL for uptake by cones (Das et al., 1992). Alternatively, atRI may be expressed in cones. In either case, inventors predict a phenotype in atRI-knockout mice of delayed cone recovery following light exposure, normal cone function in dark-adapted mice, and normal rod function. Double homozygotes can be made preferably by moving the atRI−/− mutation onto rpe65−/−, 11cRDH−/−, and abcr−/−(Weng et al., 1999) null-backgrounds in separate experiments.

If the genes for 11cRDH, atRI, or 11cRE-synthase are found to be affected in any human retinal diseases, a “virtual” mouse models of each disease will be made by duplicating the major alleles and predicted expression pattern. Depending on the results of Prophetic Experiment 5, these mice can be used for the development of new rational therapies as described by Travis et al., (1992), Kedzierski et al., (1997, 1998, 1999(1), and 1999(2)) and Nir et al. (2000), and Weng et al., (1999) in analysis of rds-transgenic and abcr−/− knockout mice.

### EXAMPLE 17

### Pharmacologic Treatment of the Retinopathy in abcr−/− Mice and Patients With STGD

Isotretinoin has been shown to inhibit 11cRDH in the RPE (Law et al., 1989; Gamble et al., 1999). Profound depletion of 11cRAL and atRAL is observed in outer segments and accumulation of 11cRE in RPE cells following intraperitoneal injection of isotretinoin into light-adapted wild-type mice. Long delays in 11cRAL regeneration are also observed after a photobleach in mice treated with isotretinoin. This alteration in the retinoid profile is similar to the pattern in 11cRDH knockout-mice (Driessen et al., 2000). No photoreceptor degeneration by histologic or electroretinographic (ERG) analysis was observed in mice following prolonged treatment with isotretinoin at doses up to 50 mg/kg/day (25×the maximum human dose).

### PROPHETIC EXAMPLE 18

### Effects of Isotretinoin on the Visual Cycle

The optimal dose of isotretinoin to block formation of A2E in abcr−/− mice can be determined. The recommended dose for treating acne is 0.5 to 2.0 mg/kg/day. The observation of occasional night blindness in humans suggests significant impairment of rhodopsin regeneration at normal therapeutic doses. Mice have been shown to tolerate isotretinoin at doses up to 400 mg/kg/day (Yuschak et al., 1993). The ID₅₀ (concentration required for 50% inhibition) of isotretinoin on 11cRDH is 150 Nm (Gamble et al., 1999). Isotretinoin was detected in retina and RPE by HPLC analysis five minutes following an intraperitoneal injection. Tissue concentrations in the micromolar range were observed one hour following a single injection of isotretinoin at 4 mg/kg. Based on these data, it appears that inhibitory concentrations of isotretinoin in RPE tissue should be achieved at doses similar to, or possibly below, human therapeutic doses for the treatment of acne. The effects of isotretinoin on atRAL in retinas from light-adapted mice would preferably be determined at doses that bracket the human therapeutic dose. The serum half-life of isotretinoin in humans is 10-20 hours (Hoffinann-La Roche, product information for Accutane). The preferred method includes treating mice with a single morning intraperitoneal dose. An increased frequency of injections may be required to maintain reduced levels of atRAL in the retina throughout the day.

### PROPHETIC EXAMPLE 19

### Treatment of A2E Accumulation and Photoreceptor Degeneration in abcr−/− Mice With Isotretinoin

Testing isotretinoin as an inhibitor of photoreceptor degeneration in abcr−/− mice can be accomplished as follows. Since, photoreceptor degeneration in abcr−/− mice, and presumably humans with STGD, is secondary to A2E-mediated poisoning of the RPE, A2PE-H₂ and A2E can be readily measured in abcr−/− mice at three weeks but will remain undetectable in wild-type mice until approximately eight months. Examining the effects of isotretinoin on A2PE-H₂ and A2E levels in abcr−/− retina and RPE can then be accomplished, respectively. An inhibitory effect of isotretinoin on A2E accumulation will be corroborated by electron microscopic analysis of RPE from abcr−/− mice. Previously, the inventors have shown substantial ultrastructural changes in abcr−/− RPE including the presence of many electron-dense lipofuscin granules (Weng et al., 1999).

An aspect of the invention is to slow photoreceptor degeneration in abcr−/− mice by treatment with isotretinoin. This will be a crucial observation, since blindness in STGD results from photoreceptor death not lipofuscin accumulation. The rate of cell death in pigmented abcr−/− mice is slow, with one-third of photoreceptors lost during the first year. To accelerate the rate of data acquisition, the abcr−/− null-mutation can be moved onto an albino (BALB/c) background. The albino background is sensitizing for multiple retinal degenerations in mice. For example, the rate of photoreceptor loss in albino retinal degeneration slow (rds−/−) mice is approximately twice that of pigmented rds−/− mice (Sanya et al., 1986). However, no degeneration of photoreceptors is seen in wild-type albino mice raised under normal cyclic lighting. Rates of photoreceptor degeneration can be monitored in treated and untreated wild-type and abcr−/− mice by two techniques. One is the study of mice at different times by ERG analysis as described by Weng et al (1999) and Kedzierski et al. (1997) and is adopted from a clinical diagnostic procedure. An electrode is placed on the corneal surface of an anesthetized mouse and the electrical response to a light flash is recorded from the retina. Amplitude of the a-wave, which results from light-induced hyperpolarization of photoreceptors (Granit et al., 1947), is a sensitive indicator of photoreceptor degeneration (Kedzierski et al., 1997). ERGs are done on live animals. The same mouse can therefore be analyzed repeatedly during a time-course study. The definitive technique for quantitating photoreceptor degeneration is histological analysis of retinal sections. The number of photoreceptors remaining in the retina at each time point will be determined by counting the rows of photoreceptor nuclei in the outer nuclear layer.

### PROPHETIC EXAMPLE 20

### Treatment of Human Patients With STGD

Physiologically, ultrastructurally, and biochemically, the clinical phenotype in humans with STGD closely resembles that observed in abcr−/− mice (Fishman et al., 1991; Birnbach et al., 1994; Weng et al., 1999; Mata et al., 2000; Eagle et al., 1980). If lipofuscin deposition in RPE cells and degeneration of photoreceptors can be inhibited in abcr−/− mice by treatment with isotretinoin, it is likely that a therapeutic effect will also be observed in humans with STGD. Since the ultimate goal of therapy for patients with STGD is to preserve visual acuity, visual acuity is the preferred outcome measure. This parameter can be simply and reliably ascertained. Patients will also be followed by ERG. Isotretinoin is contraindicated during pregnancy due to the possibility of birth defects. The study will preferably be a randomized, double-blind, and placebo controlled. A 50% reduction in the rate of visual loss in STGD patients treated with isotretinoin would represent a major treatment breakthrough. Even if the visual loss eventually “catches up” during later life, an important benefit may still be realized in these patients, having preserved vision during the important educational years.

### EXAMPLE 21

### Cloning a Novel 11-cis-Retinol Dehydrogenase from Bovine Retinal Pigment Epithelium

A novel 11-cis-retinol dehydrogenase was cloned from bovine retinal pigment epithelium. Standard cloning techniques were used, and can be found in, for example, Sambrook et al. (1989). Amino acid sequences of previously known dehydrogenases that are related to 11-cis-retinol dehydrogenase type-5 (11cRDH5) were aligned and the conserved regions between the different dehydrogenases were found. A highly conserved region is the cofactor binding region and is believed to be involved in the conversion of 11 cis-retinol to 11-cis-retinaldehyde. These conserved regions were used to design degenerate oligonucleotide-primers, which are identified as SEQ ID NO: 25 (sense) and SEQ ID NO: 26 (antisense). PCR was done with each primer set on template consisting of first-stand cDNA synthesized from poly(A)+ mRNA prepared from fresh bovine retinal pigment epithelial tissue. The PCR product was then used to screen the library consisting of the dehydrogenases related to 11cRDH5. A positive clone was identified, as is provided is SEQ ID NO: 3. A Northern blot analysis of SEQ ID NO: 3 verified cell expression.

Further cloning attempts, using different primers and/or slightly different methods, can be done to clone other novel dehydrogenases that are able to recognize and oxidize 11 cis-retinol. Any of the primers, directed to conserved regions, may be used to screen cDNA or genomic mammalian libraries, as described herein. It is contemplated that these primers may also be used to amplify DNA segments that encod all or part of an 11cRD.

### EXAMPLE 22

### Administration of Isotretinoin Inhibits Formation of Lipofuscin

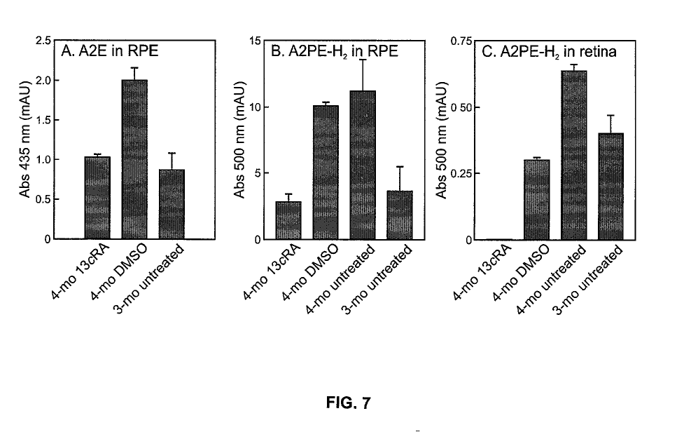

Three-month-old abcr−/− mice were treated with isotretinoin (13 cis retinoic acid) for one month. FIG. 7 shows the result of this treatment on levels the lipofuscin fluorophore, A2E, and its precursor, A2PE-H₂. As depicted in FIG. 7A, A2E in RPE from four-month-old abcr−/− mice treated with isotretinoin was approximately 50% the level in sham-treated (DMSO) controls, and similar to the level in untreated three-month-old abcr−/− mice. An even larger difference in levels of the A2E-precursor, A2PE-H₂, were observed in four-month-old abcr−/− RPE after one month of treatment with isotretinoin (FIG. 7B). Presumably, this reflects the geometric increase of A2PE-H₂ observed with age in abcr−/− RPE. Strikingly, A2PE-H₂ was virtually undetectable in four-month-old abcr−/− retinas after one month of treatment with isotretinoin (FIG. 7C). Since A2PE-H₂ synthesized in the retina is the source of A2PE-H₂ and A2E in the RPE (Mata et al., 2000), this result suggests that treatment with isotretinoin may completely block new formation of A2E. These results show that that inhibiting 11-cis-retinol dehydrogenase is a mechanism to block lipofuscin accumulation in patients with early recessive Stargardt's disease and potentially, age-related macular degeneration.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

Abbondanzo et al., Breast Cancer Res. Treat., 16:182(#151), 1990.

Abbondanzo et al., Am J Clin Pathol. 93(5):698-702, 1990.

Abbondanzo et al., Am J Pediatr Hematol Oncol. 12(4):480-9, 1990.

Affymax technology; Bellus, 1994

Ahn, J., Wong, J. T. & Molday, R. S. The effect of lipid environment and retinoids on the ATPase activity of ABCR, the photoreceptor transporter responsible for Stargardt macular dystrophy. Journal of Biological Chemistry 275, 20399-20405 (2000).

Alexander, K. R., and Fishman, G. A. (1984). Prolonged rod dark adaptation in retinitis pigmentosa. Br. J. Ophthalmol. 68, 561-569.

Allikmets, R. & Consortium, I. A. S. Further Evidence for an Association of ABCR Alleles with Age-related Macular Degeneration. Am. J. Hum. Genet 67, 487-491 (2000).

Allikmets, R., Shroyer, N. F., Singh, N., Seddon, J. M., Lewis, R. A., Bernstein, P. S., Peiffer, A., Zabriskie, N. A., Li, Y., Hutchinson, A., Dean, M., Lupski, J. R., and Leppert, M. (1997). Mutation of the Stargardt disease gene (ABCR) in age-related macular degeneration. Science 277, 1805-1807.

Allikmets, R., Singh, N., Sun, H., Shroyer, N. F., Hutchinson, A., Chidambaram, A., Gerrard, B., Baird, L., Stauffer, D., Peiffer, A., Rattner, A., Smallwood, P., Li, Y., Anderson, K. L., Lewis, R. A., Nathans, J., Leppert, M., Dean, M., and Lupski, J. R. (1997). A photoreceptor cell-specific ATP-binding transporter gene (ABCR) is mutated in recessive Stargardt macular dystrophy. Nat. Genet. 15, 236-246.

Allred et al., Breast Cancer Res. Treat., 16:182(#149), 1990.

Allred et al., Arch Surg. 125(1):107-13, 1990.

Almendro N, Bellon T, Rius C, Lastres P, et al. Cloning of the human platelet endothelial cell adhesion molecule-1 promoter and its tissue-specific expression. Structural and functional characterization. J Immunol Dec. 15, 1996;157(12):5411-21.

Anderson, R. E., and Maude, M. B. (1970). Phospholipids of bovine outer segments. Biochemistry 9, 3624-3628.

Angel et al., Cell, 49:729, 1987b.

Angel et al., Mol. Cell. Biol., 7:2256, 1987a.

Atchison and Perry, Cell, 46:253, 1986.

Atchison and Perry, Cell, 48:121, 1987.

Atherton et al., Biol. of Reproduction, 32, 155-171, 1985.

Azarian, S. M., Megarity, C. F., Weng, J., Horvath, D. H., and Travis, G. H. (1998). The human photoreceptor rim protein gene (ABCR)-genomic structure and primer set information for mutation analysis. Hum. Genet. 102, 699-705.

Azarian, S. M. & Travis, G. H. The Photoreceptor Rim Protein Is an ABC Transporter Encoded By the Gene For Recessive Stargardts-Disease (ABCR). FEBS Letters 409, 247-252 (1997).

Banerji et al., Cell, 27:299, 1981.

Banerji et al., Cell, 35:729, 1983.

Berberian et al., Science, 261:1588-1591, 1993.

Bergink G J, Hoyng C B van der Maazen R W M, et al. A randomized controlled clinical trial on the efficacy of radiation therapy in the control of subretinal choroidal neovascularization in age-related macular degeneration: radiation versus observation. Graefes Arch Clin Exp Ophthalmol 1998;236:321-325.

Bergsma, D. R., Wiggert, B. N., Funahashi, M., Kuwabara, T., and Chader, C. J. (1977). Vitamin A receptors in normal and dystrophic human retina. Nature 265, 66-67.

Berkhout et al., Cell, 59:273, 1989.

Berkow J W, Kelley J S, and Orth D H, “Fluorescein Angiography: A Guide to the Interpretation of Fluorescein Angiograms,” American Academy Ophthalmology, San Francisco, Calif.: 1984.

Berzal-Herranz, A. et al., Genes and Devel., 6:129-134, 1992.

Birch, D. G., and Hood, D. C. (1995). Abnormal rod photoreceptor function in retinitis pigmentosa. In Degenerative Diseases of the Retina, R. Anderson, J. Hollyfield and M. LaVail, eds. (New York: Plenum), pp. 359-369.

Birnbach, C. D., Jarvelainen, M., Possin, D. E. & Milam, A. H. Histopathology and immunocytochemistry of the neurosensory retina in fundus flavimaculatus. Ophthalmology 101, 1211-9 (1994).

Blacharski, P. A. Fundus flavimaculutus., 135-159 (Raven Press, New York, 1988).

Blanar et al., EMBO J, 8:1139, 1989.

Bligh, D. G., and Dyer, W. J. (1959). A rapid method for total lipid extraction and purification. Can. J. Biochem. Physiol. 37, 911-917.

Bliss, A. F. (1948) J. Biol. Chem. 172, 165-178.

Bodine and Ley, EMBO J., 6:2997, 1987.

Boshart et al., Cell, 41:521, 1985.

Bosze et al., EMBO J., 5:1615, 1986.

Braddock et al., Cell, 58:269, 1989.

Bressler N M, Bressler S B, Fine S L. Age related macular degeneration. Surv Ophthalmol 1988; 32: 375-413.

Bressler N M, Bressler S B, Gragoudas E S. Clinical characteristics of choroidal neovascular membranes. Arch Ophthlamol 1987; 105: 209-213.

Bressler N M, Gills J P. Age related macular degeneration. New hope for a common problem comes from photodynamic therapy. BMJ Dec. 9, 2000;321(7274):1425-1427.

Brown et al., Breast Cancer Res. Treat., 16:192(#191), 1990.

Brown et al., J Immunol Methods., 12;130(1):111-121, 1990.

Bulla and Siddiqui, J. Virol., 62:1437, 1986.

Bunt-Milam, A. H. & Saari, J. C. Immunocytochemical localization of two retinoid-binding proteins in vertebrate retina. Journal of Cell Biology 97, 703-12 (1983).

Campbell and Villarreal, Mol. Cell. Biol., 8:1993, 1988.

Campere and Tilghman, Genes and Dev., 3:537, 1989.

Campo et al., Nature, 303:77, 1983.

Carter-Dawson, L. D. & LaVail, M. M. Rods and cones in the mouse retina. I. Structural analysis using light and electron microscopy. J Comp Neurol 188, 245-62 (1979).

Cech et al., Cell, 27:487-496, 1981.

Celander and Haseltine, J. Virology, 61:269, 1987.

Celander et al., J. Virology, 62:1314, 1988.

Chandler et al., Cell, 33:489, 1983.

Chang et al., Mol. Cell. Biol., 9:2153, 1989.

Chatterjee et al., Proc. Nat'l Acad. Sci. USA., 86:9114, 1989.

Chen, H., and Anderson, R. E. (1992). Quantitation of phenacyl esters of retinal fatty acids by high-performance liquid chromatography. J. Chromatogr. 578, 124-129.

Choi et al., Cell, 53:519, 1988.

Chowrira, B. H. et al., Biochemistry, 32:1088-1095, 1993.

Chowrira, B. H. et al., J. Biol. Chem., 269:25856-25864, 1994.

Churchill, P., Hempel, J., Romovacek, H., Zhang, W. W., Churchill, S., and Brennan, M. (1992) Biochemistry 31, 3793-3799

Cleary et al., Trends Microbiol., 4:131-136, 1994.

Cohen et al., “A Repetitive Sequence Element 3′ of the human c-Ha-ras1 Gene Has Enhancer Activity,” J. Cell. Physiol., 5:75, 1987.

Costa et al., Mol. Cell. Biol., 8:81, 1988.

Cremers, F. P. M., van de Pol, D. J. R., van Driel, M., den Hollander, A. I., van Haren, F. J. J., Knoers, N. V. A. M., Tijmes, N., Bergen, A. A. B., Rohrschneider, K., Blankenagel, A., Pinckers, A. J. L. G., Deutman, A. F., and Hoyng, D. B. (1998). Autosomal recessive retinitis pigmentosa and cone-rod dystrophy caused by splice site mutations in the Stargardt's disease gene ABCR. Hum. Mol. Genet. 7, 355-362.

Cripe et al., EMBO J., 6:3745, 1987.

Crouch, R. K., Chader, G. J., Wiggert, B. & Pepperberg, D. R. Retinoids and the visual process. Photochemistry & Photobiology 64, 613-21 (1996).

Culotta and Hamer, Mol. Cell. Biol., 9:1376, 1989.

Daemen, F. J., and Bonting, S. L. (1969). Internal protonation in retinylidene phosphatidylethanolamine and the red-shift in rhodopsin. Nature 222, 879-881.

Dagnelie G, Zorge I S, McDonald T M. Lutein improves visual function in some patients with retinal degeneration: a pilot study via the Internet. Optometry 2000 March;71(3):147-64.

Dandolo et al., J. Virology, 47:55, 1983.

Das, S. R., Bhardwaj, N., Kjeldbye, H. & Gouras, P. Muller cells of chicken retina synthesize 11-cis-retinol. Biochemical Journal 285, 907-13 (1992).

De Jager R, Abdel-Nabi H, Serafmi A, Pecking A, Klein J L, Hanna M G Jr., “Current status of caner immunodetection with radiolabeled human monoclonal antibodies” Semin Nucl Med 23(2):165-179, 1993.

De La Paz, M. A. et al. Analysis of the Stargardt disease gene (ABCR) in age-related macular degeneration. Ophthalmology 106, 1531-6 (1999).

De Laey, J. J. & Verougstraete, C. Hyperlipofuscinosis and subretinal fibrosis in Stargardt's disease. Retina 15, 399-406 (1995).

De Villiers et al., Nature, 312:242, 1984.

Decottignies, A., Grant, A. M., Nichols, J. W., de Wet, H., McIntosh, D. B., and Goffeau, A. (1998). ATPase and multidrug transport activities of the overexpressed yeast ABC protein Yor1p. J. Biol. Chem. 273, 12612-12622.

Delori, F. C., Fleckner, M. R., Goger, D. G., Weiter, J. J. & Dorey, C. K. Autofluorescence Distribution Associated with Drusen in Age-related Macular Degeneration. Invest. Ophthalmol. Vis. Sci. 41, 496-504 (2000).

DeMar, J. C., Jr., Wensel, T. G. & Anderson, R. E. (1996) J. Biol. Chem. 271, 5007-16.

Deschamps et al., Science, 230:1174, 1985.

Dholakia et al., J. Biol. Chem., 264, 20638-20642, 1989.

Dogra, S., Krishnamurthy, S., Gupta, V., Dixit, B. L., Gupta, C. M., Sanglard, D., and Prasad, R. (1999). Asymmetric distribution of phosphatidylethanolamine in C-albicans: possible mediation by CDR1, a multidrug transporter belonging to ATP binding cassette (ABC) superfamily. Yeast 15, 111-121.

Doolittle M H and Ben-Zeev O, “Immunodetection of lipoprotein lipase: antibody production, immunoprecipitation, and western blotting techniques” Methods Mol Biol., 109:215-237, 1999.

Dorey, C. K., Wu, G., Ebenstein, D., Garsd, A. & Weiter, J. J. Cell loss in the aging retina. Relationship to lipofuscin accumulation and macular degeneration. Invest. Ophthalmol. Vis. Sci. 30, 1691-9 (1989).

Driessen, C. et al. Disruption of the 11-cis-retinol dehydrogenase gene leads to accumulation of cis-retinols and cis-retinyl esters. Molecular & Cellular Biology 20, 4275-4287 (2000).

Eagle, R. C., Jr., Lucier, A. C., Bernardino, V. B., Jr., and Yanoff, M. (1980). Retinal pigment epithelial abnormalities in fundus flavimaculatus: a light and electron microscopic study. Ophthalnology 87, 1189-1200.

Edbrooke et al., Mol. Cell. Biol., 9:1908, 1989.

Edlund et al., Science, 230:912, 1985.

Egholm et al., Nature 1993, 365, 566

Eldred, G. E. (1995). Lipofuscin fluorophore inhibits lysosomal protein degradation and may cause early stages of macular degeneration. Gerontology 41, 15-28.

Eldred, G. E., and Katz, M. L. (1988). Fluorophores of the human retinal pigment epithelium: separation and spectral characterization. Exp. Eye Res. 47, 71-86.

Eldred, G. E. & Lasky, M. R. Retinal age pigments generated by self-assembling lysosomotropic detergents. Nature 361, 724-6 (1993).

EP 266,032, or via deoxynucleoside H-phosphonate intermediates as described by Froehler et al., Nucl. Acids Res., 14:5399-5407, 1986,

European Application No. 320 308

European Application No. 329 822

Fain, G. L., Matthews, H. R., and Cornwall, M. C. (1996). Dark adaptation in vertebrate photoreceptors. Trends Neurosci. 19, 502-507.

Feeney-Burns, L., and Eldred, G. E. (1983). The fate of the phagosome: conversion to ‘age pigment’ and impact in human retinal pigment epithelium. Trans. Ophthalmol. Soc. U.K. 103, 416-421.

Feng and Holland, Nature, 334:6178, 1988.

Finger P T, Chakravarthy U. Radiotherapy as treatment for age-related macular degeneration. Arch Ophthalmol 116:1507-1509, 1998.

Firak and Subramanian, Mol. Cell. Biol., 6:3667, 1986.

Fishman, G. A., Pulluru, P., Alexander, K. R., Derlacki, D. J., and Gilbert, L. D. (1994). Prolonged rod dark adaptation in patients with cone-rod dystrophy. Am. J. Ophthalmol. 118, 362-367.

Fishman, G. A., Farbman, J. S. & Alexander, K. R. Delayed rod dark adaptation in patients with Stargardt's disease. Ophthalmology 98, 957-62 (1991).

Fliesler, S. J., and Anderson, R. E. (1983). Chemistry and metabolism of lipids in the vertebrate retina. Prog. Lipid Res. 22, 79-131.

Foecking and Hofstetter, “Powerful and/or Versatile Enhancer-Promoter Unit for [mammalian, plant, fungus, bacteria?] Expression Vectors,” Gene, 45:101, 1986.

Forster and Symons, Cell, 49:211-220, 1987.

Frohman, In: PCR PROTOCOLS: A GUIDE TO METHODS AND APPLICATIONS, Academic Press, N.Y., 1990.

Fujita et al., Cell, 49:357, 1987.

Gamble, M. V. et al. Biochemical properties, tissue expression, and gene structure of a short chain dehydrogenase/reductase able to catalyze cis-retinol oxidation. Journal of Lipid Research 40, 2279-92 (1999).

GB Application No. 2 202 328

Gerlach et al., Nature (London), 328:802-805, 1987.

Gilles et al., Cell, 33:717, 1983.

Gloss et al., EMBO J, 6:3735, 1987.

Godbout et al., Mol. Cell. Biol., 8:1169, 1988.

Goldstein, E. B. & Wolf, B. M. Regeneration of the Green-rod Pigment in the Isolated Frog Retina. Vision Research 13, 527-534 (1973).

Goldstein, E. B. Early Receptor Potential of the Isolated Frog (Rana pipiens) Retina. Vision Research 7, 837-845 (1967).

Gonzalez-Fernandez, F. et al 11-cis Retinol dehydrogenase mutations as a major cause of the congenital night-blindness disorder known as fundus albipunctatus. Molecular Vision 5, NIL_{—}1-NIL 6 (1999).

Goodbourn and Maniatis, Proc. Nat'l Acad. Sci. USA, 85:1447, 1988.

Goodbourn et al., Cell, 45:601, 1986.

Granit, R. (1947). Sensory Mechanism of the Retina (London: Oxford University Press).

Greene et al., Immunology Today, 10:272, 1989.

Groenendijk, G. W., De Grip, W. J., and Daemen, F. J. (1984). Rod outer segment membranes: good acceptors for retinoids? Vision Res. 24, 1623-1627.

Grosschedl and Baltimore, Cell, 41:885, 1985.

Gulbis B and Galand P, “Immunodetection of the p21-ras products in human normal and preneoplastic tissues and solid tumors: a review” Hum Pathol 24(12):1271-1285, 1993.

Haeseleer, F., Huang, J., Lebioda, L., Saari, J. C., Palczewski, K. (1998). Molecular Characterization of a Novel Short-chain Dehydrogenase/Reductase That Reduces All-trans-retinal. J. Biol. Chem. 273: 21790-21799.

Hasan T, Schmidt-Erfurth U. Mechanisms of action of photodynamic therapy with verteporfin for the treatment of age-related macular degeneration. Surv Ophthalmol (in press).

Haslinger and Karin, Proc. Nat'l Acad. Sci. USA., 82:8572, 1985.

Hauber and Cullen, J. Virology, 62:673, 1988.

Hawkins, B. S., Bird, A., Klein, R. & West, S. K. Epidemiology of age-related macular degeneration. Molecular Vision 5, NIL_{—}7-NIL_{—}10 (1999).

Heiba, I. M., Elston, R. C., Klein, B. E. & Klein, R. Sibling correlations and segregation analysis of age-related maculopathy: the Beaver Dam Eye Study [published erratum appears in Genet Epidemiol 1994; 11(6):571]. Genetic Epidemiology 11, 51-67 (1994).

Hen et al., Nature, 321:249, 1986.

Hensel et al., Lymphokine Res., 8:347, 1989.

Herr and Clarke, Cell, 45:461, 1986.

Hirochika et al., J. Virol., 61:2599, 1987.

Hirsch et al., Mol. Cell. Biol., 10:1959, 1990.

Holbrook et al., Virology, 157:211, 1987.

Holz, F. G., Schutt, F., Kopitz, J., Eldred, G. E., Kruse, F. E., Volcker, H. E., and Cantz, M. (1999). Inhibition of lysosomal degradative functions in RPE cells by a retinoid component of lipofuscin. Invest. Ophthalmol. Vis. Sci. 40, 737-743.

Holz, F. G. et al. Inhibition of lysosomal degradative functions in RPE cells by a retinoid component of lipofuscin. Invest. Ophthalmol. Vis. Sci. 40, 737-743 (1999).

Hood, D. C., and Birch, D. G. (1993). Human cone receptor activity: the leading edge of the a-wave and models of receptor activity. Vis. Neurosci. 10, 857-871.

Hood, D. C. & Hock, P. A. Recovery of cone receptor activity in the frog's isolated retina. Vision Research 13, 1943-51 (1973a).

Hood, D. C., Hock, P. A. & Grover, B. G. Dark adaptation of the frog's rods. Vision Research 13, 1953-63 (1973b).

Horlick and Benfield, Mol. Cell. Biol., 9:2396, 1989.

Huang et al., Cell, 27:245, 1981.

Hubbard, R., Brown, P. K., and Bownds, D. (1971). Methodology of vitamin A and visual pigments. Methods Enzymol. 18C, 615-653.

Hug H, Costas M, Staeheli P, Aebi M, et al. Organization of the murine Mx gene and characterization of its interferon- and virus-inducible promoter. Mol Cell Biol 1988 August;8(8):3065-79.

Hwang et al., Mol. Cell. Biol., 10:585, 1990.

Illing, M., Molday, L. L., and Molday, R. S. (1997). The 220-kDa rim protein of retinal rod outer segments is a member of the ABC transporter superfamily. J. Biol. Chem. 272, 10303-10310.

Imagawa et al., Cell, 51:251,1987.

Imbra and Karin, Nature, 323:555, 1986.

Imler et al., Mol. Cell. Biol., 7:2558, 1987.

Innis et al., “DNA sequencing with Thermus aquaticus DNA polymerase and direct sequencing of polymerase chain reaction-amplified DNA,” Proc Natl Acad Sci U.S.A. 85(24):9436-9440, 1988.

Jager, S., Palczewski, K., and Hoffmann, K. P. (1996). Opsin/all-trans-retinal complex activates transducin by different mechanisms than photolyzed rhodopsin. Biochemistry 35, 2901-2908.

Jakobovits et al., Mol. Cell. Biol., 8:2555, 1988.

Jameel and Siddiqui, Mol. Cell. Biol., 6,710, 1986.

Jaynes et al., Mol. Cell. Biol., 8:62, 1988.

Jin, J., Jones, G. J., Cornwall, M. C. (1994). Movement of retinal along cone and rod photoreceptors [published erratum appears in Vis. Neurosci. (1994). 11, 838]. Vis. Neurosci. 11, 389-399.

Johnson et al., Mol. Cell. Biol., 9:3393, 1989.

Jonas, J. B., Schneider, U. & Naumann, G. O. Count and density of human retinal photoreceptors. Graefes Archive for Clinical & Experimental Ophthalmology 230, 505-10 (1992).

Jones, G. J., Grouch, R. K., Wiggert, B., Cornwall, M. C. & Chader, G. J. Retinoid requirements for recovery of sensitivity after visual-pigment bleaching in isolated photoreceptors Proc. Natl. Acad. Sci. USA 86, 9606-10 (1989).

Joyce, Nature, 338:217-244, 1989.

Kadesch and Berg, Mol. Cell. Biol., 6:2593, 1986.

Kang et al., Science 240:1034-1036, 1988.

Karin et al., Mol. Cell. Biol., 7:606, 1987.

Katinka et al., Cell, 20:393, 1980.

Katinka et al., Nature, 290:720, 1981.

Katz, M. L., Rice, L. M. & Gao, C. L. (1999) Invest. Ophthalmol. Vis. Sci. 40, 175-181.

Kawamoto et al., Mol. Cell. Biol., 8,267, 1988.

Kedzierski, W., Bok, D., and Travis, G. H. (1999). Transgenic analysis of rds/peripherin N-glycosylation: effect on dimerization, interaction with rom1, and rescue of the rds null-phenotype. J. Neurochem. 72, 430-438.

Kedzierski, W., Lloyd, M., Brich, D. G., Bok, D. & Travis, G. H. Generation and Analysis of Transgenic Mice Expressing P216L-substituted Rds/Peripherin in Rod Photoreceptors. Invest. Ophthalmol. Vis. Sci. 38, 498-509 (1997).

Khatoon et al., Ann. of Neurology, 26, 210-219, 1989.

Kiledjian et al., Mol. Cell. Biol., 8:145, 1988.

Kim and Cech, Proc. Nat'l Acad. Sci. USA, 84:8788-8792, 1987.

King et al., J. Biol. Chem., 269, 10210-10218, 1989.

Klamut et al., Mol. Cell. Biol., 10: 193, 1990.

Klaver, C. C. et al. Genetic risk of age-related maculopathy. Population-based familial aggregation study. Archives of Ophthalmology 116, 1646-51 (1998).

Klein, M. L., Mauldin, W. M. & Stoumbos, V. D. Heredity and age-related macular degeneration. Observations in monozygotic twins. Archives of Ophthalmology 112, 932-7 (1994).

Kliffen, M., van der Schaft, T. L., Mooy, C. M., and de Jong, P. T. (1997). Morphologic changes in age-related maculopathy. Microsc. Res. Tech. 36, 106-122.

Kliffen, M., van der Schaft, T. L., Mooy, C. M. & de Jong, P. T. Morphologic changes in age-related maculopathy. Microscopy Research & Technique 36, 106-22 (1997).

Koch et al., Mol. Cell. Biol., 9:303, 1989.

Kohler et al., Methods Enzymol., 178:3, 1989.

Kolb, H., and Gouras, P. (1974). Electron microscopic observations of human retinitis pigmentosa, dominantly inherited. Invest. Ophthalmol. 13, 487-498.

Kornberg and Baker, DNA Replication, 2nd Ed., Freeman, San Francisco, 1992.

Kraus J, Woltje M, Schonwetter N, Hollt V. Alternative promoter usage and tissue specific expression of the mouse somatostatin receptor 2 gene. FEBS Lett May 29, 1998; 428(3):165-70.

Krebs, W. & Krebs, I. P. Quantitative Morphology of the Bovine Eye. in The Structure of the Eye (ed. Hollyfield, J. G.) 175-182 (Elsevier Biomedical, New York, 1982).

Kreier et al., Infection, Resistance and Immunity, Harper & Row, New York, (1991)).

Kriegler and Botchan, In: Eukaryotic Viral Vectors, Y. Gluzman, ed., Cold Spring Harbor: Cold Spring Harbor Laboratory, NY, 1982.

Kriegler and Botchan, Mol. Cell. Biol., 3:325, 1983.

Kriegler et al., Cell, 38 :483, 1984a.

Kriegler et al., Cell, 53:45, 1988.

Kriegler et al., In: Cancer Cells 2/Oncogenes and Viral Genes, Van de Woude et al. eds, Cold Spring Harbor, Cold Spring Harbor Laboratory, 1984b.

Kriegler et al., In: Gene Expression, D. Hamer and M. Rosenberg, eds., New York: Alan R. Liss, 1983.

Kuhl et al., Cell, 50:1057, 1987.

Kunz et al., Nucl. Acids Res., 17:1121, 1989.

Kwoh, et al., “Transcription-based amplification system and detection of amplified human immunodeficiency virus type 1 with a bead-based sandwich hybridization format, Proc Natl Acad Sci U.S.A. 86(4):1173-1177, 1989.

Lareyre J J, Thomas T Z, Zheng W L, Kasper S, et al. A 5-kilobase pair promoter fragment of the murine epididymal retinoic acid-binding protein gene drives the tissue-specific, cell-specific, and adrogen-regulated expression of a foreign gene in the epididymis of transgenic mice. J Biol Chem Mar. 19, 1999; 274(12):8282-90.

Larsen et al., Proc. Nat'l Acad. Sci. USA., 83:8283, 1986.

Laspia et al., Cell, 59:283, 1989.

Latimer et al., Mol. Cell. Biol., 10:760, 1990.

Law, W. C. & Rando, R. R. The molecular basis of retinoic acid induced night blindness. Biochemical & Biophysical Research Communications 161, 825-9 (1989).

Lee et al., Mol. Endocrinol., 2: 44-411, 1988.

Lee et al., Nature, 294:228, 1981.

Lee S H, Wang W, Yajima S, Jose P A, et al. Tissue-specific promoter usage in the D1A dopamine receptor gene in brain and kidney. DNA Cell Biol 1997 November;16(11):1267-75.

Lee, B. L., and Heckenlively, J. R. (1999). Stargardt's disease and fundus flavimaculatus. in Retina-Vitreous-Macula, D. R. GUyer, L. A. Yamuzzi, S. Chang, J. A. Shields and W. R. Green, eds.: W. B. Saunders), pp. 978-988.

Leibrock, C. S., Reuter, T., and Lamb, T. D. (1998). Molecular basis of dark adaptation in rod photoreceptors. Eye 12, 511-520.

Lenert et al., Science, 248:1639-1643, 1990.

Levinson et al., Nature, 295:79, 1982.

Lewis, R. A., Shroyer, N. F., Singh, N., Allikmets, R., Hutchinson, A., Li, Y., Lupski, J. R., Leppert, M., and Dean, M. (1999). Genotype/phenotype analysis of a photoreceptor-specific ATP-binding cassette transporter gene, ABCR, in Stargardt disease. Am. J. Hum. Genet. 64, 422-434.

Lewis, R. A. et al. Genotype/Phenotype Analysis of a Photoreceptor-Specific ATP-Binding Cassette Transporter Gene, ABCR, in Stargardt Disease Am. J. Hum. Genet. 64, 422-434 (1999).

Lieber and Strauss, Mol. Cell. Biol., 15: 540-551, 1995.

Lin et al., Mol. Cell. Biol., 10:850, 1990.

Luria et al., EMBO J., 6:3307, 1987.

Lusky and Botchan, Proc. Nat'l Acad. Sci. USA., 83:3609, 1986.

Lusky et al., Mol. Cell. Biol., 3:1108, 1983.

Luu-The, V., Labrie, C., Zhao, H. F., Couet, J., Lachance, Y., Simard, J., Leblanc, G., and Labrie, F. (1989) Mol. Endocrinol. 3, 1301-1309

Lyubarsky, A. L., and Pugh, E. N., Jr. (1996). Recovery phase of the murine rod photoresponse reconstructed from electroretinographic recordings. J. Neurosci. 16, 563-571.

Macular Photocoagulation Study Group. Argon laser photocoagulation for neovascular maculopathy after five years: Results from randomized clinical trials. Arch Ophthalmol 1991(a); 109: 1109-1114.

Macular Photocoagulation Study Group. Krypton laser photocoagulation for neovascular lesions of age-related macular degeneration. Arch Ophthalmol 1991(b); 109: 614-615.

Macular Photocoagulation Study Group. Laser photocoagulation of subfoveal neovascular lesions of age-related macular degeneration: Updated findings from two clinical trials. Arch Ophthalmol 1993; 111: 1200-1209.

Majors and Varmus, Proc. Nat'l Acad. Sci. USA., 80:5866, 1983.

Martinez-Mir, A., Paloma, E., Allikmets, R., Ayuso, C., Delrio, T., Dean, M., Vilageliu, L., Gonzalezduarte, R., and Balcells, S. (1998). Retinitis pigmentosa caused by a homozygous mutation in the Stargardt-disease gene ABCR. Nat. Genet. 18, 11-12.

Mata, N. L., Weng, J. & Travis, G. H. Biosynthesis of a Major Lipofuscin Fluorophore in Mice and Humans with ABCR-mediated Retinal and Macular Degeneration. Proc. Natl. Acad. Sci. USA 97, 7154-7159 (2000).

McNeall et al., Gene, 76:81, 1989.

Melia, T. J., Jr., Cowan, C. W., Angleson, J. K., and Wensel, T. G. (1997). A comparison of the efficiency of G protein activation by ligand-free and light-activated forms of rhodopsin. Biophys. J. 73, 3182-3191.

Meyer, D. B. & May, H. C., Jr. The topographical distribution of rods and cones in the adult chicken retina. Experimental Eye Research 17, 347-55 (1973).

Meyers, S. M. A twin study on age-related macular degeneration. Transactions of the American Ophthalmological Society 92, 775-843 (1994).

Michel and Westhof, J. Mol. Biol., 216:585-610, 1990.

Midena, E., Degli Angeli, C., Blarzino, M. C., Valenti, M. & Segato, T. Macular function impairment in eyes with early age-related macular degeneration. Invest. Ophthalmol. Vis. Sci. 38, 469-77 (1997).

Miksicek et al., Cell, 46:203, 1986.

Miljanich, G. P., Nemes, P. P., White, D. L., and Dratz, E. A. (1981). The asymmetric transmembrane distribution of phosphatidylethanolamine, phosphatidylserine, and fatty acids of the bovine retinal rod outer segment disk membrane. J. Membr. Biol. 60, 249-255.

Moisseiev J, Alhalel A, Masuri R, Treister G. The impact of the macular photocoagulation study results on the treatment of exudative age-related macular degeneration. Arch Ophthalmol 1995; 113: 185-189.

Molday, L. L., Rabin, A. R. & Molday, R. S. ABCR expression in foveal cone photoreceptors and its role in Stargardt macular dystrophy. Nature Genetics 25, 257-258 (2000).

Mordacq and Linzer, Genes and Dev., 3:760, 1989.

Moreau et al., Nucl. Acids Res., 9:6047, 1981.

Muesing et al., Cell, 48:691, 1987.

Nakamura et al., In: Enzyme Immunoassays: Heterogeneous and Homogeneous Systems, Chapter 27, 1987.

Nakatani, K., Tamura, T., and Yau, K. W. (1991). Light adaptation in retinal rods of the rabbit and two other nonprimate mammals. J. Gen. Physiol. 97, 413-435.

Ng et al., Nuc. Acids Res., 17:601, 1989.

Nicotra, C. M. A. et al. Retinoid dynamics in chicken eye during pre- and postnatal development. Molecular and Cellular Biochemistry 132, 45-55 (1994).

Nomoto S, Tatematsu Y, Takahashi T, Osada H. Cloning and characterization of the alternative promoter regions of the human LIMK2 gene responsible for alternative transcripts with tissue-specific expression. Gene Aug. 20, 1999; 236(2):259-71.

Ohara et al., “One-sided polymerase chain reaction: the amplification of cDNA,”

Ondek et al., EMBO J., 6:1017, 1987.

Ornitz et al., Mol. Cell. Biol., 7:3466, 1987.

O'Shannessy et al., J. Immun. Meth., 99, 153-161, 1987.

Owens & Haley, J. Biol. Chem., 259:14843-14848, 1987.

Palmiter et al., Nature, 300:611, 1982.

Palukaitis et al., Virology, 99:145-151, 1979.

Papermaster, D. S., Schneider, B. G., Zorn, M. A., and Kraehenbuhl, J. P. (1978). Immunocytochemical localization of a large intrinsic membrane protein tot he incisures and margins of frog rod outer segment disks. J. Cell Biol. 78, 415-425.

Papermaster, D. S., Schneider, B. G., Zorn, M. A. & Kraehenbuhl, J. P. Immunocytochemical localization of a large intrinsic membrane protein to the incisures and margins of frog rod outer segment disks. J Cell Biol 78, 415-25 (1978).

Parish, C. A., Hashimoto, M., Nakanishi, K., Dillon, J., and Sparrow, J. (1998). Isolation and one-step preparation of A2E and iso-A2E, fluorophores from human retinal pigment epithelium. Proc. Natl. Acad. Sci. USA 95, 14609-14613.

PCT Application No. PCT/US87/00880

PCT Application No. PCT/US89/01025

PCT Application No. WO 88/10315

PCT Application WO 89/06700

PCT Application WO 90/07641

Pech et al., Mol. Cell. Biol., 9:396, 1989.

Peltoketo, H., Isomaa, V., Mäentausta, O., and Vihko, R. (1988) FEBS Lett. 239, 73-77

Pepperberg, D. R., Birch, D. G., and Hood, D. C. (1997). Photoresponses of human rods in vivo derived from paired-flash electroretinograms. Vis. Neurosci. 14, 73-82.

Perez-Stable and Constantini, Mol. Cell. Biol., 10:1116, 1990.

Perriman. et al., Gene, 113:157-163, 1992.

Perrotta and Been, Biochemistry 31:16, 1992.

Persson, B., Krook, M., and Jörnvall, H. (1991) Eur. J. Biochem. 200, 5 37-543

Picard and Schaffner, Nature, 307:83, 1984.

Pinkert et al., Genes and Dev., 1:268, 1987.

Poincelot, R. P., Millar, P. G., Kimbel, R. L., Jr., and Abrahamson, E. W. (1969). Lipid to protein chromophore transfer in the photolysis of visual pigments. Nature 221, 256-257.

Ponta et al., Proc. Nat'l Acad. Sci. USA., 82:1020, 1985.

Porton et al., Mol. Cell. Biol., 10: 1076, 1990.

Potter & Haley, Meth. in Enzymol., 91, 613-633, 1983.

Proc Natl Acad Sci USA. 86(15):5673-5677, 1989.

Prody, G. A. et al., Science, 231, 1577-15 80, 1986.

Queen and Baltimore, Cell, 35:741, 1983.

Quinn et al., Mol. Cell. Biol., 9:4713, 1989.

Rando, R. R. & Chang, A. Studies on the catalyzed interconversion of vitamin A derivatives. J. Am. Chem. Soc. 105, 2879-2882 (1983).

Rando, R. R. Molecular mechanisms in visual pigment regeneration. Photochemistry & Photobiology 56, 1145-56 (1992).

Rawlings, M., and Cronan, J. E., Jr. (1992) J. Biol. Chem. 267, 5751-5754 [Abstract]

Redmond, T. M. et al. Rpe65 is necessary for production of 11-cis-vitamin A in the retinal visual cycle. Nature Genetics 20, 344-51 (1998).

Redondo et al., Science, 247:1225, 1990.

Reinboth, J. J., Gautschi, K., Munz, K., Eldred, G. E. & Reme, C. E. Lipofuscin in the retina: quantitative assay for an unprecedented autofluorescent compound (pyridinium bis-retinoid, A2-E) of ocular age pigment. Experimental Eye Research 65, 639-43 (1997).

Reinhold-Hurek and Shub, Nature, 357:173-176, 1992.

Reisman and Rotter, Mol. Cell. Biol., 9:3571, 1989.

Remington's Pharmaceutical Sciences, 18th Ed. Mack Printing Company, 1990.

Resendez Jr. et al., Mol. Cell. Biol., 8:4579, 1988.

Ripe et al., Mol. Cell. Biol., 9:2224, 1989.

Rittling et al., Nucl. Acids Res., 17:1619, 1989.

Rodriguez, K. A. & Tsin, A. T. Retinyl esters in the vertebrate neuroretina. American Journal of Physiology 256, R255-8 (1989).

Rosen et al., Cell, 41:813, 1988.

Rozet, J. M., Gerber, S., Souied, E., Perrault, I., Chatelin, S., Ghazi, I., Leowski, C., Dufier, J. L., Munnich, A., and Kaplan, J. (1998). Spectrum of abcr gene mutations in autosomal recessive macular dystrophies. Eur. J. Hum. Genet. 6, 291-295.

Saari, J. C., Garwin, G. G., Van Hoosen, J. P., and Palczewski, K. (1998). Reduction of all-trans-retinal limits regeneration of visual pigment in mice. Vision Res. 38, 1325-1333.

Saari, J. C. Retinoids in Photosensitive Systems in The Retinoids: Biology, Chemistry, and Medicine (eds. Sporn, M. B., Roberts, A. B. & Goodmann, D. S.) 351-385 (Raven Press, New York, 1994).

Saari, J. C., Bredberg, D. L., Garwin, G. G., Buczylko, J., Wheeler, T., Palczewski, K. (1993). Analytical Biochemistry 213:128-132.

Saari, J. C., Garwin, G. G., Haeselleer, F., Jang, G. F., Palczewski, K. (2000). Methods in Enzymology 316B, 359-371.

Sakai, N., Decatur, J., Nakanishi, K. & Eldred, G. E. Ocular Age Pigment “A2-E”: An Unprecedented Pyridinium Bisretinoid. J. Am. Chem. Soc. 118, 1559-1560 (1996).

Sambrook et al., In: Molecular Cloning: A Laboratory Manual, Vol. 1, Cold Spring Harbor Laboratory Press, Cold Spring Harbor, N.Y., Ch. 7,7.19-17.29, 1989.

Sarver, et al., Science, 247:1222-1225, 1990.

Sasso et al., J. Immunol., 142:2778-2783, 1989.

Satake et al., “Biological Activities of Oligonucleotides Spanning the F9 Point Mutation Within the Enhancer Region of Polyoma Virus DNA,” J. Virology, 62:970, 1988.

Scanlon et al., Proc. Nat'l Acad. Sci. USA, 88:10591-10595, 1991.

Schaffner et al., J. Mol. Biol., 201:81, 1988.

Scheit, Nucleotide Analogs John Wiley, New York, 1980

Schutt, F., Davies, S., Kopitz, J., Holz, F. G. & Boulton, M. E. Photodamage to human RPE cells by A2-E, a retinoid component of lipofuscin. Invest. Ophthalmol. Vis. Sci. 41(2000).

Searle et al., Mol. Cell. Biol., 5:1480, 1985.

Sharp and Marciniak, Cell, 59:229, 1989.

Shaul and Ben-Levy, EMBO J., 6:1913, 1987.

Sherman et al., Mol. Cell. Biol., 9:50, 1989.

Shorki et al., J. Immunol., 146:936-940, 1991.

Silvermann et al., J. Clin. Invest., 96:417-426, 1995.

Simon, Andràs et al. “The Retinal Pigment Epithelial-specific 11-cis Retinol Dehydrogenase Belongs to the Family of Short Chain Alcohol Dehydrogenases” JBC Online 270:3 1107-1112 (1995).

Simonelli, F. et al. New ABCR mutations and clinical phenotype in Italian patients with Stargardt disease. Invest. Ophthalmol. Vis. Sci. 41, 892-897 (2000).

Sioud et al., J. Mol. Biol., 223:831-835, 1992.

Sleigh and Lockett, J. EMBO, 4:3831, 1985.

Smit, J. J., Schinkel, A. H., Oude Elferink, R. P., Groen, A. K., Wagenaar, E., van Deemter, L., Mol. C. A., Ottenhoff, R., van der Lugt, N. M., and van Roon, M. A. (1993). Homozygous disruption of the murine mdr2 P-glycoprotein gene leads to a complete absence of phospholipid from bile and to liver disease. Cell 75, 451-462.

Souied, E. H. et al. ABCR gene analysis in familial exudative age-related macular degeneration. Invest. Ophthalmol. Vis. Sci. 41, 244-7 (2000).

Spalholz et al., Cell, 42:183, 1985.

Spandau and Lee, J. Virology, 62:427, 1988.

Spandidos and Wilkie, EMBO J., 2: 1193, 1983.

Sparrow, J. R., Parish, C. A., Hashimoto, M. & Nakanishi, K. A2E, a lipofuscin fluorophore, in human retinal pigmented epithelial cells in culture. Invest. Ophthalmol. Vis. Sci. 40, 2988-2995 (1999).

Stargardt, K. Uber familiare, progressive degeneration in der maculagegend des auges. Graefes Arch Ophthalmol 71, 534-550 (1909).

Steinberg, R. H. Interactions between the retinal pigment epithelium and the neural retina. Doc Ophthalmol 60, 327-46 (1985).

Steinmetz, R. L., Garner, A., Maguire, J. I., and Bird, A. C. (1991). Histopathology of incipient fundus flavimaculatus. Ophthalmology 98, 953-956.

Steinmetz, R. L., Haimovici, R., Jubb, C., Fitzke, F. W. & Bird, A. C. Symptomatic abnormalities of dark adaptation in patients with age-related Bruch's membrane change. British Journal of Ophthalmology 77, 549-54 (1993).

Stephens and Hentschel, Biochem. J., 248:1, 1987.

Stinson, A. M., Wiegand, R. D. & Anderson, R. E. (1991) Exp. Eye Res. 52, 213-8.

Stone, E. M. et al. Allelic Variation in Abcr Associated With Stargardt-Disease But Not Age-Related Macular Degeneration. Nature Genetics 20, 328-329 (1998).

Stuart et al., Nature, 317:828, 1985.

Su, J., Lin, M., Napoli, L. L. (1999). Complementary Deoxyribonucleic Acid Cloning and Enzymatic Characterization of a Novel 17{beta}/3{alpha}-Hydroxysteroid/Retinoid Short Chain Dehydrogenase/Reductase. Endocrinology 140: 5275-5284

Sullivan and Peterlin, Mol. Cell. Biol., 7:3315, 1987.

Sun, H., and Nathans, J. (1997). Stargardt's ABCR is localized to the disc membrane of retinal rod outer segments Nat. Genet. 17, 15-16.

Sun, H., Molday, R. S. & Nathans, J. Retinal Stimulates ATP hydrolysis by purified and reconstituted ABCR, the photoreceptor-specific ATP-binding cassette transporter responsible for Stargardt disease. Journal of Biological Chemistry 274, 8269-81 (1999).

Sundelin, S., Wihlmark, U., Nilsson, S. E. G. & Brunk, U. T. Lipofuscin Accumulation in Cultured Retinal Pigment Epithelial Cells Reduces Their Phagocytic Capacity. Current Eye Research 17, 851-857 (1998).

Surya, A., Foster, K. W., and Knox, B. E. (1995). Transducin activation by the bovine opsin apoprotein. J. Biol. Chem. 270, 5024-5031.

Suzuki, T., Fujita, Y., Noda, Y., and Miyata, S. (1986). A simple procedure for the extraction of the native chromophore of visual pigments: the formaldehyde method. Vision Res. 26, 425-429.

Swartzendruber and Lehman, J. Cell. Physiology, 85:179, 1975.

Symons. Ann. Rev. Biochem., 61:641-671, 1992.

Takebe et al., Mol. Cell. Biol., 8:466, 1988.

Tamura, T., Nakatani, K., and Yau, K. W. (1989). Light adaptation in cat retinal rods. Science 245, 755-758.

Tavenier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Bhem., 264:15160, 1989.

Thiesen et al., J. Virology, 62:614, 1988.

Thompson et al., Nature Medicine. 1:277-278, 1995.

Travis, G. (1998). Mechanisms of cell death in the inherited retinal degenerations Am. J. Hum. Genet. 62, 503-508.

Treatment of Age-Related Macular Degeneration with Photodynamic Therapy (TAP) Study Group. Verteporfin (VisudyneJ) therapy of subfoveal choroidal neovascularization in age-related macular degeneration. One year results of two randomized clinical trials: TAP report No 1. Arch Ophthalmol 1999; 117: 1329-1345.

Treatment of Age-Related Macular Degeneration With Photodynamic Therapy (TAP) Study Group. Photodynamic therapy of subfoveal choroidal neovascularization in age-related macular degeneration with verteporfin: two-year results of 2 randomized clinical trials: TAP report No 2. Arch Ophthalmol (in press).

Trehan, A., Canada, F. J. & Rando, R. R. Inhibitors of retinyl ester formation also prevent the biosynthesis of 11-cis-retinol. Biochemistry 29, 309-12 (1990).

Treisman, Cell, 42:889, 1985.

Tronche et al., Mol. Biol. Med., 7:173, 1990.

Tronche et al., Mol. Cell. Biol., 9:4759, 1989.

Trudel and Constantini, Genes and Dev., 6:954, 1987.

Tsang, S. H., Burns, M. E., Calvert, P. D., Gouras, P., Baylor, D. A., Goff, S. P. & Arshavsky, V. Y. (1998) Science 282, 117-21.

Tsang, S. H., Burns, M. E., Calvert, P. D., Gouras, P., Baylor, D. A., Goff, S. P., and Arshavsky, V. Y. (1998). Role for the target enzyme in deactivation of photoreceptor G protein in vivo. Science 282, 117-121.

Tsumaki N, Kimura T, Tanaka K, Kimura J H, et al. Modular arrangement of cartilage-and neural tissue-specific cis- elements in the mouse alpha2(XI) collagen promoter. J Biol Chem Sep. 4, 1998; 273(36):22861-4.

Tyndall et al., Nuc. Acids. Res., 9:6231, 1981.

U.S. Pat. No. 3,817,837

U.S. Pat. No. 3,817,837

U.S. Pat. No. 3,850,752

U.S. Pat. No. 3,850,752

U.S. Pat. No. 3,939,350

U.S. Pat. No. 3,939,350

U.S. Pat. No. 3,996,345

U.S. Pat. No. 3,996,345

U.S. Pat. No. 4,275,149

U.S. Pat. No. 4,275,149

U.S. Pat. No. 4,277,437

U.S. Pat. No. 4,277,437

U.S. Pat. No. 4,366,241

U.S. Pat. No. 4,366,241

U.S. Pat. No. 4,472,509

U.S. Pat. No. 4,472,509

U.S. Pat. No. 4,683,195

U.S. Pat. No. 4,883,750

U.S. Pat. No. 4,938,948

U.S. Pat. No. 4,938,948

U.S. Pat. No. 4,938,948

U.S. Pat. No. 4,946,773

U.S. Pat. No. 5,021,236

U.S. Pat. No. 5,196,066

U.S. Pat. No. 5,279,721

U.S. Pat. No. 5,840,873

U.S. Pat. No. 5,843,640

U.S. Pat. No. 5,843,650

U.S. Pat. No. 5,843,651

U.S. Pat. No. 5,843,663

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,849,481

U.S. Pat. No. 5,849,483

U.S. Pat. No. 5,849,487

U.S. Pat. No. 5,849,497

U.S. Pat. No. 5,849,546

U.S. Pat. No. 5,849,547

U.S. Pat. No. 5,851,770

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,861,244

U.S. Pat. No. 5,863,732

U.S. Pat. No. 5,863,753

U.S. Pat. No. 5,866,331

U.S. Pat. No. 5,866,337

U.S. Pat. No. 5,866,366

U.S. Pat. No. 5,882,864

U.S. Pat. No. 5,905,024

U.S. Pat. No. 5,910,407

U.S. Pat. No. 5,912,124

U.S. Pat. No. 5,912,145

U.S. Pat. No. 5,912,148

U.S. Pat. No. 5,916,776

U.S. Pat. No. 5,916,779

U.S. Pat. No. 5,919,630

U.S. Pat. No. 5,922,574

U.S. Pat. No. 5,925,517

U.S. Pat. No. 5,925,525

U.S. Pat. No. 5,928,862

U.S. Pat. No. 5,928,869

U.S. Pat. No. 5,928,870

U.S. Pat. No. 5,928,905

U.S. Pat. No. 5,928,906

U.S. Pat. No. 5,929,227

U.S. Pat. No. 5,932,413

U.S. Pat. No. 5,932,451

U.S. Pat. No. 5,935,791

U.S. Pat. No. 5,935,825

U.S. Pat. No. 5,939,291

U.S. Pat. No. 5,942,391

U.S. Pat. No. 4,683,202

U.S. Pat. No. 4,800,159

U.S. Pat. No. 5,849,486

U.S. Pat. No. 5,851,772

U.S. Pat. No. 5,900,481

U.S. Pat. No. 5,919,626

Vannice and Levinson, J. Virology, 62:1305, 1988.

Vasseur et al., Proc. Nat'l Acad. Sci. USA., 77:1068, 1980.

Walker et al., “Strand displacement amplification—an isothermal, in vitro DNA amplification technique,” Nucleic Acids Res. 20(7):1591-1696, 1992

Wang and Calame, Cell, 47:241, 1986.

Weber et al., Cell, 36:983, 1984.

Weinberger et al. Mol. Cell. Biol., 8:988, 1984.

Weng, J. et al. Insights into the function of Rim protein in photoreceptors and etiology of Stargardt's disease from the phenotype in abcr knockout mice. Cell 98, 13-23 (1999).

West, R. W. & Dowling, J. E. Anatomical evidence for cone and rod-like receptors in the gray squirrel, ground squirrel, and prairie dog retinas. J Comp Neurol 159, 439-60 (1975).

Wing, G. L., Blanchard, G. C., and Weiter, J. J. (1978). The topography and age relationship of lipofuscin concentration in the retinal pigment epithelium. Invest. Ophthalmol. Vis. Sci. 17, 601-607.

Winoto and Baltimore, Cell, 59:649, 1989.

Wood, R., and Lee, T. (1983). High performance liquid chromatography of fatty acids: quantitative analysis of saturated monoenoic, polyenoic, and geometrical isomers. J. Chromatogr. 254, 237-246.

Wu H K, Squire J A, Song Q, Weksberg R. Promoter-dependent tissue-specific expressive nature of imprinting gene, insulin-like growth factor II, in human tissues. Biochem Biophys Res Commun Apr. 7, 1997; 233(1):221-6.

Wu, G., and Hubbell, W. L. (1993). Phospholipid asymmetry and transmembrane diffusion in photoreceptor disc membranes. Biochemistry 32, 879-888.

Yamamoto, H. et al. Mutations in the gene encoding 11-cis retinol dehydrogenase cause delayed dark adaptation and fundus albipunctatus. Nature Genetics 22, 188-91 (1999).

Young, R. W. & Bok, D. Participation of the retinal pigment epithelium in the rod outer segment renewal process. Journal of Cell Biology 42, 392-403 (1969).

Yuan and Altman, Science, 263:1269-1273, 1994.

Yuan et al., Proc. Nat'l Acad. Sci. USA, 89:8006-8010, 1992.

Yutzey et al. “An Internal Regulatory Element Controls Troponin I Gene Expression,” Mol. Cell. Biol., 9:1397, 1989.

Yutzey et al. Mol. Cell. Biol., 9:1397, 1989.

Zhao-Emonet J C, Boyer O, Cohen J L, Klatzmann D. Deletional and mutational analyses of the human CD4 gene promoter: characterization of a minimal tissue-specific promoter. Biochem Biophys Acta Nov. 8, 1998; 1442(2-3):109-19.

Zhou, Z., White, K. A., Polissi, A., Georgopoulos, C., and Raetz, C. R. (1998). Function of Escherichia coli MsbA, an essential ABC family transporter, in lipid A and phospholipid biosynthesis. J. Biol. Chem. 273, 12466-12475.

What is claimed is:

1. A method of screening for a therapeutic agent for the treatment of macular or retinal degeneration comprising:
(a) contacting a short chain dehydrogenase with a candidate substance; and
(b) determining whether the candidate substance reduces the activity of the dehydrogenase.

2. A method of screening for a therapeutic agent for the treatment of macular or retinal degeneration comprising:
(a) obtaining a dehydrogenase capable of oxidizing 11 cis-retinol to 11-cis-retinaldehyde;
(b) contacting dehydrogenase with a candidate substance; and
(c) determining whether the candidate substance reduces the activity of the dehydrogenase.

3. The method of claim 2, wherein the dehydrogenase is a short chain dehydrogenase.

4. The method of claim 3, wherein the dehydrogenase is a 11-cis-retinol dehydrogenase.

5. The method of claim 4, wherein the dehydrogenase is a 11-cis retinol dehydrogenase type-5.

6. The method of claim 4, wherein the 11cis-retinol dehydrogenase is a polypeptide comprising at least 30 contiguous amino acids of SEQ ID NO: 4.

7. The method of claim 4, wherein the 11-cis-retinol dehydrogenase is encoded by a nucleic acid segment comprising at least 30 contiguous bases of SEQ ID NO: 3.

8. The method of claim 2, wherein the dehydrogenase is provided by a recombinant host cell.

9. The method of claim 2, wherein the contacting occurs in vitro.

10. The method of claim 2, wherein the activity of the dehydrogenase is enzymatic activity.

11. The method of claim 2, wherein the candidate substance is a small molecule.

12. The method of claim 2, wherein the candidate substance is a peptide, polypeptide, or nucleic acid molecule.

13. The method of claim 12, wherein the polypeptide comprises an antibody, ribozyme or antisense molecule.

14. The method of claim 13, wherein the ribozyme or antisense molecule comprises a portion of the coding sequence of the dehydrogenase.

15. The method of claim 2, further comprising
(c) contacting a first animal lacking a functional abcr gene with the candidate substance; and
(d) determining the amount of a component of lipofuscin in a retinal pigment epithelium cell.

16. The method of claim 15, wherein the component of lipofuscin is A2-E.

17. The method of claim 15, further comprising:
(e) comparing the amount of a component of lipofuscin from the first animal with an amount of a component of lipofuscin in a retinal pigment epithelium cell from a second animal lacking a functional abcr gene in the absence of the candidate substance.

18. The method of claim 15, wherein the first animal is a mouse.

19. The method of claim 18, wherein the mouse has a knockout mutation in an abcr gene.

21. The method of claim 10, wherein step (b) comprises measuring the rate of dehydrogenated product formation.

22. The method of claim 10, wherein step (b) comprises evaluating the amount of transfer of a tritiated label from NAD⁺ to NADH.

23. A method of screening for a therapeutic agent for the treatment of macular or retinal degeneration comprising:
(a) contacting a first cell expressing 11-cis retinol dehydrogenase with a candidate substance;
(b) determining whether the activity of amount of 11-cis-retinol dehydrogenase is reduced in the first cell compared to a second cell expressing 11-cis-retinol dehydrogenase but not contacted with the candidate substance.

24. The method of claim 23, wherein the amount of 11-cis-retinol dehydrogenase is determined by measuring 11-cis-retinol dehydrogenase protein levels.

25. The method of claim 23, wherein the amount of 11-cis-retinol dehydrogenase is determined by measuring 11-cis-retinol dehydrogenase transcript levels.

26. The method of claim 23, wherein the activity of 11-cis-retinol dehydrogenase is enzymatic activity.

27. The method of claim 23, wherein the cell is a recombinant cell.

28. The method of claim 23, wherein the first and second cell, or a parent thereof, is transfected with recombinant 11-cis-retinol dehydrogenase prior to (a).

29. A method of screening for a therapeutic agent for the treatment of macular or retinal degeneration comprising:
(a) contacting a first rod cell with a candidate substance; and
(b) determining the amount of lipofuscin or a component of lipouscin in the first rod cell;
(c) determining the amount of lipofuscin or a component of lipofuscin in a second rod cell not exposed to the candidate substance;
(d) comparing the amount of lipofuscin or a component of lipofuscin between the first rod cell and the second rod cell.

30. The method of claim 29, further comprising:
(e) contacting a first animal lacking a functional abcr gene with the candidate substance; and
(f) determining the amount of lipofuscin or a component of lipofuscin in a retinal pigment epithelium cell of the first animal.

31. The method of claim 30, further comprising:
(g) comparing the amount of lipofuscin or a component of lipofuscin from the first animal with the amount of lipofuscin or a component of lipofuscin in a retinal pigment epithelium cell from a second animal lacking a functional abcr gene in the absence of the candidate substance.

32. A method of preparing a therapeutic agent for the treatment of macular or retinal degeneration in a subject comprising:
(a) contacting 11cis-retinol dehydrogenase with a candidate substance; and
(b) determining whether the candidate substance reduces the activity of the dehydrogenase; and
(c) formulating the candidate substance in a pharmaceutically acceptable formulation, wherein the candidate substance reduces the activity of the dehydrogenase.

33. The method of claim 32, wherein the candidate substance is formulated for oiphthalmic applications.

34. The method of claim 32, further comprising:
(c) contacting a first animal lacking a functional abcr gene with the candidate substance; and
(d) determining the amount of a component of lipofuscin in a retinal pigment epithelium cell.

35. The method of claim 34, wherein the component is A2-E.

36. The method of claim 32, wherein the 11-cis-retinol dehydrogenase is a polypeptide comprising at least 30 contiguous amino acids of SEQ ID NO: 4.

37. The method of claim 32, wherein the 11-cis-retinol dehydrogenase is encoded by a nucleic acid segment comprising at least 30 contiguous bases of SEQ ID NO: 3.

38. The method of claim 32, wherein the dehydrogenase is provided by a recombinant host cell.

39. A method of treating a subject with macular or retinal degeneration comprising administering to the subject a therapeutically effective amount of an inhibitor of 11-cis-retinal dehydrogenase, wherein 11-cis-retinal dehydrogenase activity is reduced.

40. The method of claim 39, wherein the inhibitor is a small molecule.

41. The method of claim 39, wherein the candidate substance is a peptide, polypeptide, or nucleic acid molecule.

42. The method of claim 41, wherein the polypeptide comprises an antibody, ribozyme or antisense molecule.

43. The method of claim 42, wherein the ribozyme or antisense molecule comprises a portion of the coding sequence of the dehydrogenase.

44. The method of claim 39, wherein the inhibitor is 13-cis-retinoic acid.

45. The method of claim 39, wherein the subject has age-related macular degeneration.

46. The method of claim 39, wherein the subject has Stargardt's disease, cone-rod dystrophy, retinitis pigmentosa, or fundus flavimaculatus.

47. The method of claim 39, wherein the inhibitor is administered directly to an eye afflicted with macular or retinal degeneration.

48. The method of claim 47, wherein the inhibitor is perfused into an eye afflicted with macular or retinal degeneration.

49. The method of claim 39, wherein the inhibitor is administered to the subject at least twice.

50. The method of claim 39, further comprising performing surgery on the subject.

51. The method of claim 39, further comprising performing laser photocoagulation therapy on the subject.

52. The method of claim 39, further comprising performing photodynamic therapy on the subject.

53. The method of claim 39, further comprising administering an anti-angiogenic factor to the subject.

54. A method of treating a subject with macular or retinal degeneration comprising administering to the subject a therapeutically effective amount of an 11-cis-retinal dehydrogenase.


SEQUENCE LISTING (24 sequences)

SEQ ID NO: 1 - DNA, 1128 bp, Homo sapiens
  CDS (77)..(1033)
taagcttcgggcgctgtagtacctgccagctttcgccacaggaggctgccacctgtaggtcacttgggctccagctatgtggctgcctcttctgctgggtgccttactctgggcagtgctgtggttgctcagggaccggcagagcctgcccgccagcaatgcctttgtcttcatcaccggctgtgactcaggctttgggcgccttctggcactgcagctggaccagagaggcttccgagtcctggccagctgcctgaccccctccggggccgaggacctgcagcgggtggcctcctcccgcctccacaccaccctgttggatatcactgatccccagagcgtccagcaggcagccaagtgggtggagatgcacgttaaggaagcagggctttttggtctggtgaataatgctggtgtggctggtatcatcggacccacaccatggctgacccgggacgatttccagcgggtgctgaatgtgaacacaatgggtcccatcggggtcacccttgccctgctgcctctgctgcagcaagcccggggccgggtgatcaacatcaccagcgtcctgggtcgcctggcagccaatggtgggggctactgtgtctccaaatttggcctggaggccttctctgacagcctgaggcgggatgtagctcattttgggatacgagtctccatcgtggagcctggcttcttccgaacccctgtgaccaacctggagagtctggagaaaaccctgcaggcctgctgggcacggctgcctcctgccacacaggcccactatgggggggccttcctcaccaagtacctgaaaatgcaacagcgcatcatgaacctgatctgtgacccggacctaaccaaggtgagccgatgcctggagcatgccctgactgctcgacacccccgaacccgctacagcccaggttgggatgccaagctgctctggctgcctgcctcctacctgccagccagcctggtggatgctgtgctcacctgggtccttcccaagcctgcccaagcagtctactgaatccagccttccagcaagagattgtttttcaaggacaaggactttgatttatttctgcccccaccctggtactgcctggtgcctgccacaaaata

SEQ ID NO: 2 - protein, 318 aa, Homo sapiens
MWLPLLLGALLWAVLWLLRDRQSLPASNAFVFITGCDSGFGRLLALQLDQRGFRVLASCLTPSGAEDLQRVASSRLHTTLLDITDPQSVQQAAKWVEMHVKEAGLFGLVNNAGVAGIIGPTPWLTRDDFQRVLNVNTMGPIGVTLALLPLLQQARGRVINITSVLGRLAANGGGYCVSKFGLEAFSDSLRRDVAHFGIRVSIVEPGFFRTPVTNLESLEKTLQACWARLPPATQAHYGGAFLTKYLKMQQRIMNLICDPDLTKVSRCLEHALTARHPRTRYSPGWDAKLLWLPASYLPASLVDAVLTWVLPKPAQAVY

SEQ ID NO: 3 - DNA, 1396 bp, Bos taurus
cctgaccgagaagggggcagagcagctgaggaaccagacatcagacaggcggactggctcttcccccgtctcgtcgactccttggtctgtagtctgtccgtgcagacggtgatcctggacgtcaccaagactgagaatgtcactgcaaccacgtctgccactaggacctgcagtggttctgactcttacagtgacgttggaccaattgggtcaaggagcatgttggggacagaggtccctggggtctggttggttaacccagttcctcgtacaacccctgtctccagggaccccagaccagaataatgctggcatttgcacacccatggcacccaacgagtggctgaccacttattacgaccgtaaacgtgtgggtaccgtgggttgctcaccgactggtaacaggactttgtgaaggttctggatgtgaacctgttgggggtgattgacttgtcctgaaacacttccaagacctacacttggacaacccccactaactggtgacgctgagtctgctgtccctggtgcggaaggcggggccgtgtggtcgcactgcgactcagacgacagggaccacgccttccgccccggcacaccagcacgtctccagtgtcttgggccgggtgtctctagtcctgcaggctactgcatgcagaggtcacagaacccggcccacagagatcaggacgtccgatgacgttatccaagtacggtgtggaggccttctcagactctctcagaagggagctcataggttcatgccacacctccggaagagtctgagagagtcttccctcgagtcctacttcggagtgaaggtggctatgattgagcctggttactttgttacaggatgaagcctcacttccaccgatactaactcggaccaatgaaacaatgcaatatgacccaagatgagggttttattggatacctccaggcattgtggagttatactgggttctactcccaaaataacctatggaggtccgtaacacctatcgggccagcccagagctgaaagaactctatggagaaaacttccctgcttagcccggtcgggtctcgactttcttgagatacctcttttgaagggacgagacttcttgaagacattgagtttactgaaaccacggtggactcagaatctctgaagaacttctgtaactcaaatgactttggtgccacctgagtcttagagtccttggtgaccgactgcatggagcacgccctgactgcctgccaccccccaggaaccactggctgacgtacctcgtgcgggactgacggacggtggggggcacccgatattcagctggctgggacgccaagttcttctggctgcccacgtgggctataagtcgaccgaccctgcggttcaagaagaccgacgggt

SEQ ID NO: 4 - protein, 213 aa, Bos taurus
LTEKGAEQLRNQTSDRLQTVILDVTKTENVTATTNWVKEHVGDRGPWGLVNNAGICTPMAPNEWLTKQDFVVLDVNLLGVIDVTLSLLSLVRKAGPCGRRLQCLGPGVSSPAGYCISKYGVEAFSPGYFVTNMTQDEGFIGYLQALWNRASPELKELYGENFPADFLKTLSLLKPRWTQNLSLVTDCMEHALTACHPRTRYSAGWDAKFFWLP

SEQ ID NO: 5 - DNA, 1800 bp, Rattus norvegicus
  CDS (295)..(1248)
aaggcaacagaaaggcacaggttcatttaagtagacagtcactctttgaatggtcattctggttccggtttctcatccttgtgaagacgcaattcaccggtttttcgagctcggttaggcgtccggagagactcggtcgctggagtcgtgggagcccgcaggagcctcgggcccggggtgagtcacggctgggagcaggagaaggctggagtccctgaccctacaccatctcgtgagcaaatgcccgtctcattttgactcagagtctgtccctctgcttgtcttctacaagccatgtggctctacctgctggcactggtgggcctgtggaacctcctgcgcttcttgagggagaggaaggtggtgagccatctccaagacaagtatgtcttcatcacgggctgtgactcgggctttgggaacctgctggccagacagctggacaggagaggcatgagggtgctggctgcatgtctgactgagaagggagccgagcagctgaggagcaagacatcggacaggctggagacagtgatccttgatgtcaccaagacagagagtattgtggcagccactcagtgggtgaaggagcgagttgggaacagaggactctggggcctggtcaacaatgctggcatctctgtccccgtgggtcccaatgagtggatgagaaaaaaggactttgcaagtgtgctggatgtgaacctgttgggcgtcatagaggtgactctgaacatgctgcccttagtaaggaaggcgaggggccgcgtggtcaacatcgccagcaccatgggccgaatgtctctcgttggtggtggttactgcatctccaagtatggtgtagaggccttctcagactccctcaggagggagctcacctactttggggtgaaggttgccattatagagcctggtgggttcaagaccaatgtcactaatatggagaggctatcagacaacctgaagaagctgtgggaccagaccactgaggaggtcaaggagatctacggcgagaagtttcaggactcctatatgaaagcaatggagtcactggtgaacacgtgctcaggggacctgtctctggtgaccgactgcatggagcacgccctgacttcctgtcaccctcgcacccgctactcacctggttgggatgccaagttcttctacctccccatgagctaccttcccacctttctttcggatgccgtaatccactggggctctgtaaagcctgcccgagccctgtgatcgacatgtgtgcagacttggggaagtaaggcgggtggagggaagataacaatgtggggtccattgttcaccatactcattaaaataattctgcttccgtactactcaaaaccagtaaagttcagaggaaagagccaaaggattctgccaaggagtaagtaatacataggggctgtcaacatcctgtgaagttaggcagcttctccaggcttcattgtggcctatggtgagaatccacagaacggccgaggatgtaaacacctgggatgtgctttggcccccttcctccttaggagatgggtggcctggtacacccatgccgccatgtaagactgatagaatccacttcaccatcacctttctccgtggtgtcaggtgggagggccaccaccagcacctccgtaccctaatctggattcacaggctgtggttaggaggtggttttactagtgggatagaatctgggaaagatagaaaacaacaacaacagttttattttgtcttaaaaccatggctttctttggtttcctttcctttcataattgaaaagcc

SEQ ID NO: 6 - protein, 317 aa, Rattus norvegicus
MWLYLLALVGLWNLLRFLRERKVVSHLQDKYVFITGCDSGFGNLLARQLDRRGMRVLAACLTEKGAEQLRSKTSDRLETVILDVTKTESIVAATQWVKERVGNRGLWGLVNNAGISVPVGPNEWMRKKDFASVLDVNLLGVIEVTLNMLPLVRKARGRVVNIASTMGRMSLVGGGYCISKYGVEAFSDSLRRELTYFGVKVAIIEPGGFKTNVTNMERLSDNLKKLWDQTTEEVKEIYGEKFQDSYMKAMESLVNTCSGDLSLVTDCMEHALTSCHPRTRYSPGWDAKFFYLPMSYLPTFLSDAVIHWGSVKPARAL

SEQ ID NO: 7 - DNA, 1998 bp, Rattus norvegicus
  CDS (229)..(1182)
ggtgctgctgctgcatgtctgactgagaagggagccgagcatctgaggagcaagacatcagaaggctggagtcccttccctgactaggccagagctaaaaggacaaggctcagggctagcctctgcctatctagttcttttctccctgaagcttacaccatcctgtgggagccgatgcccgtctcattttgactcagagtctgtccctctgcttgtcttctacaaaccatgtggctgtacctgctggcactggtgggcctgtggaacctcctgcgcttgttcagggagaggaaggtggtaagccatctccaagacaaatatgtcttcatcacgggctgtgactcaggctttgggaacctgctggccagacagctggacaggagaggcatgagggtgctggctgcatgtctgactgagaagggagccgagcagctgaggagcaagacatcggacaggctggagacagtgatccttgatgtcaccaagacagagagtattgtggcagccactcagtgggtgaaggagcgagttgggaacacaggactctggggcctggtcaacaatgctggcatctccggccacctgggtcccaatgagtggatgaacaaacagaacattgcaagtgtgctggatgtgaacctgttgggcatgatcgaggtgactctgagcacggtgcccttagtgaggaaggcgaggggccgtgtggtcaacgttgccagcatcgcaggccgactgtctttttgtggtggtggttactgcatctccaagtatggtgtagaggccttctcagactccctcaggagagagctctcctattttggggtcaaggtggctattgtagagcctggcttcttcaggactgatgtgaccaatggtgtcacattatcatcaaacttccagatgctgtgggaccagaccagctcagaagtcagggaagtctatggcgagaactacctggcatcttatctgaaaatgctaaatggattggaccagaggtgcaacaaagatctgtctctggtgactgactgcatggagcacgccctgacttcctgtcaccctcgcacccggtactcagctggttgggatgccaagttcttctacctccccatgagctatctgcccacctttcttgtggatgcccttttctactggacttctccaaagcctgagaaagctctctgaagtctttacctatgtacataactgaggagatgttggtacagtgttagggaggggatcctgaggaggaggtcggagtgctgaggagggagggaggtcattcctctggggtttagtccacactcttcattttgctaattttcctgtgctactactcaaggctgactcgactgacccaagaaatagccaaagaattctgcgaagaagttcaaatacaaaagtgctggcagttgcctagcctctgggcaccatgacctaccatggcgatccacacaaatggggagtttgagaccagcttgtaccaaacactgccctgggtccccctgtgcaggaagtgcacatgccctggctagtttgtaccctatgcagcatagaagtgtccctgctctgaagtcacctgtgaccctggtgtcaggtttgagaagaatcactaataattctaacactcatgcttgaaggccaggtcgcagcctgggtgggagagtcggttacttgtggtgagaatatagtagaggatagagacattaattttttttaaatttactttgaagtcatggctcttcttttcttttttggagtcttttcctttttagagcatagcatgaggtgacctcttgttccctgtgatttaaacaatggcattgctggttggtgaggtttggattgcaggggtaaggggaatgtcctgagaagggtgtgcataggccagcaggaatttccttgcccgtctcagcccttctagccactgaggtgggaataactgctgtccattcaaaacttaacaacaacccctaaggcataagagcatgcatgcaggcc

SEQ ID NO: 8 - protein, 317 aa, Rattus norvegicus
MWLYLLALVGLWNLLRLFRERKVVSHLQDKYVFITGCDSGFGNLLARQLDRRGMRVLAACLTEKGAEQLRSKTSDRLETVILDVTKTESIVAATQWVKERVGNTGLWGLVNNAGISGHLGPNEWMNKQNIASVLDVNLLGMIEVTLSTVPLVRKARGRVVNVASIAGRLSFCGGGYCISKYGVEAFSDSLRRELSYFGVKVAIVEPGFFRTDVTNGVTLSSNFQMLWDQTSSEVREVYGENYLASYLKMLNGLDQRCNKDLSLVTDCMEHALTSCHPRTRYSAGWDAKFFYLPMSYLPTFLVDALFYWTSPKPEKAL

SEQ ID NO: 9 - DNA, 1129 bp, Rattus norvegicus
  CDS (67)..(1020)
cgtgtgcagcagatgcccgtctcattttgactcagagtctgtccctctgcttgtcttctacaagccatgtggctctacctgctggcactggtgggcctgtggaacctcctgcgcttgttcagggagaggaaggtggtgagccatctccaagacaagtatgtcttcatcacgggctgtgactcaggctttgggaacctgctggccagacagctggacaggagaggcatgagggtgctggctgcatgtctgactgagaagggagccgagcagctgaggagcaagacatcggacaggctggagacagtgatccttgatgtcaccaagacagagagtattgtggcagccactcagtgggtgaaggagcgagttgggaacagaggactctggggcctggtcaacaatgctggcatctctgtccccatgggtcccaatgagtggatgagaaaaaaggactttgcaagtgtgctggatgtgaacctgttgggcgtcatcgaggtgactctgaacatgctgcccttagtaaggaaggcaaggggccgcgtggtcaacatcgccagcaccatgggccgaatgtctctccttggtggtggttactgcatctccaagtatggtgtagaggccttctcagactccctcaggagggagctcacctactttggggtgaaggtggctattatagagcctggtgggttcaagaccaatgtcactaatatggagaggctatcagacaacctgaagaagctgtgggaccaggccactgaggaggtcaaggagatctacggcgagaagtttcgggactcctatatgaaagcaatggagtcactggtgaacatgtgctcaggggacctgtctctggtaaccgactgcatggagcacgccctgacttcctgtcaccctcgcacccggtactcagctggttgggatgccaagttcttctacctccccatgagctaccttcccacctttctttcggatgccgtaatctactggggctctgtaaagcctgcccgagccctgtgaatctgcacatgtgtgcagacttggggaagtaaggcgggtggagggagataacaatgtggggtccattgttcaccatactcattaaaataattctgcttccgtactcact

SEQ ID NO: 10 - protein, 317 aa, Rattus norvegicus
MWLYLLALVGLWNLLRLFRERKVVSHLQDKYVFITGCDSGFGNLLARQLDRRGMRVLAACLTEKGAEQLRSKTSDRLETVILDVTKTESIVAATQWVKERVGNRGLWGLVNNAGISVPMGPNEWMRKKDFASVLDVNLLGVIEVTLNMLPLVRKARGRVVNIASTMGRMSLLGGGYCISKYGVEAFSDSLRRELTYFGVKVAIIEPGGFKTNVTNMERLSDNLKKLWDQATEEVKEIYGEKFRDSYMKAMESLVNMCSGDLSLVTDCMEHALTSCHPRTRYSAGWDAKFFYLPMSYLPTFLSDAVIYWGSVKPARAL

SEQ ID NO: 11 - DNA, 3282 bp, Mus musculus
  CDS (45)..(998)
ttttgacacagagtatctctctctctgcttgacttctacaagccatgtggctctacctggtggcactggtgggcctgtggacgcttctgcgcttcttcagggtgaggcaggtggtgagccatctccaagacaaatatgtcttcatcacgggctgtgactctggctttgggaccctgctggccagacagctggacaggagaggcatgagggtgctggctgcatgtctgacggagaagggagccgaggagctgaggaacaagacatctgacaggctggagacagtgatcctggatgtcaccaagacagagagtattgtgacagccactcagtgggtgaaggagcatgttgggaacagaggactctggggcctggtcaacaacgctggcatctccaccccctcgggtcccaacgagtggatgaaaaagcaggactttgcacatgtactggatgtgaacctgttgggcatgatcgaggtgactctgagcatgctgcctttagtgaggaaggcgaggggtcgtgtggtcaacgtctccagtgtcatgggtcgagtgtctctctttggtggtggttactgcatctctaagtatggtgtagaggccttctcagactccctcaggagggagctctcctactttggggtgaaggtggctattatagagcctggcttcttcctgaccggtgtgaccagtagtgccagattatgctcaaatacccagatgctgtgggaccagaccagctcagaaatcagggagatctatggcgagaagtacctggcatcctatctgaaaaggctaaacaaattggacaagaggtgcaacaaggacctgtctggggtgactgactgcatggagcatgctctgactgcctgtcaccctcgtacccgatactcagctggctgggatgctaagctcttctacctccccttgagctacctgcccacctttcttgtggatgcccttctctactggacttccctgaagcctgaaaaagccctctgaagtattcacctatgtgcatacctggggagatgtaggtagagtttgagagagagaatatttaggggaaattaggagagttgggggggggattttattactctggggttctagtcaatacacttcatcttgttaattctcctatgacactactcaagactgatgatgaccaaagaaataggcaaagaattctgccaagggattcagttacaaaagagctggctgatgcccagattatgagcatcatggctaccatgaaggtccacacagatgaggaggctgggacaagtttgtgccaagcactctcctgtgtcctcctctgcaggaaatgtacatgccctggctagtttaaacccctatgcaagatggaattatccctgttctgaaatcacctctgtccaaggtactaggtctgagggagttactaataatgccagcacccattccctggtgtcaggttgccaggctctgggtggaaggttcagttcctggtggtgaaacctagcagagtacggagacaccaatttttcatttgtattgaaattatgggtttctttctttttcttttctttcctttttttttgacacttcttatttttaaaggtaatatgaggtgatctctgttgctctgcgttttgaagtatggctttatcctttagtgaagtttggaacacagagctaaggggaatgtcctgagaaggattggcacaggccagcaggctttctttgactgtctcatcccttccagctaccgaggtgggaacaactgctctccattcaaaacttaataataacacataaggcagaagactgtgcatgcaagtccatggacagaatttacctgtaggcctcagcttcacaagtctttttcctgtagaatcactatgtgaccatgtagagagtatccttctcaacactagtctttcatattggacatttttcaaaagaataagggtcttgaacttctcatggcctctgagagctggctctgcctgctgctaggttggtgaggtttaagagtaggctgtgcagtgaaatcatgtgtggaagagacagaatcctgggctaagtacagacttgccctgggaaggggttcccataaatgcctgactcctcttctctcagtagaaatgtgggggaagaggggacgcaggagaaacttgtaaatatacaataatttctggattaatgaaaatagaaattcctctaccctatcagttgcctggttcattcattttacctatgttattttgagttttttaggaccattgagcatatgtgtgtttaacctgtgtgttccatgtgtgggagcacctcagagatgagaagaaagatggagggttgctggttacaagaaacttttagctggctgcagagatggctcagcagttaagggcactgactgctcttccagagctcctgagttcaaatcccagcaaccacgtggtggctcacaaccacctgcaatgggttttgatgccctcttctggtgtctgaagatagcgactgtgtactcacatatataaaataaataaatttttaaaaagaaaagaaaagaaaaatggaaagaagctttcagccatacaatatggctgtcaggaaatgaatttgggttatactgaacagcaggaagcattcttgattttggactcatttcttcgaaccatgtactgatactgcatcttgtttcattttattttgagatgtctcactgcatggcaggtttaccatgaccttgtaagctctagccttactgtcttcatcactacataaatcatgctgtagtacatcaacagcatctctgcatgttgtgccctgtgagaaggtcaggaggctggacctttctcagtatactctggagcatcgtgatggtttcacaggggaaggggcagcacccaggacagatctacagccccaagtatgggtagagactcaagaacatcatgatgagacagaaccatgtgtaattagcaggaagtactctgtaaggtgatcccaggatgcctgcaacatttcccctttcctcctcacccagttttttgttgttgttcttagaagccagtgtactcagagggacatagaatgactcaggctggcacaagaggatgtttcactcaatcatgcatgagaattgcactgtacccaaccaagacccatccctccaacctgagatcttcaactattagaatacaataaaattctggttattagttccaaaaaaaaaaaaaaaaaaaaaaaaaaaaaaaaaaaaaaaaaaaaaaaaaaaaaaaaaaaaaaaaaaaaaaaa

SEQ ID NO: 12 - protein, 317 aa, Mus musculus
MWLYLVALVGLWTLLRFFRVRQVVSHLQDKYVFITGCDSGFGTLLARQLDRRGMRVLAACLTEKGAEELRNKTSDRLETVILDVTKTESIVTATQWVKEHVGNRGLWGLVNNAGISTPSGPNEWMKKQDFAHVLDVNLLGMIEVTLSMLPLVRKARGRVVNVSSVMGRVSLFGGGYCISKYGVEAFSDSLRRELSYFGVKVAIIEPGFFLTGVTSSARLCSNTQMLWDQTSSEIREIYGEKYLASYLKRLNKLDKRCNKDLSGVTDCMEHALTACHPRTRYSAGWDAKLFYLPLSYLPTFLVDALLYWTSLKPEKAL

SEQ ID NO: 13 - DNA, 1714 bp, Homo sapiens
  CDS (250)..(1185)
tgtctatagtaggtgcggagaagaaatgtttggcagatgaagaaaatgaggtgcagagaggatttaagagattctctcttactgcctgttcagggtcagtccagaattgagggtctccgggggagggcagaggctttgccctgcaggggatctgtgggagggggttaggagggagcagatgagcccaggatgaatggtcagagtcaggtgctgcccggcggaggtcacgagtccagggaggggatcaacatggccgctgcaccccggactgtgttgatctccggctgctcatcaggaattggtctggaacttgcagtgcaactggcccatgaccccaagaagcgctaccaggtcgtggccaccatgagggacctggggaagaaggagacactggaggcagctgctggggaggctctggggcagaccctcaccgtggcccagctggacgtgtgcagtgatgagtcggtggcccagtgtctcagctgtatccagggagaagtggacgtgctggtgaataatgctggaatgggcctggtggggcccctggaggggctcagccttgctgccatgcagaatgtctttgacaccaactttttcggagctgtccgtctcgtcaaagctgtgcttccaggcatgaagaggaggcggcagggccacatcgtggtgatcagcagtgtcatgggcctgcagggtgtcatcttcaacgatgtctatgcagcttccaagttcgccctggagggattcttcgaaagcctcgctatccagctgctgcagttcaacatcttcatctccctggtggagccaggccccgtggtcaccgagtttgaggggaagcttctggcgcaggtttctatggctgagttcccaggcactgaccctgagaccctgcactacttccgggacctctatctcccagcctccaggaagctgttttgctccgtgggacagaacccacaggacgtggttcaggccattgtcaacgtcatcagctcgactcgaccacccctgcgccgacagaccaacatccgctactcgccgctgaccacgctcaaaaccgtggattcctctggcagcctgtatgtgcgaacgacccaccgcctcctcttccgctgtccacgcctcctcaaccttggccttcaatgtctgtcctgcggctgcctcccaacgcgggtgcggccaagatgagcagaacagagcttcacgatccccatccctgaacaaccagacctcttcattccacatctaattcaaaggatgaacagactcttcatttattcattctgcaaactccccctcccctcctctgtgcctagacatggctagaatcccagaaggscctttattttcaggcatagactcctctgtggtcacagctggagcacagagagggaccctgggaacttggcctgggaagcccagagcaggaagccacagctgtctctgggaaagcaagggaggcttcctggaggaagcggcattgttatgagccttgaaggaagagacagactctgctacattcaacctcgtgacttcatgatcctgggcctgagaacacaggaatgattcatagcccaccccccacctccatgcatacaccagagctctgtggaccaaggggtcattcctggtcacacactccatcagcaaaatgtatttgagcacacaccagcaatgtatgtatattttttccattaaatatatatttataaccaca

SEQ ID NO: 14 - protein, 311 aa, Homo sapiens
MAAAPRTVLISGCSSGIGLELAVQLAHDPKKRYQVVATMRDLGKKETLEAAAGEALGQTLTVAQLDVCSDESVAQCLSCIQGEVDVLVNNAGMGLVGPLEGLSLAAMQNVFDTNFFGAVRLVKAVLPGMKRRRQGHIVVISSVMGLQGVIFNDVYAASKFALEGFFESLAIQLLQFNIFISLVEPGPVVTEFEGKLLAQVSMAEFPGTDPETLHYFRDLYLPASRKLFCSVGQNPQDVVQAIVNVISSTRPPLRRQTNIRYSPLTTLKTVDSSGSLYVRTTHRLLFRCPRLLNLGLQCLSCGCLPTRVRPR

SEQ ID NO: 15 - DNA, 1872 bp, Bos taurus
  CDS (62)..(1000)
  modified base (56): N = A, C, G or T/U
ggatccaaagaattcggcacgagctggccagtggaggtcacgcgtncagggaggaatcaacatggctgatgcaccccggactgtactcatctcaggatgctcctctgggatcggcttggagctggcagtgcagctggctcatgaccccaggcagcgctaccaggtggtggccaccatgagggacctgggaaagaaggggacactggagacagccgctggggaggctctgggtcagaccctcaccgtggcccagctggacgtgtgcagtgatgagtcagtggcccagtgtctcaactgcatccagggaggggaagtggacgtgctggtgaataatgctggagtgggcctggtggggcccttggaagggctcagcctagctgccatgcagaacgtctttgataccaacttttttggggctgtccggctggtcaaagctgtgcttcccagcatgaagaggaggcgacagggccacatcgtggtggtcagcagtgtcatggggctgcagggtgtcgtgttcaacgaagtctacgcggcctccaagtttgccatggaggggttcttcgaaagtctggctgtccagctgctacagttcaacatcttcatctccctggtggagccaggcccggttgtcacagaatttgagggcaagctcctagagcaggtttccacagccgagttcccaggcaccgaccctgacaccctgagctactttcgagatctgtacctcccagcctccagggagctctttcacaacgtgggacagagcccacaggatgtagccaaggtcatcgtcaaggtcatcggctcggccagaccacccttgcgccgacggaccaacacccgctacactccactgatcgcactcaaggccatggacccctccggcagcctgtatgtgcgaacctcccactgcctgctcttccgctggccacgcctcctcaaccttggccttcggtgcctggcctgcagctgcttccgcaccccagtgtggccccgatgaacagagcctcacccaaccctcctcacccctgaacaaccaagcctctttagttcacacccctctctggatgtacgacccttcactcattcattcattcattcattcaacaaactctgcctgacacatttttgtgtctgggcattgctggaaccccagagagcactcagcctccaggtacagacccctctgtggtcaaggctaggagcagagagagtggaccctaggatcctgtcatcctgggctgaggagcccagatcagagagctggagttgtgtccgggaacgcaaggaagtcttcctggaggagggggcgttgtccctggaccttgaaggatgagacaggctgtctcagtcaacctctggacttcattatcttcagtctaggaatttaggaatgatccaaacattcccatccctcctcccctcctacacacacactcaaagctggtttgactaaggggtttccagacattctggtcacatactccatcagaagggcttcccaggtggtgctagtggtaaagaacctgcctgccaatgtaggagatgtaaaagatgagggttcgattcctaggttgggaaaatcgcctggaggaggacatggcaacccactccaatattcttgcctggagaattcccatggacagaggagcccggcgagctacagtccatagggtcacaaagaattggacatgaaggaagcaacttagcacacatgcaatccatcagtaaaatgtgtttgcatacataccagcaatgtatgtatattttttcataaaatatatatttgcagctaatagtaatcactgtagtaataaagctcaatttctcatgtataaataaaaacattaaaaagaaaaaaaaaaaaaaaaa

SEQ ID NO: 16 - protein, 312 aa, Bos taurus
MADAPRTVLISGCSSGIGLELAVQLAHDPRQRYQVVATMRDLGKKGTLETAAGEALGQTLTVAQLDVCSDESVAQCLNCIQGGEVDVLVNNAGVGLVGPLEGLSLAAMQNVFDTNFFGAVRLVKAVLPSMKRRRQGHIVVVSSVMGLQGVVFNEVYAASKFAMEGFFESLAVQLLQFNIFISLVEPGPVVTEFEGKLLEQVSTAEFPGTDPDTLSYFRDLYLPASRELFHNVGQSPQDVAKVIVKVIGSARPPLRRRTNTRYTPLIALKAMDPSGSLYVRTSHCLLFRWPRLLNLGLRCLACSCFRTPVWPR

SEQ ID NO: 17 - DNA, 386 bp, Homo sapiens
tgagttcaatatttttatttctttacaatgatttcagaagagattacaaagagattaatatacttaaagaatcagactcttgcaaacagtgacatcattaaaaagagcttattttcattaacatgtgattaacaggaaggagatgattggtgagttttcttcgtaaccaggttcactgtggataggaagggcctgccttccttcccaccatggagatcctaaaatcacaagctccaacctccatcaatgatgacagggttaccagttacataagcagattcatcagaagccaaatacacgcagagcatggctatttcttctgcagttgcgaatcttcccgtcttttgtctcttcaggaaatcattccgtgcctcttcaggatttcc

SEQ ID NO: 18 - protein, 343 aa, Homo sapiens
GLRPPPPGRFSRLPGKTLSACDRENGARRPLLLGSTSFIPIGRRTYASAAEPVGSKAVLVTGCDSGFGFSLAKHLHSKGFLVFAGCLMKDKGHDGVKELDSLNSDRLRTVQLNVFRSEEVEKVVGDCPFEPEGPEKGMWGLVNNAGISTFGEVEFTSLETYKQVAEVNLWGTVRMTKSFLPLIRRAKGRVVNISSMLGRMANPARSPYCITKFGVEAFSDCLRYEMYPLGVKVSVVEPGNFIAATSLYNPESIQAIAKKMWEELPEVVRKDYGKKYFDEKIAKMETYCSSGSTDTSPVIDAVTHALTATTPYTRYHPMDYYWWLRMQIMTHLPGAISDMIYIR

SEQ ID NO: 19 - DNA, 1401 bp, Homo sapiens
  CDS (55)..(963)
cgtattgctcggcccggggagtttcgccccctgcccggctccgcggcgcggaggatggcgtggaaacggctgggcgcgctggtgatgttccctctacagatgatctatctggtggtgaaagcagccgtcggactggtgctgcccgccaagctgcgggacctgtcgcgggagaacgtcctcatcaccggcggcgggagaggcatcgggcgtcagctcgcccgcgagttcgcggagcgcggcgccagaaagattgttctctggggccggactgagaaatgcctgaaggagacgacggaggagatccggcagatgggcactgagtgccattacttcatctgtgatgtgggcaaccgggaggaggtgtaccagacggccaaggccgtccgggagaaggtgggtgacatcaccatcctggtgaacaatgccgccgtggtccatgggaagagcctaatggacagtgatgatgatgccctcctcaagtcccaacacatcaacaccctgggccagttctggaccaccaaggccttcctgccgcgtatgctggagctgcagaatggccacatcgtgtgcctcaactccgtgctggcactgtctgccatccccggtgccatcgactactgcacatccaaagcgtcagccttcgccttcatggagagcctgaccctggggctgctggactgtccgggagtcagcgccaccacagtgctgcccttccacaccagcaccgagatgttccagggcatgagagtcaggtttcccaacctctttcccccactgaagccggagacggtggcccggaggacagtggaagctgtgcagctcaaccaggccctcctcctcctcccatggacaatgcatgccctcgttatcttgaaaagcatacttccacaggctgcactcgaggagatccacaaattctcaggaacctacacctgcatgaacactttcaaagggcggacatagagacaggatgaagacatgcttgaggagccacggagtttgggggccacagcacctgggcacacacccgagcacctgtccattggcatgcttctgctgggtgagcaggacagctcctgtccccagcgaagaatccggctgcccctgggccagtcccaggacctttgcacaggactgatgggtgtaactgacccccacagggaggcaggaaaacagccagaagccaccttgacacttttgaacatttccagttctgtagagtttattgtcaattgcttctcaagtctaaccagcctcagcagtgtgcatagaccatttccaggagggtctgtccccagatgctctgcctcccgttccaaaacccactcatcctcagcttgcacaaactggttgaacggcaggaatgaaaaataaagagagatggcttttgtgaaaaaaaaa

SEQ ID NO: 20 - protein, 302 aa, Homo sapiens
MAWKRLGALVMFPLQMIYLVVKAAVGLVLPAKLRDLSRENVLITGGGRGIGRQLAREFAERGARKIVLWGRTEKCLKETTEEIRQMGTECHYFICDVGNREEVYQTAKAVREKVGDITILVNNAAVVHGKSLMDSDDDALLKSQHINTLGQFWTTKAFLPRMLELQNGHIVCLNSVLALSAIPGAIDYCTSKASAFAFMESLTLGLLDCPGVSATTVLPFHTSTEMFQGMRVRFPNLFPPLKPETVARRTVEAVQLNQALLLLPWTMHALVILKSILPQAALEEIHKFSGTYTCMNTFKGRT

SEQ ID NO: 21 - DNA, 2518 bp, Homo sapiens
  CDS (18)..(818)
ccagcagtggctgcaccatgcacgtgaacggcaaagtggcgctggtgaccggcgcggctcagggcataggcagagcctttgcagaggcgctgctgcttaagggcgccaaggtagcgctggtggattggaatcttgaagcaggtgtacagtgtaaagctgccctgcatgagcaatttgaacctcagaagactctgttcatccagtgcgatgtggctgaccagcaacaactgagagacacttttagaaaagttgtagaccactttggaagactggacattttggtcaataatgctggagtgaataatgagaaaaactgggaaaaaactctgcaaattaatttggtttctgttatcagtggaacctatcttggtttggattacatgagtaagcaaaatggaggtgaaggcggcatcattatcaatatgtcatctttagcaggactcatgcccgttgcacagcagccggtttattgtgcttcaaagcatggcatagttggattcacacgctcagcagcgttggctgctaatcttatgaacagtggtgtgagactgaatgccatttgtccaggctttgttaacacagccatccttgaatcaattgaaaaagaagaaaacatgggacaatatatagaatataaggatcatatcaaggatatgattaaatactatggaattttggacccaccattgattgccaatggattgataacactcattgaagatgatgctttaaatggtgctattatgaagatcacaacttctaagggaattcattttcaagactatgatacaactccatttcaagcaaaaacccaatgaacagcttatgtgttagccatagctgaaaataagcacaaatagcttatattcagatcctatcttcatttgaatatagcttttaaatgaaatgttacagtttgaagttttccttcatgcacttggtgataaacgttttctaaatttttagttaagtatatggataaaaagttatgaactattaaaaatgtgatgtggaccaaaggctaggttgtaatcttgatagtctaaaaaatgatcaaaacaaatgattttcaaggaatattcaatattctgcctttcagaaagtgtatttatatctgtgcttcataaatattaatgttcttcagaacatcattttaaaggagatacttgaattgttatttaaatcaaaccagatgtaaaacactcacatacaagttcatactttaaaagaggaaagctacttaacaatgacaaatatttcacaataataatttttacttatataccatctttcaactgaacatttcagttcttccaagagcttcttagagtagtatattttgggggcagtcaaggaataaactacagtgtaaacatatcccagatgaaaactgctgtatggaaaaatgacagaaagtaactgattgacactgttgattcacagttcagcctcctatctgggaaagacatttctttcctctgctcactttaagaacttttaccgactccaaaaatctcaggaattaaacttttaacagttacagcaataaagaatagttagtactccaaaaatattatatttaagatgctcaacaagaaaaaaatgcaaatgtaatatttttttcaaattacttctttattgacttgtccaaatttcaaaagtgcctacccttcaataaaacttttttattctgatctccataaattacttagtcttctatgtatagctatcaaggaaataaaaccaattttgccacagccacaactgtaaatgtttttgtacccatgctgaaactcataacaacacagacataaaaatagctgtgaggttttgctttttttgttgtcagctatcttaagaatcattaaatacacctgctttgggtaaaactctttgcaagcagtaattaacactagtaacagtgaaagcacaagatttccaaatcagtcgttttctcaaaaaaatatcgtataagtgactcatcctgtctgctaactccagacctcccagcttgaagccaaatctttccatgtgagattgatatggatttcctagaagtactggaatgttgtcatatcttgccctattttaattctgctatagaaaacaattgccttcacttttaaggagtaatttgaatattaataactctggtctagattttcatataatgtattaaagacaaagtagtgaacatcaatgaacatctgatagagataaactgtaatcaggcataagcttgtttgtatgttctggcagtgactaatcagtaaatgatgtcggtttgcccagtatcacttatcttctgtatttttcctctgtcgtgtaaatagtataaccttttcatttatggacaattttttggactagtagccttcaatatacattctgctttgaattaattttttcaaatcaataaattatgtagacatttaaaatcaaatatcaagtagaattgaaaaatgtgagttacataagttaaaaacttactttaaatcttaccttctataggtagctc

SEQ ID NO: 22 - protein, 266 aa, Homo sapiens
MHVNGKVALVTGAAQGIGRAFAEALLLKGAKVALVDWNLEAGVQCKAALHEQFEPQKTLFIQCDVADQQQLRDTFRKVVDHFGRLDILVNNAGVNNEKNWEKTLQINLVSVISGTYLGLDYMSKQNGGEGGIIINMSSLAGLMPVAQQPVYCASKHGIVGFTRSAALAANLMNSGVRLNAICPGFVNTAILESIEKEENMGQYIEYKDHIKDMIKYYGILDPPLIANGLITLIEDDALNGAIMKITTSKGIHFQDYDTTPFQAKTQ

SEQ ID NO: 23 - DNA, 26 bp, Bos taurus
  modified_base (18)..(21): N = A, C, G or T/U
  modified_base (24): R =  A or G
  modified_base (25): S = G or C
tgcctgaccgagaagggngcngarsa

SEQ ID NO: 24 - DNA, 28 bp, Bos taurus
  modified_base (20): N = A, C, G or T/U
  modified_base (23): R = A or G
tgggcagccagaagaacttngcrtccca